(19) United States
(12) Patent Application Publication
(10) Pub. No.: US 2007/0213338 A1
(43) Pub. Date: Sep. 13, 2007

(54) Triazolo-Pyridazine Compounds and Derivatives Thereof Useful in the Treatment of Neuropathic Pain

(76) Inventors: Alec D. Lebsack, San Diego (CA); Benito Munoz, San Diego, CA (US); Richard Pracitto, La Jolla, CA (US); Shankar Venkatraman, San Diego, CA (US); Bowei Wang, San Diego, CA (US); Janet Gunzner, Berkeley, CA (US)

Correspondence Address: MERCK AND CO., INC, P O BOX 2000, RAHWAY, NJ, 07065-0907, US

(21) Appl. No.: 10/575,942

(22) PCT Filed: Oct. 15, 2004
(86) PCT No.: PCT/US04/34466

§ 371 Date: Dec. 28, 2006

Related U.S. Application Data
(60) Provisional application No. 60/513,046, filed on Oct. 21, 2003.

(51) Int. Cl. A61K 31/50

(52) U.S. Cl. 514/252.05; 514/252.06; 544/234; 544/235; 544/236

(57) ABSTRACT

The present invention is directed to a method of use of triazolo-pyridazine compounds in the treatment of neuropathic pain. The present invention is also directed to the use of triazolo-pyridazine compounds in the treatment of psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases. The present invention is also directed to novel triazolo-pyridazine compounds that selectively bind to α₂δ-1 subunit of Ca channels.

### BACKGROUND OF THE INVENTION

### FIELD OF THE INVENTION

The present invention is directed triazolo-pyridazine compounds and method of their use. In particular, this invention is directed to a method of use of triazolo-pyridazine compounds in the treatment of neuropathic pain.

### RELATED BACKGROUND

A major mechanism in many physiological processes, including neurotransmission in the mammalian nervous system, is the opening and closing of voltage gated calcium channels (“VGCC”), also known as voltage sensitive calcium channels (“VSCC”). Such VGCC are formed by the assembly of subunit classes such as alpha 1 and alpha 2. One subunit in the alpha 2 class is the α₂δ subunit. The activity of the calcium channel can be modulated by the activities of the component subunits. For example, gabapentin is known to bind with high affinity to the α₂δ subunit. Four isoforms of this α₂δ protein are known and gabapentin binds with high affinity to 2 of these (α₂δ-1 and α₂δ-2). The relative importance of these two activities in accounting for the efficacy and adverse effects of gabapentin is not known. Compounds that display high-affinity binding to the α₂δ subunit of voltage gated calcium channels have been shown to be efficacious for the treatment of, for example, neuropathic pain. See, J. Biol. Chem., 271(10):5768-5776(1996) and J. Med. Chem., 41:1838-1845(1998). Nonetheless, if one isoform is more controlling of the channel modulation, while the other is less, then compounds that are selective to the controlling isoform are likely to be more efficacious and display fewer side-effects.

Thus, it is desirable to identify other compounds that display high-affinity binding to the α₂δ subunit of voltage gated calcium channels to provide new medicines in the treatment of neuropathic pain. Further, such compounds can be useful in the treatment of psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases.

International Patent Publication No. WO 01/88101 describes a cell line for the expression of an α₂δ2 calcium channel subunit.

6-Methyl-6H-pyrrolo[3,4-d]pyridazine is described in M M. J. Duflos et al., Tetrahedron Lett., 3453-3454(1973). 1,4,5,7-tetramethyl-6-phenyl-6H-pyrrolo[3,4d]pyridazine, 1,4,5-trimethyl-6,7-diphenyl-6H-pyrrolo[3,4-d]pyridazine, 5,7-dimethyl-1,4,6-triphenyl-6H-pyrrolo[3,4-d]pyridazine, 5-methyl-1,4,6,7-tetraphenyl-6H-pyrrolo[3,4-d]pyridazine, 1,4-bis-(4-methoxy-phenyl)-5,7-dimethyl-6-phenyl-6H-pyrrolo[3,4-d]pyridazine, 1,4-bis-(4-methoxy-phenyl)-5-methyl-6,7-diphenyl-6H-pyrrolo[3,4-d]pyridazine, and 1,4-diethyl-5,7-dimethyl-6-phenyl-6H-pyrrolo[3,4-d]pyridazine are described in R. Rips et al., J. Org. Chem., 24:551-554(1959). 1,4,5,7-Tetramethyl-6H-pyrrolo[3,4-d]pyridazine, N-(1,4,5,7-tetramethyl-pyrrolo[3,4-d]pyridazin-6-yl)-benzamide, 1,4,5,7-tetramethyl-pyrrolo[3,4-d]pyridazin-6-ylamine picrate, and 1,4,5,7-tetramethyl-pyrrolo[3,4-d]pyridazin-6-ylamine are described in W. L. Mosby, J. Chem. Soc., 3997-4003(1957). 5,7-Dimethyl-6-phenyl-6H-pyrrolo[3,4-d]pyridazine is described in R. Rips et al., J. Org. Chem., 24:372-374(1959).

5,7-Dimethyl-2-phenacyl-6H-pyrrolo[3,4-d]pyridazinium bromide (also known as 5,7-dimethyl-2-(2-oxo-2-phenyl-ethyl)-6H-pyrrolo[3,4-d]pyridazin-2-ium bromide) and 2-(2-methoxycarbonylvinyl)-5,7-dimethyl-6H-pyrrolo[3,4-d]pyridazinium tetrafloroborate are described in F. Fuentes-Rodriguez et al., J. Chem. Res. Miniprint, 11:2901-2914(1987). 5,7-Diphenyl-6H-pyrrolo[3,4-d]pyridazine is described in T. Hernandez et al., J. Chem. Soc., Perkins Trans., 1:899-902(1985), and F. F. Rodriguez et al., J. Chem. Res. Miniprint, 11:3001-3001(1987). 5,6,7-Trimethyl-6H-pyrrolo[3,4-d]pyridazine is described in T. Hernandez et al., J. Chem. Soc., Perkin Trans., 1:899-902(1985), F. Fuentes-Rodriguez et al., J. Chem. Res. Miniprint, 11:2901-2914(1987), and R. von Kreher et al., Agnew Chem., 82:958(1970).

1,4-Diphenyl-7,8,9,10-tetrahydro-pyridazino[4,5-a]indolizine (also known as 1,4-diphenyl-5,6,7,8-tetrahydro-2,3,8a-triaza-fluorene) and 5-methyl-1,4-diphenyl-7,8,9,10-tetrahydro-pyridazino[4,5-a]indolizine (also known as 9-methyl-1,4-diphenyl-5,6,7,8-tetrahydro-2,3,8a-triaza-fluorene) are described in T. Uchida et al., J. Heterocycl. Chem., 15:1303-1307(1978). 6-Benzyl-1,4-diphenyl-5-p-tolyl-6H-pyrrolo[3,4-d]pyridazine, 6-benzyl-5-(2-chloro-phenyl)-1,4-diphenyl6H-pyrrolo[3,4-d]pyridazine, 1,4,5,6,7-pentaphenyl-6H-pyrrolo[3,4-d]pyridazine, 6,7, 10,11-tetraphenyl-pyridazino[4′, 5′:3,4]pyrrolo[1,2-a]quinoxaline (also known as 6,7,10,11-tetraphenyl-5,8,9,11a-tetraaza-benzo [a]fluorene), 11-(4-nitro-phenyl)-6,7,10-triphenyl-pyridazino[4′.5′: 3,4]pyrrolo[1,2-a]quinoxaline (also known as 11-(4-nitro-phenyl)6,7,10-triphenyl-5,8,9,11a-tetraaza-benzo[a]fluorene), and 6-benzyl-1,4,5-triphenyl-6H-pyrrolo[3,4-d]pyridazine are described in T. Uchida et al., J. Heterocycl. Chem., 15:241-248(1978).

9,12-Diphenyl-pyridazino[4′,5′:3,4]pyrrolo[2,1-a]isoquinoline, 5-methylsulfanyl-1,4,6,7-tetraphenyl-6H-pyrrolo[3,4-d]pyridazine, and 1,4,6,7-tetraphenyl-6H-pyrrolo[3,4-d]pyridazine-5-carboxylic acid ethyl ester are described in K. T. Potts et al., J. Org. Chem., 42:1639-1644(1977). 7,10-Diphenyl-pyridazino[4′,5′:3,4]pyrrolo[1,2-a]quinoline, and 11,14-diphenyl-pyridazino[4′,5′:3,4]pyrrolo[1,2-f]phenanthridine (also known as 9,12-diphenyl-10,11,13a-triaza-indeno[1,2-l]phenanthrene) are described in K. T. Potts et al., J. Org. Chem., 44:977-979(1979).

1-Oxo-7-oxy-6b,11b-dihydro(pyridazino[4′,5′-c]-pyrrolo)[2.1-c]benzoxazine-1,4 (also known as 11-hydroxy-5-oxa-8,9,11a-triaza-benzo[a]fluoren-6-one) is described in Kumashiro et al., Nippon Kagaku Zasshi., 82: 1072-1074(1961). 10-Methyl-1,4-diphenyl-8,9-dihydro-7H-benzo(ef)pyridazino[4,5-a]cycl[3.3.2]azine, and 11-methyl-1,4-diphenyl-7,8,9,10-tetrahydrocyclohepta(ef)pyridazino[4,5-a]cycl[3.3.2]azine are described in M. Noguchi et al., J. Heterocycl. Chem., 22:1049-1053(1985).

1,4-Dichloro-5,6,7-trimethyl-6H-pyrrolo[3,4-d]pyridazine, 1-chloro-4-ethoxy-5,6,7-trimethyl-6H-pyrrolo[3,4-d]pyridazine, 1-chloro-5,6,7-trimethyl-6H-pyrrolo[3,4-d]pyridazinium chloride, 1-ethoxy-2,5,6,7-tetramethyl-6H-pyrrolo[3,4-d]pyridazinium tetrafluoroborate, 1-ethoxy-5,6,7-trimethyl-2H,6H-pyrrolo[3,4-d]pyridazinium tetrafluoroborate, 1-ethoxy-3-ethyl-5,6,7-trimethyl-6H-pyrrolo[3,4-d]pyridazinium tetrafluoroborate, and 1-ethoxy-5,6,7-trimethyl-6H-pyrrolo[3,4-d]pyridazine are described in S. Inel et al., Tetrahedron, 40:3979-3986(1984).

5-Cyano-1,4-dimethylpyridazino[4,5-a]indolizine (also known as 1,4-dimethyl-2,3,8a-triaza-fluorene-9-carbonitrile), 1,4-dimethyl-6-phenyl-2,3,8a-triaza-fluorene-9-carbonitrile, 6-benzolyl-1,4-dimethyl-2,3,8a-triaza-fluorene-9-carbonitrile, 6-benzyl-1,4-diphenyl-2,3,8a-triaza-fluorene-9-carbonitrile, and 1,4,6-trimethyl-2,3,8a-triaza-fluorene-9-carbonitrile are described in K. Matsumoto et al., J. Heterocycl. Chem., 25:1793-1801(1988). 5-Cyano-1,4-diphenylpyridazino[4,5-a]indolizine (also known as 1,4-diphenyl-2,3,8a-triaza-fluorene-9-carbonitrile) is described in K. Matsumoto et al., J. Heterocycl. Chem., 25:1793-1801(1988), and K. Matsumoto et al., Heterocycles, 20:1525-1529(1983). 6-Methyl-1,4-diphenyl-2,3,8a-triaza-fluorene-9-carbonitrile, 6-benzoyl-1,4-diphenyl-2,3,8a-triaza-fluorene-9-carbonitrile, and 1,4,6-triphenyl-2,3,8a-triaza-fluorene-9-carbonitrile are described in K. Matsumoto et al., J. Heterocycl. Chem, 25:1793-1801(1988), K. Matsumoto et al., Heterocycles, 34:2239-2242(1992), K. Matsumoto et al., Heterocycles, 20:1525-1529(1983), and K. Matsumoto et al., Car, J. Chem., 71:529-533(1993). 5,7-Dimethyl-1,4-diphenyl-2,3,8a-triaza-fluorene-9-carbonitrile, and 9,12-diphenyl-pyridazino[4′,5′:3,4]pyrrolo[2,1-a]isoquinoline-8-carbonitrile are described in K. Matsumoto et al., Heterocycles, 34:2239-2242(1992), and K. Matsumoto et al., Can J. Chem, 71:529-533(1993).

Dimethyl 3,12,13,17-tetramethyl-7²,7³-diazabenzo[g]porphyrin-2,18-dipropionate is described in I. A. Chaudhry et al., Aust. J. Chem, 35:1185-11201(1982). 5,6-Dihydro-2,3-dimethoxypyridazino[4′,5′:3,4]pyrrolo[2,1-a]isochinolin-9-ol, 5,6-dihydro-2,3-dimethoxypyridazino[4′,5′:3,4]pyrrolo[2,1-a]isochinolin-9-ol-hydrochloride, and 3-methyl-6,9-diphenylthiazolo[3′,2′:1,2]pyrrolo[3,4-d]pyridine (also known as 1-methyl-4,7-diphenyl-3-thia-5,6,8a-triaza-cyclopenta[a]indene) are described in W. Losel et al., Chem. Ber., 118:413-427 (1985). 1,4-Diphenylpyridazino[4′,5′:3,4]pyrrolo[2,1-b]benzothiazole (also known as 1,4-diphenyl-5-thia-2,3,9b-triaza-indeno[2,1-a]indene) is described in N. Abe et al., Bull. Chem. Soc. Japan, 55:200-203(1982).

Nevertheless, there is a need to identify 6H-pyrrolo[3,4-d]pyridazine compounds that display high-affinity binding—particularly selective binding—to the α₂δ subunit of voltage gated calcium channels to provide new medicines in the treatment of neuropathic pain, as well as psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases.

### SUMMARY OF THE INVENTION

The present invention is directed to a method of use of triazolo-pyridazine compounds in the treatment of neuropathic pain. The present invention is also directed to the use of triazolo-pyridazine compounds in the treatment of psychiatric and mood disorders such as, for example, schizophrenia, anxiety, depression, bipolar disorders, and panic, as well as in the treatment of pain, Parkinson's disease, cognitive dysfunction, epilepsy, circadian rhythm and sleep disorders—such as shift-work induced sleep disorder and jet-lag, drug addiction, drug abuse, drug withdrawal and other diseases. The present invention is also directed to novel triazolo-pyridazine compounds that selectively bind to α₂δ-1 subunit of Ca channels.

### DETAILED DESCRIPTION OF THE INVENTION

In one aspect the invention is directed to compounds of Formula (I) and Formula II: 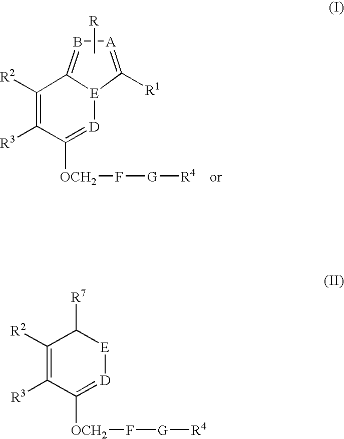
and pharmaceutically acceptable salts thereof, wherein
A and B are each independently selected from the group consisting of CH₂, N and O;
D and E are each independently selected from the group consisting of CH₂, N and O;
F is selected from the group consisting of phenyl and heteroaryl;
G a bond or methylene, wherein the methylene optionally substituted with one or two substituents selected from methyl, ethyl, isopropyl, and carbonyl;
R is selected from the group consisting of
   (a) H,
   (b) CF₃,
   (c) CH₃;
R¹ is selected from the group consisting of
   (a) hydrogen,
   (b) CF₃,
   (c) phenyl,
   (d) —C₁₋₆alkyl,
   (e) —C₃₋₆cycloalkyl,
   (f) —C₂₋₆alkenyl,
   (g) —C₂₋₆alkynyl,
   (h) —O—C₁₋₆alkyl,
   (i) —O—C₂₋₆alkenyl,
   (j) —S—C₁₋₆alkyl, and
   (k) a heterocycloalkyl or heteroaromatic ring of 5 or 6 members, wherein the heterocycloalkyl or heteroaromatic ring comprises 1, 2 or 3 heteroatoms independently selected from the group consisting of N, and O, wherein the heteroaryl is optionally substituted with one or two substituents selected from methyl, methoxy, hydroxyl or halo;
   (1)
R² is selected from the group consisting of
   (a) hydrogen,
   (b) —C₁₋₆alkyl,
   (c) heteroaromatic ring of 5 or 6 members, wherein the heterocycloalkyl or heteroaromatic ring comprises 1, 2 or 3 heteroatoms independently selected from the group consisting of N, and O,
   (d) aryl, and
   (e) —NR⁵R⁶;
R³ is selected from the group consisting of
   (a) hydrogen,
   (b) —C₁₋₆alkyl,
   (c) heteroaromatic ring of 5 or 6 members, wherein the heterocycloalkyl or heteroaromatic ring comprises 1, 2 or 3 heteroatoms independently selected from the group consisting of N, and O,
   (d) aryl, and
   (e) —NR⁵R⁶,
wherein R² and R³ choices (b), (c), (d) and (e) are each optionally substituted with one or two substituents selected from methyl, methoxy, hydroxyl or halo,
or R² and R³ are joined so that together with the atoms to which they are attached there is formed a ring selected from phenyl and cyclohexyl;
R⁴ is —NH(C₁₋₃alkylaryl), optionally substituted with one or two substituents selected from the group consisting of halo, hydroxyl, —C₁₋₆alkyl and —O—C₁₋₆alkyl;
R⁷ is selected from the group consisting of
   (a) hydroxyl,
   (b) C₁₋₃alkoxy,
   (c) N(CH₃)₂,
   (d) Aryl,
   (e) a heteroaromatic ring of 5 or 6 members, wherein the heteroaromatic ring comprises 1, 2 or 3 heteroatoms independently selected from the group consisting of N and O,
      wherein R7 choices (b), (c), (d) and (e) is optionally substituted with one or two substituents selected from methyl, methoxy, hydroxyl and halo.

Within this aspect there is a genus of compounds of Formula (I) 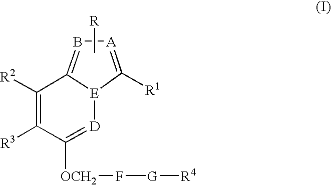
or a pharmaceutically acceptable salt thereof.

Within this genus, there is a sub-genus wherein:
D and E are N.

Within this genus, there is another sub-genus wherein:
G is methylene, wherein the methylene optionally substituted with a substituent selected from methyl, ethyl, isopropyl, and carbonyl.

Within this genus, there is another sub-genus wherein:
R¹ is selected from the group consisting of
   (a) hydrogen,
   (b) CF₃,
   (c) phenyl,
   (d) —C₁₋₃alkyl.

Within this genus, there is another sub-genus wherein:
R² and R³ are joined so that together with the atoms to which they are attached there is formed a ring selected from phenyl and cyclohexyl.

Within this genus, there is another sub-genus wherein:
At least one of R² and R³ is phenyl.

Within this genus, there is another sub-genus wherein:
R⁴ is —NHCH₂CH₂phenyl, optionally substituted with one or two substituents selected from the group consisting of halo, hydroxyl, methyl, ethyl, methoxy and ethoxy;

Within this genus, there is another sub-genus wherein:
A and B are each independently selected from the group consisting of CH₂ and N;
D and E are each independently selected from the group consisting of N and O;
F is selected from the group consisting of phenyl and pyridyl;
G a methylene, wherein the methylene optionally substituted with a substituent selected from methyl, ethyl, isopropyl, and carbonyl;
R is selected from the group consisting of
   (a) H,
   (b) CF₃,
   (c) CH₃;
R¹ is selected from the group consisting of
   (a) hydrogen,
   (b) CF₃,
   (c) phenyl,
   (d) —C₁₋₃alkyl,
R² and R³ are joined so that together with the atoms to which they are attached there is formed a ring selected from phenyl and cyclohexyl;
R⁴ is —NHCH₂CH₂phenyl, optionally substituted with one or two substituents selected from the group consisting of halo, hydroxyl, methyl, ethyl, methoxy and ethoxy;

Within this aspect there is also a genus of compounds of Formula II: 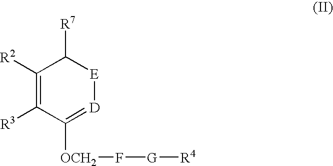
and pharmaceutically acceptable salts thereof.

Within this genus, there is a sub-genus wherein:
D and E are N.

Within this genus, there is another sub-genus wherein:
G is methylene, wherein the methylene optionally substituted with a substituent selected from methyl, ethyl, isopropyl, and carbonyl.
R⁴ is —NHCH₂CH₂phenyl, optionally substituted with one or two substituents selected from the group consisting of halo, hydroxyl, methyl, ethyl, methoxy and ethoxy;

Within this genus, there is another sub-genus wherein:
R⁷ is a pyrrole, pyridine and imidazole optionally substituted with methyl, methoxy, hydroxyl or halo.

Within this genus, there is another sub-genus wherein:
D and E are N;
G is methylene, wherein the methylene optionally substituted with a substituent selected from methyl, ethyl, isopropyl, and carbonyl;
R⁴ is —NHCH₂CH₂phenyl, optionally substituted with one or two substituents selected from the group consisting of halo, hydroxyl, methyl, ethyl, methoxy and ethoxy;
R⁷ is a pyrrole, pyridine and imidazole optionally substituted with methyl, methoxy, hydroxyl or halo.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “aryl” means an aromatic substituent which is a single ring or multiple rings fused together. When formed of multiple rings, at least one of the constituent rings is aromatic. The preferred aryl substituents are phenyl and naphthyl groups.

The term “cycloalkyloxy” unless specifically stated otherwise includes a cycloalkyl group connected by a short C₁₋₂alkyl length to the oxy connecting atom.

The term “C₀₋₆alkyl” includes alkyls containing 6, 5, 4, 3, 2, 1, or no carbon atoms. An alkyl with no carbon atoms is a hydrogen atom substituent when the alkyl is a terminal group and is a direct bond when the alkyl is a bridging group.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

The term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH—if a bridging group and a —NH₂ if a terminal group. Analogous bridging or terminal groups are clear for an O or S heteroatom The term “amine” unless specifically stated otherwise includes primary, secondary and tertiary amines substituted with C₀₋₆alkyl.

The term “carbonyl” unless specifically stated otherwise includes a C₀₋₆alkyl substituent group when the carbonyl is terminal.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the aryl and the alkyl groups are optionally substituted. If only one of the multiple moieties is optionally substituted then it will be specifically recited such as “an alkylaryl, the aryl optionally substituted with halogen or hydroxyl.”

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes the use of all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes the use of all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound used in the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions used of 2H-pyrrolo[3,4-c]pyridazine compounds of the present invention comprise a compound represented by Formula I (or pharmaceutically acceptable salts thereof) as an active ingredient, a pharmaceutically acceptable carrier and optionally other therapeutic ingredients or adjuvants. Such additional therapeutic ingredients include, for example, i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAD”), ix) GABA-A receptor modulators, x) dopamine agonists or antagonists, xi) selective serotonin reuptake inhibitors (“SSRI”) and/or selective serotonin and norepinephrine reuptake inhibitors (“SSNRI”), xii) tricyclic antidepressant drugs, xiv) norepinephrine modulators, xv) L-DOPA, xvi) buspirone, xvii) lithium, xviii) valproate, ixx) neurontin (gabapentin), xx) olanzapine, xxi) nicotinic agonists or antagonists including nicotine, xxii) muscarinic agonists or antagonists, xxiii) heroin substituting drugs such as methadone, levo-alpha-acetylmethadol, buprenorphine and naltrexone, and xxiv) disulfiram and acamprosate. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.01 mg/kg to about 140 mg/kg of body weight per day are useful in the treatment of psychiatric and mood disorders such as, for example, schizophrenia, anxiety, depression, panic, bipolar disorders, and circadian disorders, as well as being useful in the treatment of pain which are responsive to calcium channel modulation, or alternatively about 0.5 mg to about 7 g per patient per day. For example, schizophrenia, anxiety, depression, and panic may be effectively treated by the administration of from about 0.01 mg to 75 mg of the compound per kilogram of body weight per day, or alternatively about 0.5 mg to about 3.5 g per patient per day. Pain may be effectively treated by the administration of from about 0.01 mg to 125 mg of the compound per kilogram of body weight per day, or alternatively about 0.5 mg to about 5.5 g per patient per day. Further, it is understood that the calcium channel modulating compounds of this invention can be administered at prophylactically effective dosage levels to prevent the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds used represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions used in the present invention can be presented as discrete units suitable for oral administration such as capsules, cachets or tablets each containing a predetermined amount of the active ingredient Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions used in this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions used in the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions used in this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions used in this invention have been found to exhibit biological activity as calcium channel ligands. Accordingly, another aspect of the invention is the treatment in mammals of, for example, schizophrenia, anxiety, depression, panic, bipolar disorders, circadian rhythm and sleep disorders, pain, Parkinson's disease, cognitive dysfunction, epilepsy, drug addiction, drug abuse and drug withdrawal—maladies that are amenable to amelioration through modulation of the calcium channel—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound used in this invention can be utilized in combination with other therapeutic compounds. In particular, the combinations of the calcium channel modulating compound used in this invention can be advantageously used in combination with i) opiate agonists or antagonists, ii) mGluR5 antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSRI”) and/or selective serotonin and norepinephrine reuptake inhibitors (“SSNRI”), xii) tricyclic antidepressant drugs, xiii) norepinephrine modulators, xiv) L-DOPA, xv) buspirone, xvi) lithium, xvii) valproate, xviii) neurontin (gabapentin), xix) olanzapine, xx) nicotinic agonists or antagonists including nicotine, xxi) muscarinic agonists or antagonists, xxii) heroin substituting drugs such as methadone, levo-alpha-acetylmethadol, buprenorphine and naltrexone, and xxiii) disulfiram and acamprosate.

The abbreviations used herein have the following tabulated meanings. Abbreviations not tabulated below have their meanings as commonly used unless specifically stated otherwise.

| | |
|---|---|
| Ac | Acetyl |
| AIBN | 2,2′-azobis(isobutyronitrile) |
| BINAP | 1,1′-bi-2-naphthol |
| Bn | Benzyl |
| CAMP | cyclic adenosine-3′,5′-monophosphate |
| DAST | (diethylamino)sulfur trifluoride |
| DEAD | diethyl azodicarboxylate |
| DBU | 1,8-diazabicyclo[5.4.0]undec-7-ene |
| DIBAL | diisobutylaluminum hydride |
| DMAP | 4-(dimethylamino)pyridine |
| DMF | N,N-dimethylformamide |
| Dppf | 1,1′-bis(diphenylphosphino)-ferrocene |
| EDCI | 1-(3-dimethylaminopropyl)-3-ethylcarbodiimide |
| | hydrochloride |
| Et₃N | Triethylamine |
| GST | glutathione transferase |
| HMDS | Hexamethyldisilazide |
| LDA | lithium diisopropylamide |
| m-CPBA | metachloroperbenzoic acid |
| MMPP | monoperoxyphthalic acid |
| MPPM | monoperoxyphthalic acid, magnesium salt 6H₂O |
| Ms | methanesulfonyl = mesyl = SO₂Me |
| Ms0 | methanesulfonate = mesylate |
| NBS | N-bromo succinimide |
| NSAID | non-steroidal anti-inflammatory drug |
| o-Tol | ortho-tolyl |
| OXONE ® | 2KHSO₅•KHSO₄•K₂SO₄ |
| PCC | pyridinium chlorochromate |
| Pd₂(dba)₃ | Bis(dibenzylideneacetone)palladium(0) |
| PDC | pyridinium dichromate |
| PDE | Phosphodiesterase |
| Ph | Phenyl |
| Phe | Benzenediyl |
| PMB | Para-methoxybenzyl |
| Pye | Pyridinediyl |
| r.t. | Room temperature |
| Rac. | Racemic |
| SAM | aminosulfonyl or sulfonamide or SO₂NH₂ |
| SEM | 2-(trimethylsilyl)ethoxymethoxy |
| SPA | scintillation proximity assay |
| TBAF | Tetra-n-butylammonium fluoride |
| Th | 2- or 3-thienyl |
| TFA | trifluoroacetic acid |
| TFAA | trifluoroacetic acid anhydride |
| THF | Tetrahydrofuran |
| Thi | Thiophenediyl |
| TLC | thin layer chromatography |
| TMS-CN | trimethylsilyl cyanide |
| TMSI | trimethylsilyl iodide |
| Tz | 1H (or 2H)-tetrazol-5-yl |
| XANTPHOS | 4,5-Bis-diphenylphosphanyl-9,9-dimethyl-9H-xanthene |
| C₃H₅ | Allyl |

Alkyl Group Abbreviations

| | |
|---|---|
| Me = | Methyl |
| Et = | Ethyl |
| n-Pr = | normal propyl |
| i-Pr = | isopropyl |
| n-Bu = | normal butyl |
| i-Bu = | Isobutyl |
| s-Bu = | secondary butyl |
| t-Bu = | tertiary butyl |
| c-Pr = | cyclopropyl |
| c-Bu = | cyclobutyl |
| c-Pen = | cyclopentyl |
| c-Hex = | cyclohexyl |

### Assays Demonstrating Biological Activity

The compounds of this invention were tested by the following assays.

Membrane Preparation:

A710 (HEK293 co-expressing □1b, □2□, □3) cultured in T250 flask were harvested and washed once with buffer A (20 mM HEPES 10 mM EDTA pH=7.4). The pellet was homogenized in buffer A using a Polytron for 20s. After centrifugation for 10 min, the resulting pellet was washed once with the same buffer and twice with buffer B (20 mM HEPES 0.1 mM EDTA pH=7.4). The final pellet was resuspended in the same buffer and aliquoted and stored at −70° C. Protein contents was measured by the Biorad D C method with bovine serum albumin used as standard.

[³H]-GABApentin binding:

After thawing, the membranes were washed one time with buffer C (50 mM TRIS pH=7.1) and resuspended in ice cold assay buffer (20 mM HEPES pH=7.4), to have a final protein concentration of 50 □g of protein/well. For the competitive binding experiments, the membranes were incubated with 7 nM [³H]-GABApentin for 1 h at rt in the absence or the presence of at least 11 concentrations of the compounds to be tested. The non-specific binding was measured in the presence of 100 □M GABApentin. At the end of the incubation, the suspension was filtered onto 96 well Whatmann GF/B filter plate (Packard) and washed 3 times with ice-cold assay buffer. The plate was dried and 50 □L of microscint 20 (Packard) was added in each well. The plate was sealed and was counted using a Packard Topcount. The plate was counted (2 min) in normal cpm count mode and transforms in DPM with a constant quench correction.

The compounds of this invention displayed efficacy in the above model by IC₅₀ values of less than 10 μM. The compounds the following table, however, gave IC₅₀ values of more than 10 μM:

Spinal Nerve Ligation Model (Chung Model):

The spinal nerve ligation model of neuropathic pain was used to assess the effects of test compounds on nerve injury-induced tactile allodynia (S. H. Kim and J. M. Chung, Pain 50:355-363(1992)). Male Sprague Dawley rats (175-200 g) received unilateral tight ligation of the left L5/L6 spinal nerves distal to the dorsal root ganglion using 4-0 silk suture. Behavioral nociceptive testing occurred 7-14 days following spinal nerve ligation by placing the rats in chambers on a wire mesh. Rats were tested for tactile allodynia (decreased hindpaw withdrawal threshold to non-noxious punctate stimulation) by applying a series of calibrated von Frey filaments to the plantar aspect of the left hindpaw ipsilateral to the site of nerve injury. The mean 50% hindpaw withdrawal threshold (g.) was determined using the Dixon “up-own” non-parametric test (Chaplan et al., J. Neurosci. Methods, 53:55-63(1994)). Rats that displayed a pre-drug withdrawal threshold >4 g were not considered allodynic and were excluded from the study. Following determination of pre-drug withdrawal thresholds, rats received either an i.p. or p.o. injection of test compound. The effect of the test compound on tactile allodynia was determined over time by measuring hindpaw withdrawal thresholds 30, 60, 90, 120 min post-injection.

In above model, EXAMPLE 23 produced a 65% effect after i.p. dosing at 30 mg/kg, EXAMPLE 58 produced a 100% effect after i.p. dosing at 30 mg/kg.

α-Arylaminoacids as an Antagonist of Gabapentin

In this assay, compounds are tested to evaluate whether they may reduce pain by mimicking the mechanism of action of gabapentin. In overview, test compounds are administered alone and in combination with phenylglycine. Compounds whose pain reducing ability is diminished by the addition of phenylglycine are regarded as gabapentin mimics.

Materials and Methods

Male Sprague Dawley rats (Harlan, San Diego, Calif.) weighing 200-250 g were used in the experiments at the time of testing. Rats were housed 3 per cage. All rats were maintained on a standard 12 hr light dark cycle, and had free access to food and water. The experimental procedures described in the present study were approved by the Merck Institutional Animal Care and Use Committee and were performed in accordance with The Guide for the Care and Use of Laboratory Animals.

L5/L6 Spinal Nerve Ligation Injury

Rats were anesthetized with isoflurane (4-5% induction, 2-3% maintenance). Using aseptic technique, the left paraspinal muscles were dissected from the spinous processes at the levels of L4-S2, and the left L5 and L6 spinal nerves were isolated. Each spinal nerve was tightly ligated with a 4-0 silk suture distal to the dorsal root ganglion (Kim and Chung, 1992). Following spinal nerve ligation, the wound was sutured and the skin was closed with veterinarian grade cyanoacrylate. The rats were allowed to recover for 7 days.

Assessment of Mechanical Allodynia

Mechanical allodynia was determined by measuring the paw withdrawal in response to probing with a series of calibrated von Frey filaments. 7-14 days following spinal nerve ligation, rats were placed in individual Plexiglas chambers on an elevated wire mesh where they were allowed to acclimate for 1 hr. Following the acclimation period, rats were tested for tactile allodynia by applying a series of von Frey filaments to the plantar aspect of the left hind paw ipsilateral to the site of nerve injury. The strength of the von Frey stimuli ranged from 0.4 to 15 g. The mean 50% withdrawal threshold (g.) was determined using the Dixon “up-down” method (Chaplan et al., 1994; Dixon, 1968). Rats that displayed a pre-drug withdrawal threshold >4 g. were not considered allodynic and were excluded from the study. Following determination of pre-drug withdrawal thresholds, rats received a subcutaneous injection of Gabapentin (GBP, 100 mg/kg) or vehicle. The effects on tactile allodynia were determined over time by measuring hind paw withdrawal thresholds 30, 60, 90, 120 min post-injection. For the experiments examining the effects of Phenylglycine on the antiallodynic action of GBP, Phenylglycine (20 mg/kg) or vehicle was injected i.p. 30 min after GBP or vehicle injection.

Data Analysis and Statistics

All behavioral experimental groups consisted of 5-7 rats. For all experiments the data were represented as mean ±SEM of the response. Statistical analysis of drug effect was performed by comparing post-drug response to pre-drug response using a one-way ANOVA with Dunnett's test and a two way ANOVA with Student-Newman-Keuls Method for post hoc comparisons. Data were converted to % antiallodynia by the formula: % antiallodynia=100×(test value −control value)/(15 g −control value). A computer program was used to calculate the dose required producing a 50% inhibition of the allodynic response at the time of maximal effect.

Reagents

The reagents used in the present experiments were (S) phenylglycine, (D) phenylglycine (Merck Research Laboratories) and gabapentin (Sigma Chemical Co., St. Lous, Mo.). Gabapentin was dissolved in 0.9% saline (pH ˜7), both (S) and (D) phenylglycine were dissolved in saline (pH˜5).

The examples that follow are intended as an illustration of certain preferred embodiments of the invention and no limitation of the invention is implied.

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) and reaction times are given for illustration only. Melting points are uncorrected and ‘d’ indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (□) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. broad; etc. In addition, “Ar” signifies an aromatic signal. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (milllimoles), eq (equivalent(s)).

### Methods of Synthesis

Compounds of the present invention can be prepared according to the following methods. The substituents are the same as in Formula I except where defined otherwise.

### EXAMPLES 1-58

### Example 1

(S)-Phenethyl-{1-[6-(3-trifluoromethyl-[1,2,4]triazolo[3,4-a]phthalazin-6-yloxymethyl)-pyridin-2-yl]-ethyl}-amine

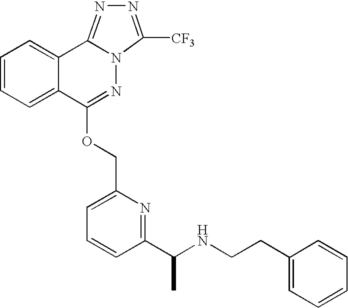

### Step 1: Synthesis of 6-(tert-Butyl-dimethyl-silanyloxymethyl)-pyridine-2-carbaldehyde

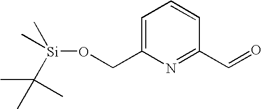

Dess-Martin periodinane (J. Org. Chem. 1983, 48, 4155) (2.0 g, 4.7 mmol) was added to a solution of [6-(tert-butyl-dimethyl-silanyloxymethyl)-pyridin-2-yl]-methanol (J. Org. Chem. 1993, 58, 4389) (1.0 g, 4.3 mmol) and CH₂Cl₂ (20 mL). After 1 h, the reaction was partitioned between saturated aqueous NaHCO₃ (15 mL) and CH₂Cl₂ (20 mL). The layers were separated and the aqueous layer was extracted with CH₂Cl₂ (3×25 mL). The combined organic layers were dried (MgSO₄), filtered, and concentrated. The residue was purified on silica gel (1:99→30:70 ethyl acetate-hexanes) to afford 6-(tert-butyl-dimethyl-silanyloxymethyl)-pyridine-2-carbaldehyde as a colorless oil: ¹H NMR (500 MHz, CDCl₃) δ 10.0 (s, 1H), 7.87 (t, 1H), 7.81 (d, 1H), 7.74 (d, 1H), 4.91 (s, 2H), 0.96 (s, 9H), 0.14 (s, 6H); LRMS (ESI) m/z 252 (252 calcd for C₁₃H₂₂NO₂Si, M+H).

### Step 2: Synthesis of (rac)-1-[6-(tert-Butyl-dimethyl-silanyloxymethyl)-pyridin-2-yl]-ethanol

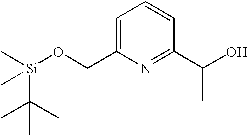

A THF solution of methyl magnesium bromide (3.1 mL, 1.4 M) was added dropwise to 6-(tert-butyl-dimethyl-silanyloxymethyl)-pyridine-2-carbaldehyde (1.0 g, 3.9 mmol) and THF (30 mL) at −78° C. After 2 h, the resulting solution was allowed to warm to 0° C. over 30 min. The reaction was partitioned between saturated aqueous NaHCO₃ (20 mL) and Et₂O (20 mL). The layers were separated and the aqueous layer was extracted with Et₂O (3×20 mL). The combined organic layers were dried (MgSO₄), filtered, and concentrated. The residue was purified on silica gel (1:99→30:70 ethyl acetate-hexanes) to afford (rac)-1-[6(tert-butyl-dimethyl-silanyloxymethyl)-pyridin-2-yl]-ethanol as a colorless oil: ¹H NMR (500 MHz, CDCl₃) δ 7.72 (t, 1H), 7.42 (d, 1H), 7.12 (d, 1H), 4.88-4.84 (m, 3H), 4.45 (br d, 1H), 1.50 (d, 3H), 0.98 (s, 9H), 0.14 (s, 6H); LRMS (ESI) m/z 268 (268 calcd for C₁₄H₂₆NO₂Si, M+H).

### Step 3: Synthesis of 1-[6-(tert-Butyl-dimethyl-silanyloxymethyl)-pyridin-2-yl]-ethanone

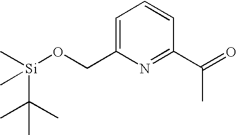

Dess-Martin periodinane (J. Org. Chem. 1983, 48, 4155) (1.9 g, 4.4 mmol) was added to a solution of 1-[6-(tert-butyl-dimethyl-silanyloxymethyl)-pyridin-2-yl]-ethanol (1.0 g, 3.9 mmol) and CH₂Cl₂ (30 mL). After 1 h, the reaction was partitioned between saturated aqueous NaHCO₃ (20 mL) and CH₂Cl₂ (20 mL). The layers were separated and the aqueous layer was extracted with CH₂Cl₂ (3×30 mL). The combined organic layers were dried (MgSO₄), filtered, and concentrated. The residue was purified on silica gel (1:99→1:3 ethyl acetate-hexanes) to afford 1-[6-(tert-butyl-dimethyl-silanyloxymethyl)-pyridin-2-yl]-ethanone as a colorless oil: ¹H NMR (500 MHz, CDCl₃) δ 7.89 (d, 1H), 7.82 (t, 1H ), 7.66 (d, 1H ), 4.87 (s, 2H), 2.68 (s, 3H), 0.97 (s, 9H), 0.14 (s, 6H); LRMS (ESI) m/z 266 (266 calcd for C₁₄H₂₄NO₂Si, M+H).

### Step 4: Synthesis of (R)-1-[6-(tert-Butyl-dimethyl-silanyloxymethyl)-pyridin-2-yl]-ethanol

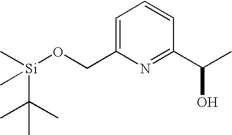

A toluene solution of (S)-tetrahydro-1-methyl-3,3-diphenyl-1H ,3H-pyrrolo[1,2-c][1,3,2]oxazaborole (3 mL, 1 M) was added dropwise to a solution of BH₃.THF (4.1 mL, 1 M) and THF (10 mL) at rt and stirred for 30 min. The solution was cooled to −25° C. whereupon a solution of 1-[6-(tert-butyl-dimethyl-silanyloxymethyl)-pyridin-2-yl]-ethanone (1.0 g, 3.7 mmol) and THF (20 mL) was added by syringe pump over 20 min. After 2 h at −25° C., the solution was allowed to warm to 0° C. over 1 h. The resulting solution was partitioned between saturated aqueous NaHCO₃ (30 mL) and Et₂O (30 mL). The layers were separated and the aqueous layer was extracted with Et₂O (2×40 mL). The combined organic extracts were dried (MgSO₄), filtered, and concentrated. The residue was purified on silica gel (1:99→1:3 ethyl acetate-hexanes) to afford (R)-1-[6-(tert-butyl-dimethyl-silanyloxymethyl)-pyridin-2-yl]-ethanol as a colorless oil: ¹H NMR (500 MHz, CDCl₃) δ 7.72 (t, 1H), 7.42 (d, 1H), 7.12 (d, 1H), 4.88-4.84 (m, 3H), 4.45 (br d, 1H), 1.50 (d, 3H), 0.98 (s, 9H), 0.14 (s, 6H); LRMS (ESI) m/z 268 (268 calcd for C₁₄H₂₆NO₂Si, M+H). Enantiomeric excess of (R)-1-[6-(tert-butyl-dimethyl-silanyloxymethyl)-pyridin-2-yl]-ethanol was determined in Step 9.

### Step 5: Synthesis of (S)-2-(1-Azido-ethyl)-6-(tert-butyl-dimethyl-silanyloxymethyl)-pyridine

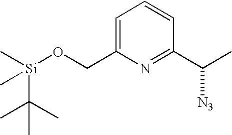

A solution of diethyl azodicarboxylate (1.1 mL, 6.7 mmol) and THF (2 mL) was added dropwise to (R)-1-[6-(tert-butyl-dimethyl-silanyloxymethyl)-pyridin-2-yl]-ethanol (0.9 g, 3.3 mmol) (For an alternative preparation of enantioenriched alcohol see: J. Org. Chem. 1998, 63, 2481), triphenylphosphine (1.8 g, 6.7 mmol), diphenylphosphoryl azide (1.9 g, 6.7 mmol) and THF (30 mL) at rt. After 12 h, the solution was concentrated and the residue was purified on silica gel (1:99→1:2 ethyl acetate-hexanes) to afford (S)-2-(1-azido-ethyl)-6(tert-butyl-dimethyl-silanyloxymethyl)-pyridine as a colorless oil: ¹H NMR (500 MHz, CDCl₃) δ 7.69 (t, 1H ), 7.43 (d, 1H), 7.17 (d, 1H), 4.81 (s, 2H), 4.59 (q, 1H), 1.55 (d, 3H), 0.94 (s, 9H), 0.11 (s, 6H); LRMS (ESI) m/z 293 (293 calcd for C₁₄H₂₅N₄OSi, M+H).

### Step 6: Synthesis of (S)-1-[6-(tert-butyl-dimethyl-silanyloxymethyl)-pyridin-2-yl]-ethylamine

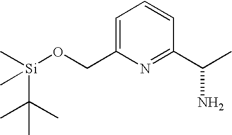

A mixture of (S)-2-(1-azido-ethyl)-6-(tert-butyl-dimethyl-silanyloxymethyl)-pyridine (527 mg, 1.80 mmol), Pd/C (50 mg, 10 wt %), and MeOH (10 mL) was stirred under an atmosphere of hydrogen. After 12 h, the mixture was filtered through Celite, the filter cake was washed with EtOAc (100 mL), and concentrated to afford (S)-1-[6-(tert-butyl-dimethyl-silanyloxymethyl)-pyridin-2-yl]-ethylamine as a clear oil: ¹H NMR (500 MHz, CDCl₃) δ 7.64 (t, 1H), 7.34 (d, 1H), 7.13 (d, 1H), 4.81 (s, 2H), 4.11 (q, 1H), 1.40 (d, 3), 0.95 (s, 9H), 0.11 (s, 6H); LRMS (ESI) m/z 267 (267 calcd for C₁₄H₂₇N₂OSi, M+H).

### Step 7: Synthesis of (S)-{1-[6-(tert-butyl-dimethyl-silanyloxymethyl)-pyridin-2-yl]-ethyl}-phenethyl-amine

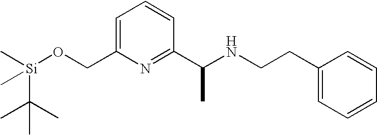

To a solution of (S)-1-[6-(tert-butyl-dimethyl-silanyloxymethyl)-pyridin-2-yl]-ethylamine (513 g, 1.93 mmol), phenylacetaldehyde (231 mg, 1.93 mmol), and dichloroethane (20 mL) was added sodium trisacetoxyborohydride (817 mg, 3.86 mmol). After 36 h, the reaction was partitioned between saturated aqueous NaHCO₃ (20 mL) and CH₂Cl₂ (20 mL). The layers were separated and the aqueous layer was extracted with CH₂Cl₂ (3×30 mL). The combined organic layers were dried (MgSO₄), filtered, and concentrated. The residue was purified on silica gel (1:99→1:4 methanol-CH₂Cl₂) to afford (S)-{1-[6-(tert-butyl-dimethyl-silanyloxymethyl)-pyridin-2-yl]ethyl}-phenethyl-amine as a colorless oil: ¹H NMR (500 MHz, CDCl₃) δ 7.62 (t, 1H), 7.35-7.08 (m, 7H), 4.78 (s, 2H), 3.84 (q, 1H), 2.83-2.63 (m, 4H), 1.33 (d, 3H), 0.96 (s, 9H), 0.12 (s, 6H); LRMS (ESI) m/z 371 (371 calcd for C₂₂H₃₅N₂OSi, M+H).

Step 8: Synthesis of (S)-{1-[6-(tert-butyl-dimethyl-silanyloxymethyl)-pyridin-2-yl]-ethyl}-phenethyl-carbamic acid tert-butyl ester

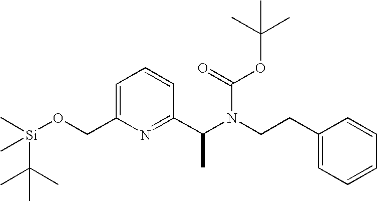

A single portion of di-tert-butyldicarbonate (Boc₂O) was added to (S)-{1-[6-(tert-butyl-dimethyl-silanyloxymethyl)-pyridin-2-yl]-ethyl}-phenethyl-amine (739 mg, 1.99 mmol), triethylamine (0.57 mL, 3.98 mmol), and THF (15 mL). After 12 h, the resulting solution was partitioned between saturated aqueous NaHCO₃ (15 mL) and Et₂O (20 mL). The layers were separated and the aqueous layer was extracted with Et₂O (2×20 mL). The combined organic extracts were dried (MgSO₄), filtered, and concentrated. Diagnostic data for (S)-{1-[6-(tert-butyl-dimethyl-silanyloxymethyl)-pyridin-2-yl]-ethyl}-phenethyl-carbamic acid tert-butyl ester as a colorless oil: ¹H NMR (500 MHz, CDCl₃) δ complex due to the presence of multiple conformations on the NMR time-scale; LRMS (ESI) m/z 371 (371 calcd for C₂₂H₃₅N₂OSi, MH—O₂CC(CH₃)₃).

### Step 9: Synthesis of (S)-[1-(6-Hydroxymethyl-pyridin-2-yl)-ethyl]-phenethyl-carbamic acid tert-butyl ester

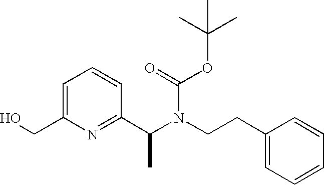

A THF solution of tetrabutylammonium fluoride (2.0 mL, 1M) was added dropwise to (S)-{1-[6-(tert-butyl-dimethyl-silanyloxymethyl)-pyridin-2-yl]ethyl}-phenethyl-carbamic acid tert-butyl ester (0.9 g, 2.0 mmol) and THF (20 mL). After 2 h, the resulting solution was partitioned between saturated aqueous NaHCO₃ (20 mL) and Et₂O (30 mL). The layers were separated and the aqueous layer was extracted with Et₂O (2×30 mL). The combined organic extracts were dried (MgSO₄), filtered, and concentrated. The residue was purified on silica gel (1:99→1:2 ethyl acetate-hexanes) to afford (S)-[1-(6-Hydroxymethyl-pyridin-2-yl)-ethyl]-phenethyl-carbamic acid tert-butyl ester >95% ee (analytical chiral HPLC: Regis Whelk O, 98:1:1 hexane-iso-propanol-triethylamine, 1 mL/min UV detection at 254 nm) as a colorless oil: ¹H NMR (500 MHz, CDCl₃) δ 7.60 (t, 1H), 7.27-7.01 (m, 7H), 4.73 (s, 2H), 3.37-2.30 (m, 4H), 1.6-1.45 (m, 12H); LRMS (ESI) m/z 257 (257 calcd for C₂₁H₂₉N₂O₃, MH—O₂CC(CH₃)₃).

### Step 10: Synthesis of (S)-Phenethyl-{1-[6-(3-trifluoromethyl-[1,2,4]triazolo[3,4-a]phthalazin-6-yloxymethyl)-pyridin-2-yl]-ethyl}-carbamic acid tert-butyl ester

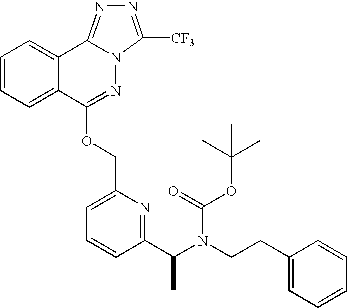

A THF solution of lithium bis(trimethylsilyl)amide (1.20 mL, 1M) was added dropwise to a mixture of (S)-[1-(6-hydroxymethyl-pyridin-2-yl)-ethyl]-phenethyl-carbamic acid tert-butyl ester (380 mg, 1.07 mmol), 6-chloro-3-trifluoromethyl-[1,2,4]triazolo[3,4,-a]phthalazine (319 mg, 1.17 mmol), and DMF (15 mL) at −78° C. After 1 h, the mixture was allowed to warm to 0° C. over 15 min. After 1 h, the resulting solution was partitioned between saturated aqueous NaHCO₃ (40 mL) and Et₂O (60 mL). The layers were separated and the aqueous layer was extracted with Et₂O (2×60 mL). The combined organic extracts were dried (MgSO₄), filtered, and concentrated. The residue was purified on silica gel (5:95→1:1 ethyl acetate-hexanes) to afford (S)-phenethyl-{1-[6-(3-trifluoromethyl-[1,2,4]triazolo[3,4-a]phthalazin-6-yloxymethyl)-pyridin-2-yl]-ethyl}-carbamic acid tert-butyl ester as a yellow oil: ¹H NMR (500 MHz, CDCl₃) δ complex due to the presence of multiple conformations on the NMR time-scale; LRMS (ESI) m/z 493 (493 calcd for C₂₆H₂₄F₃N₆O, MH—O₂CC(CH₃)₃).

### Step 11: Synthesis of (S)-Phenethyl-{1-[6-(3-trifluoromethyl-[1,2,4]triazolo[3,4-a]phthalazin-6-yloxymethyl)-pyridin-2-yl]-ethyl}-amine

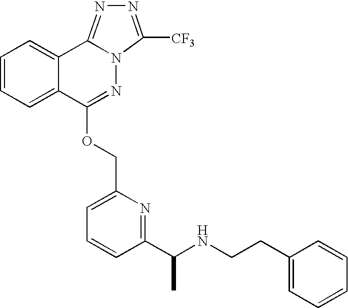

A dioxane solution of HCl (9.0 mL, 4M) was added to (S)-Phenethyl-{1-[6-(3-trifluoromethyl-[1,2,4]triazolo[3,4-a]phthalazin-6-yloxymethyl)-pyridin-2-yl]-ethyl}-carbamic acid tert-butyl ester (0.5 g, 0.9 mmol). After 2 h, the mixture was concentrated and the residue was partitioned between aqueous 0.5 N NaOH (10 mL) and CH₂Cl₂ (20 mL). The layers were separated and the aqueous layer was extracted with CH₂Cl₂ (2×20 mL). The combined organic extracts were dried (MgSO₄), filtered, and concentrated. The residue was purified on silica gel (1:99→1:4 MeOH—CH₂Cl₂) to afford (S)-phenethyl-{1-[6-(3-trifluoromethyl-[1,2,4]triazolo[3,4-a]phthalazin-6-yloxymethyl)-pyridin-2-yl]-ethyl}-amine as a clear oil: ¹H NMR (500 MHz, CDCl₃) δ 8.65 (d, 1H), 8.31 (d, 1H), 7.98 (t, 1H), 7.85 (t, 1H), 7.74 (t, 1H), 7.47 (d, 1H), 7.28 (d, 1H), 7.27-7.14 (m, 5H), 5.61 (s, 2H), 4.02 (q, 1H), 2.90-2.71 (m, 4H), 1.44 (d, 3H); LRMS (ESI) m/z 493 (493 calcd for C₂₆H₂₄F₃N₆O, M+H).

Triated analogs of Example 1 may also be prepared. These include the analogs triated at the 3 position of the alkylphenyl group of Example 1.

### Example 2

### (R)-Phenethyl-{1-[6-(3-trifluoromethyl-[1,2,4]triazolo[3,4-a]phthalazin-6-yloxymethyl)-pyridin-2-yl]-ethyl}-amine

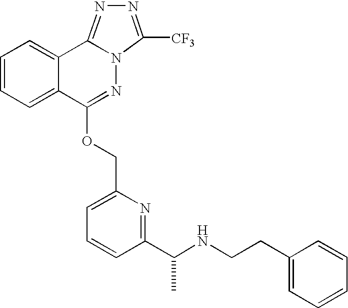

Utilizing the general procedure outlined in Example 1, (S)-tetrahydro-1-methyl-3,3-diphenyl-1H,3H-pyrrolo[1,2-c][1,3,2]oxazaborole in Step 4 was replaced with (R)-tetrahydro-1-methyl-3,3-diphenyl-1H,3H-pyrrolo[1,2-c][1,3,2]oxazaborole to provide (R)-Phenethyl-{1-[6-(3-trifluoromethyl-[1,2,4]triazolo[3,4-a]phthalazin-6-yloxymethyl)-pyridin-2-yl]-ethyl}-amine as a colorless oil: ¹H NMR (500 MHz, CDCl₃) δ 8.65 (d, 1H), 8.31 (d, 1H), 7.98 (t, 1H), 7.85 (t, 1H), 7.74 (t, 1H), 7.47 (d, 1H), 7.28 (d, 1H), 7.27-7.14 (m, 5H), 5.61 (s, 2H), 4.02 (q, 1H), 2.90-2.71 (m, 4H), 1.44 (d, 3H); LRMS (ESI) m/z 493 (493 calcd for C₂₆H₂₄F₃N₆O, M+H).

### Example 3

### (S)-[2-(3-Bromo-phenyl)-ethyl]-{1-[6-(3-trifluoromethyl-3H-[1,2,4]triazolo[3,4-a]phthalazin-6-yloxymethyl)-pyridin-2-yl]-ethyl}-amine

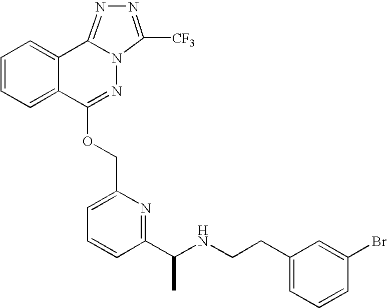

### Step 1: Synthesis of (S)-[2-(3-Bromo-phenyl)-ethyl]-{1-[6-(tert-butyl-dimethyl-silanyloxymethyl)-pyridin-2-yl]-ethyl}-amine

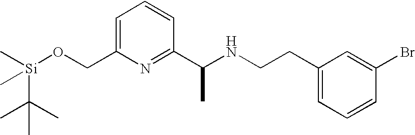

To a solution of (S)-1-[6-(tert-butyl-dimethyl-silanyloxymethyl)-pyridin-2-yl]-ethylamine (see Example 1 for synthesis) (600 mg, 2.26 mmol), 3-bromo-phenylacetaldehyde (PCT Int. Appl. WO 9846605 A1, 1998) (447 mg, 2.26 mmol), and dichloroethane (20 mL) was added NaBH(OAc)₃ (957 mg, 4.52 mmol). After 36 h, the reaction was partitioned between saturated aqueous NaHCO₃ (20 mL) and CH₂Cl₂ (20 mL). The layers were separated and the aqueous layer was extracted with CH₂Cl₂ (3×30 mL). The combined organic layers were dried (MgSO₄), filtered, and concentrated. The residue was purified on silica gel (1:99→1:4 methanol-CH₂Cl₂) to afford (S)-[2-(3-bromo-phenyl)-ethyl]-{1-[6-(tert-butyl-dimethyl-silanyloxymethyl)-pyridin-2-yl]-ethyl}-amine as a colorless oil: ¹H NMR (500 MHz, CDCl₃) δ 7.63 (t, 1H), 7.36-7.30 (m, 3H), 7.14-7.07 (m, 3H), 4.79 (s, 2H), 3.85-3.81 (m, 1H), 2.78-2.63 (m, 4H), 1.33 (d, 3H), 0.96 (s, 9H), 0.12 (s, 6H); LRMS (ESI) m/z 449 (449 calcd for C₂₂H₃₄BrN₂OSi, M+H).

### Step 2: Synthesis of (S)-[2-(3-Bromo-phenyl)-ethyl]-{1-[6-(tert-butyl-dimethyl-silanyloxymethyl)-pyridin-2-yl]-ethyl}-carbamic acid tert-butyl ester

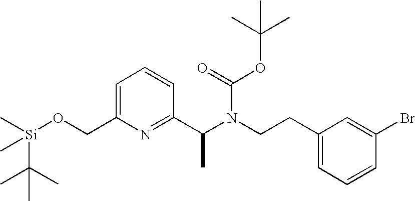

A single portion of di-tert-butyldicarbonate (Boc₂O) (455 mg, 2.08 mmol) was added to (S)-[2-(3-bromo-phenyl)-ethyl]-{1-6-(tert-butyl-dimethyl-silanyloxymethyl)-pyridin-2-yl]-ethyl}-amine (623 mg, 1.39 mmol), triethylamine (0.40 mL, 2.78 mmol), and THF (13 mL). After 12 h, the resulting solution was partitioned between saturated aqueous NaHCO₃ (15 mL) and Et₂O (20 mL). The layers were separated and the aqueous layer was extracted with Et₂O (2×20 mL). The combined organic extracts were dried (MgSO₄), filtered, and concentrated. Diagnostic data for (S)-[2-(3-bromo-phenyl)-ethyl]-{1-[6-(tert-butyl-dimethyl-silanyloxymethyl)-pyridin-2-yl]-ethyl}-carbamic acid tert-butyl ester as a colorless oil: ¹H NMR (500 MHz, CDCl₃) δ complex due to the presence of multiple conformations on the NMR time-scale; LRMS (ESI) m/z 449 (449 calcd for C₂₂H₃₄BrN₂OSi, MH₂—O₂CC(CH₃)₃).

### Step 3: Synthesis of (S)-[2-(3-Bromo-phenyl)-ethyl]-[1-(6-hydroxymethyl-pyridin-2-yl)-ethyl]-carbamic acid tert-butyl ester

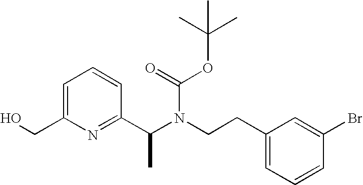

A THF solution of tetrabutylammonium fluoride (1.40 mL, 1M) was added dropwise to (S)-[2-(3-bromo-phenyl)-ethyl]-{1-[6-(tert-butyl-dimethyl-silanyloxymethyl)-pyridin-2-yl]-ethyl}-carbamic acid tert-butyl ester (650 mg, 1.18 mmol) and THF (12 mL). After 2 h, the resulting solution was partitioned between saturated aqueous NaHCO₃ (20 mL) and Et₂O (30 mL). The layers were separated and the aqueous layer was extracted with Et₂O (2×30 mL). The combined organic extracts were dried (MgSO₄), filtered, and concentrated. The residue was purified on silica gel (1:99→1:2 ethyl acetate-hexanes) to afford (S)-[2-(3-bromo-phenyl)-ethyl]-[1-(6-hydroxymethyl-pyridin-2-yl)ethyl]-carbamic acid tert-butyl ester >95% ee (analytical chiral HPLC: Regis Whelk O, 98:1:1 hexane-iso-propanol-triethylamine, 1 mL/min, UV detection at 254 nm) as a colorless oil: ¹H NMR (500 MHz, CDCl₃) δ complex due to the presence of multiple conformations on the NMR time-scale; LRMS (ESI) m/z 435 (435 calcd for C₂₁H₂₈BrN₂O₃, M+H).

### Step 4: Synthesis of (S)-[2-(3-Bromo-phenyl)-ethyl]-{1-[6-(3-trifluoromethyl-3H-[1,2,4]triazolo[3,4-a]phthalazin-6-yloxymethyl)-pyridin-2-yl]-ethyl}-carbamic acid tert-butyl ester

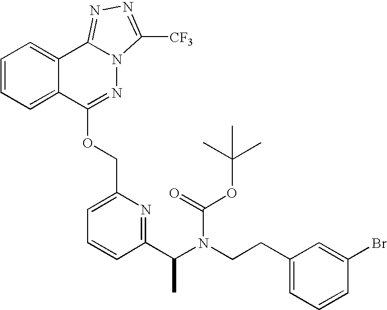

A THF solution of lithium bis(trimethylsilyl)amide (1.20 mL, 1M) was added dropwise to a mixture of (S)-[2-(3-bromo-phenyl)-ethyl]-[1-(6-hydroxymethyl-pyridin-2-yl)-ethyl]-carbamic acid tert-butyl ester (390 mg, 0.89 mmol), 6-chloro-3-trifluoromethyl-[1,2,4]triazolo[3,4,-a]phthalazine (269 mg, 0.99 mmol), and DMF (10 mL) at −78° C. After 1 h, the mixture was allowed to warm to 0° C. over 15 min. After 1 h, the resulting solution was partitioned between saturated aqueous NaHCO₃ (40 mL) and Et₂O (60 mL). The layers were separated and the aqueous layer was extracted with Et₂O (2×60 mL). The combined organic extracts were dried (MgSO₄), filtered, and concentrated. The residue was purified on silica gel (5:95→1:1 ethyl acetate-hexanes) to afford (S)-[2-(3-bromo-phenyl)-ethyl]-{1-[6-(3-trifluoromethyl-3H-[1,2,4]triazolo[3,4-a]phthalazin-6-yloxymethyl)-pyridin-2-yl]-ethyl}-carbamic acid tert-butyl ester as a yellow oil: ¹H NMR (500 MHz, CDCl₃) δ complex due to the presence of multiple conformations on the N time-scale; LRMS (ESI) m/z 571 (571 calcd for C₂₆H₂₃BrF₃N₆O, MH₂—O₂CC(CH₃)₃).

### Step 5: Synthesis of (S)-[2-(3-bromo-phenyl)-ethyl]-{1-[6-(3-trifluoromethyl-3H-[1,2,4]triazolo[3,4-a]phthalazin-6-yloxymethyl)-pyridin-2-yl]-ethyl}-amine

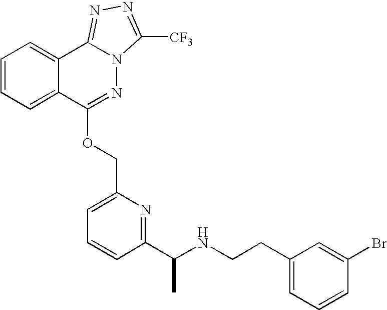

A dioxane solution of HCl (9.0 mL, 4M) was added to (S)-[2-(3-bromo-phenyl)-ethyl]-{1-[6-(3-trifluoromethyl-3H-[1,2,4]triazolo[3,4-α]phthalazin-6-yloxymethyl)-pyridin-2-yl]-ethyl}-carbamic acid tert-butyl ester (0.43 g, 0.64 mmol). After 2 h, the mixture was concentrated and the residue was partitioned between aqueous 0.5 N NaOH (10 mL) and CH₂Cl₂ (20 mL). The layers were separated and the aqueous layer was extracted with CH₂Cl₂ (2×20 mL). The combined organic extracts were dried (MgSO₄), filtered, and concentrated. The residue was purified on silica gel (1:99→1:4 MeOH—CH₂Cl₂) to afford (S)-[2-(3-bromo-phenyl)-ethyl]-{1-[6-(3-trifluoromethyl-3H-[1,2,4]triazolo[3,4-a]phthalazin-6-yloxymethyl)-pyridin-2-yl]-ethyl}-amine as a clear oil: ¹H NMR (500 MHz, CDCl₃) δ 8.67 (d, 1H), 8.32 (d, 1H), 7.99 (t, 1H), 7.87 (t, 1H), 7.74 (t, 1H), 7.46 (d, 1H), 7.31-7.27 (m, 3H), 7.14-7.07 (m, 2H), 5.65 (s, 2H), 3.95-3.91 (m, 1H), 2.83-2.67 (m, 4H), 1.39 (d, 3H); LRMS (ES1) m/z 571 (571 calcd for C₂₆H₂₃BrF₃N₆O, M+H).

### Example 4

### 2-Phenyl-N-[3-({[3-(trifluoromethyl)[1,2,4]triazolo[3,4-a]phthalazin-6-yl]oxy}methyl)-benzyl)ethanamine

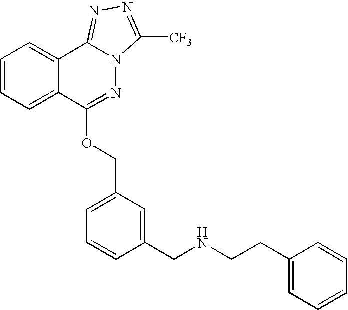

1,4-dichlorophthalazine (1.0 g, 5.0 mmol) and 2,2,2-trifluoroacetohydrazide (0.64 g, 5.0 mmol) were dissolved in dioxane (10 mL) and heated to reflux for 12 h. The reaction mixture was concentrated, dissolved in CH₂Cl₂, washed with a saturated aqueous sodium bicarbonate solution, dried (MgSO₄) to afford the desired 6-chloro-3-trifluoromethyl-[1,2,4]triazolo[3,4-a]phthalazine. The 6-chloro-3-trifluoromethyl-[1,2,4]triazolo[3,4-a]phthalazine (300 mg, 1.1 mmol) and [3-({tert-butyl-dimethyl)silyl]oxy}methyl)phenyl]methanol (Bioorg. Med. Chem. Lett. 12, 2002, 137) (280 mg, 1.1 mmol) were dissolved in DMF (10 mL), cooled to −78° C. and treated with a solution of lithium bis(trimethylsilyl)amide (1.1 mL of 1 M in THF). The reaction was allowed to warm to room temperature over 12 h, concentrated, and the crude product was dissolved in THF (5 mL) and treated with TBAF (1.0 mL of 1 M in THF). After stirring the reaction for 2 h at ambient temperature, the reaction was concentrated and purified by flash column chromatography (SiO₂, 1 to 9% MeOH in CH₂Cl₂) to afford [6-(3-trifluoromethyl-[1,2,4]triazolo[3,4-a]phthalazin-6-yloxymethyl)-pyridin-2-yl]-methanol. The alcohol (224 mg, 0.6 mmol) was dissolved in CH₂Cl₂ (2 mL) and treated with triethylamine (0.17 mL, 1.2 mmol) and methanesulfonyl chloride (0.07 mL, 0.9 mmol). The reaction was stirred at ambient temperature for 1 h, diluted with CH₂Cl₂ (5 mL) and washed with a saturated aqueous sodium bicarbonate solution, dried (MgSO₄), and concentrated to afford [6-({[3-(trifluoromethyl)[1,2,4]triazolo[3,4-a]phthalazin-6-yl]oxy}methyl)pyridin-2-yl]methyl methanesulfonate as a tan solid. The crude mesylate residue was dissolved in methylene chloride (2 mL) and treated with phenethylamine (360 mg, 3.0 mmol) at ambient temperature for 20 h. The reaction was concentrated, and the residue was purified by flash column chromatography (SiO₂, 1%-20% MeOH in CH₂Cl₂) to afford 2-phenyl-N-[3-({[3-(trifluoromethyl)[1,2,4]triazolo[3,4-a]phthalazin-6-yl]oxy}methyl)benzyl]ethanamine as a colorless oil: ¹H NMR (500 MHz, CDCl₃) δ 8.69 (d, 1H), 8.29 (d, 1H), 8.00 (dt, 1H), 7.86 (dt, 1H), 7.50 (s, 1H), 7.48 (d, 1H), 7.40 (m, 1H ), 7.34 (m, 3H), 7.21 (m, 3H), 5.59 (s, 2H), 3.87 (s, 2H), 2.95 (t, 2H), 2.86 (t, 2H); LCMS (ESI) m/z 477 (477 calcd for C₂₆H₂₂F₃N₅O, M+H).

### Example 5

### (2-m-Tolyl-ethyl)-[6-(3-trifluoromethyl-[1,2,4]triazolo[3,4-a]phthalazin-6-yloxymethyl)-pyridin-2-ylmethyl]-amine

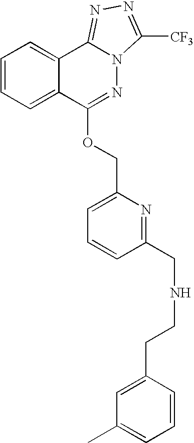

### Step 1: The synthesis of 6-(3-trifluoromethyl-[1,2,4]triazolo[3,4-a]phthalazin-6-yloxymethyl)-pyridine-2-carbaldehyde

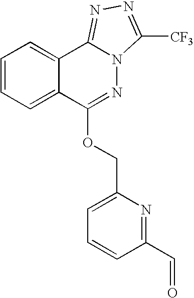

To [6-(3-trifluoromethyl-[1,2,4]triazolo[3,4-a]phthalazin-6-yloxymethyl)-pyridin-2-yl]-methanol (see Example 4) (1.0 g, 2.66 mmol) in CH₂Cl₂ (18 mL) was added Dess-Martin periodinane (1.4 g, 3.2 mmol) (J. Org. Chem. 1993, 58, 4389). After 2 h, the reaction mixture was diluted with CH₂Cl₂ (100 mL) and extracted with 1 M NaOH (2×75 mL). The combine organic layer were dried over MgSO₄, filtered, and concentrated to afford 6-(3-trifluoromethyl-[1,2,4]triazolo[3,4-a]phthalazin-6-yloxymethyl)-pyridine-2-carbaldehyde as a white solid: ¹H NMR (500 MHz, CDCl₃) δ 10.11 (s, 1H), 8.70 (d, 1H), 8.36 (d, 1H ), 8.03 (t, 1H), 8.00 (m, 2H), 7.93 (t, 1H), 7.87 (m, 1H), 5.82 (s, 2H).

### Step 2: Synthesis of (2-m-tolyl-ethyl)-[6-(3-trifluoromethyl-[1,2,4]triazolo[3,4-a]phthalazin-6-yloxymethyl)-pyridin-2-ylmethyl]-amine

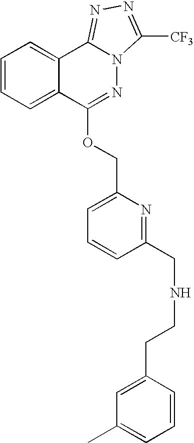

Sodium triacetoxyborohydride (NaHB(OAc)₃) (0.18 g, 0.86 mmol) was added in one portion to 6-(3-trifluoromethyl-[1,2,4]triazolo[3,4-a]phthalazin-6-yloxymethyl)-pyridine-2-carbaldehyde (0.20 g, 0.536 mmol) and 3-methylphenethylamine (0.14 g, 1.07 mmol) in dichloroethane (4 mL) at rt. The mixture was stirred at rt for 20 hours, diluted with CH₂Cl₂ (20 mL) and extracted with 1 M NaOH (1×20 mL). The organic layer was dried over MgSO₄, filtered and concentrated. Purification was performed on silica gel (1:24 MeOH—CH₂Cl₂) to afford (2-m-tolyl-ethyl)-[6-(3-trifluoromethyl-[1,2,4]triazolo[3,4-a]phthalazin-6-yloxymethyl)-pyridin-2-ylmethyl]-amine as a white solid: ¹H NMR (500 MHz, CDCl₃) δ 8.70 (d, 1H), 8.34 (d, 1H), 8.02 (t, 1H), 7.89 (t, 1H), 7.75 (t, 1H), 7.50 (d, 1H), 7.32 (d, 1H), 7.20 (t, 1H), 7.04 (m, 3H), 5.66 (s, 2H), 4.01 (s, 2H), 2.98 (m, 2H), 2.86 (m, 2H), 2.33 (s, 3H).

Triated analogs of Example 5 may also be prepared. These include the analogs triated at the 2 or 4 position of the 3-methylphenyl group of Example 5.

### Example 6

### (2o-Tolyl-ethyl)-[6-(3-trifluoromethyl-[1,2,4]triazolo[3,4-a]phthalazin-6-yloxymethyl)-pyridin-2-ylmethyl]-ammonium

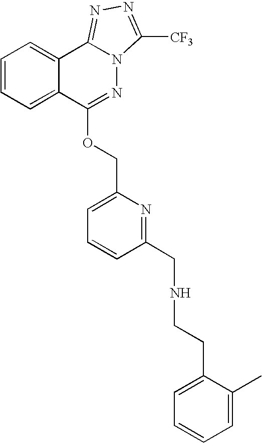

Utilizing the general procedure outlined Example 5, 6-(3-trifluoromethyl-[1,2,4]triazolo[3,4-a]phthalazin-6-yloxymethyl)-pyridine-2-carbaldehyde (0.20 g, 0.54 mmol), 2-methylphenethylamine (0.14 g, 1.07 mmol), sodium trisacetoxyborohydride (0.18 g, 0.86 mmol) and dichloroethane (4 mL) was reacted to give (2o-tolyl-ethyl)-[6-(3-trifluoromethyl-[1,2,4]triazolo[3,4-a]phthalazin-6-yloxymethyl)-pyridin-2-ylmethyl]-ammonium as a white solid: ¹H NMR (500 MHz, CDCl₃) δ 8.67 (d, 1H), 8.33 (d, 1H), 8.02 (t, 1H), 7.87 (t, 1H), 7.75 (t, 1H), 7.50 (d, 1H), 7.32 (d, 1H), 7.16 (t, 1H), 7.13 (m, 3H), 5.66 (s, 2H), 4.01 (s, 2H), 2.94 (m, 2H), 2.89 (m, 2H), 2.32 (s, 3H).

### Example 7

### (2-p-Tolyl-ethyl)-[6-(3-trifluoromethyl-[1,2,4]triazolo[3,4-a]phthalazin-yloxymethyl)-pyridin-2-ylmethyl]-amine

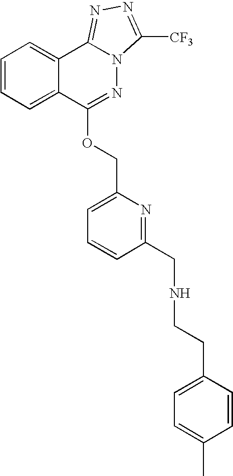

Utilizing the general procedure outlined in Example 5, 6-(3-trifluoromethyl-[1,2,4]triazolo[3,4-a]phthalazin-6-yloxymethyl)-pyridine-2-carbaldehyde (0.20 g, 0.54 mol), 4-methylphenethylamine (0.14 g, 1.07 mmol), sodium trisacetoxyborohydride (0.18 g, 0.86 mmol) and dichloroethane (4 mL) was reacted to give (2-p-tolyl-ethyl)-[6-(3-trifluoromethyl-[1,2,4]triazolo[3,4-a]phthalazin-6-yloxymethyl)-pyridin-2-ylmethyl]-amine as a tan solid: ¹H NMR (500 MHz, CDCl₃) δ 8.70 (d, 1H), 8.34 (d, 1H), 8.03 (t, 1H), 7.88 (t, 1H), 7.74 (t, 1H), 7.48 (d, 1H), 7.32 (d, 1H ), 7.16 (t, 1H), 7.13 (m, 4H), 5.67 (s, 2H), 3.97 (s, 2H), 2.95 (m, 2H), 2.84 (m, 2H), 2.32 (s, 3H).

### Example 8

### [2-(2-Methoxy-phenyl)-ethyl]-[6-(3-trifluoromethyl-[1,2,4]triazolo[3,4-a]phthalazin-6-yloxymethyl)-1,2-dihydro-pyridin-2-ylmethyl]-amine

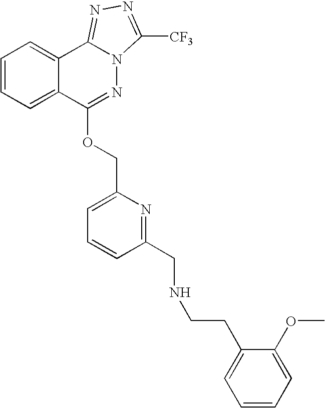

Utilizing the general procedure outlined in Example 5, 6-(3-trifluoromethyl-[1,2,4]triazolo[3,4-a]phthalazin-6-yloxymethyl)-pyridine-2-carbaldehyde (0.1 g, 0.3 mmol), 2-methoxyphenethylamine (0.1 g, 0.6 mmol), sodium trisacetoxyborohydride (0.1 g, 0.5 mmol) and DCE (2 mL) was reacted to give [2-(2-methoxy-phenyl)-ethyl]-[6-(3-trifluoromethyl-[1,2,4]triazolo[3,4-a]phthalazin-6-yloxymethyl)-1,2-dihydro-pyridin-2-ylmethyl]-amine as a tan solid: ¹H NMR (500 MHz, CDCl₃) δ 8.70 (d, 1H), 8.34 (d, 1H), 8.02 (t, 1H), 7.89 (t, 1H), 7.75 (t, 1H), 7.49 (d, 1H), 7.32 (d, 1H), 7.14 (d, 2H), 6.84 (d, 2H), 5.67 (s, 2H), 3.99 (s, 2H), 3.78 (s, 3H), 2.94 (t, 2H), 2.82 (t, 2H).

### Example 9

### [2-(3-Methoxy-phenyl)-ethyl]-[6-(3-trifluoromethyl-[1,2,4]triazolo[3,4-a]phthalazin-6-yloxymethyl)-pyridin-2-ylmethyl]-amine

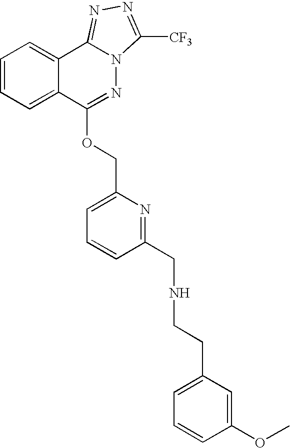

Utilizing the general procedure outlined in Example 5, 6-(3-trifluoromethyl-[1,2,4]triazolo[3,4-a]phthalazin-6-yloxymethyl)-pyridine-2-carbaldehyde (0.1 g, 0.3 mmol), 3-methoxyphenethylamine (0.1 g, 0.6 mmol), sodium trisacetoxyborohydride (0.1 g, 0.5 mmol) and dichloroethane (2 mL) was reacted to give [2-(3-methoxy-phenyl)-ethyl)-[6-(3-trifluoromethyl-[1,2,4]triazolo[3,4-a]phthalazin-6-yloxymethyl)-pyridin-2-ylmethyl]-amine as a white solid: ¹H NMR (500 MHz, CDCl₃) δ 8.70 (d, 1H), 8.33 (d, 1H), 8.01 (t, 1H), 7.88 (t, 1H), 7.75 (t, 1H), 7.49 (d, 1H), 7.32 (d, 1H), 7.23 (t, 1H), 6.82 (d, 1H), 6.77 (m, 2H), 5.66 (s, 2H), 4.00 (s, 2H), 3.79 (s, 3H), 2.99 (t, 2H), 2.86 (t, 2H).

### Example 10

### [2-(4-Methoxy-phenyl)-ethyl]-[6-(3-trifluoromethyl-[1,2,4]triazolo[3,4-a]phthalazin-6-yloxymethyl)-pyridin-2-ylmethyl]-amine

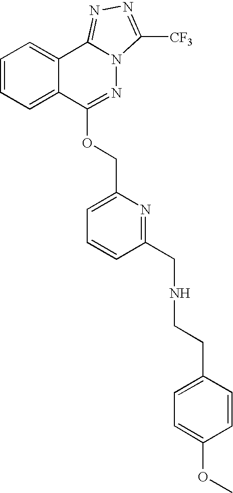

Utilizing the general procedure outlined in Example 5, 6-(3-trifluoromethyl-[1,2,4]triazolo[3,4-a]phthalazin-6-yloxymethyl)-pyridine-2-carbaldehyde (0.1 g, 0.3 mmol), 4-methoxyphenethylamine (0.1 g, 0.6 mmol), sodium trisacetoxyborohydride (0.1 g, 0.5 mmol) and dichloroethane (2 mL) was reacted to afford [2-(4-methoxy-phenyl)-ethyl]-[6-(3-trifluoromethyl-[1,2,4]triazolo[3,4-a]phthalazin-6-yloxymethyl)-pyridin-2-ylmethyl]-amine as a white solid: ¹H NMR (500 MHz, CDCl₃) δ 8.70 (d, 1H), 8.34 (d, 1H ), 8.00 (t, 1H), 7.89 (t, 1H), 7.75 (t, 1H), 7.49 (d, 1H), 7.33 (d, 1H), 7.19 (t, 1H), 7.17 (d, 1H), 6.89 (m, 2H), 5.66 (s, 2H), 4.02 (s, 2H), 3.82 (s, 3H), 2.95 (m, 2H), 2.91 (t, 2H).

### Example 11

### [2-(2-Fluoro-phenyl)-ethyl]-[6-(3-trifluoromethyl-[1,2,4]triazolo[3,4-a]phthalazin-6-yloxymethyl)-pyridin-2-ylmethyl]-amine

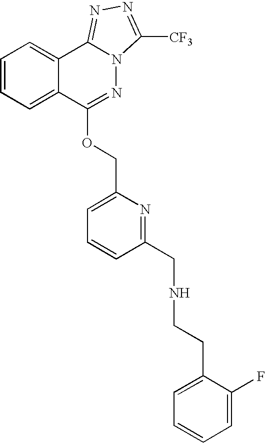

Utilizing the general procedure outlined in Example 5, 6-(3-trifluoromethyl-[1,2,4]triazolo[3,4-a]phthalazin-6-yloxymethyl)-pyridine-2-carbaldehyde (0.1 g, 0.3 mmol), 2-fluorophenethylamine (0.1 g, 0.6 mmol), sodium trisacetoxyborohydride (0.1 g, 0.6 mmol) and dichloroethane (2 mL) was reacted to give [2-(2-fluoro-phenyl)-ethyl]-[6-(3-trifluoromethyl-[1,2,4]triazolo[3,4-a]phthalazin-6-yloxymethyl)-pyridin-2-ylmethyl]-amine as a tan solid: ¹H NMR (500 MHz, CDCl₃) δ 8.71 (d, 1H), 8.35 (d, 1H), 7.91 (t, 1H), 7.76 (t, 1H), 7.51 (d, 1H), 7.33 (d, 1H), 7.19 (t, 1H), 7.23 (m, 2H), 7.07 (m, 2H), 5.69 (s, 2H), 4.02 (s, 2H), 2.98 (t, 2H), 2.93 (t, 2H).

### Example 12

### [2-(3-Fluoro-phenyl)-ethyl]-[6-(3-trifluoromethyl-[1,2,4]triazolo[3,4-a]phthalazin-6-yloxymethyl)-pyridin-2-ylmethyl]-amine

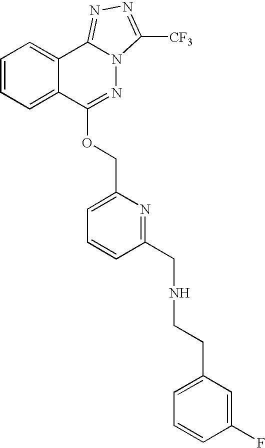

Utilizing the general procedure outlined in Example 5, 6-(3-trifluoromethyl-[1,2,4]triazolo[3,4-a]phthalazin-6-yloxymethyl)-pyridine-2-carbaldehyde (0.1 g, 0.3 mmol), 3-fluorophenethylamine (0.1 g, 0.6 mmol), sodium trisacetoxyborohydride (0.1 g, 0.6 mmol) and dichloroethane (2 mL) was reacted to give [2-(3-fluoro-phenyl)-ethyl]-[6-(3-trifluoromethyl-[1,2,4]triazolo[3,4-a]phthalazin-6-yloxymethyl)-pyridin-2-ylmethyl]-amine as a colorless oil: ¹H NMR (500 MHz, CDCl₃) δ 8.71 (d, 1H), 8.35 (d, 1H), 8.02 (t, 1H), 7.90 (t, 1H), 7.76 (t, 1H), 7.51 (d, 1H), 7.32 (d, 1H), 7.25 (m, 1H), 7.02 (d, 1H), 6.92-(m, 1H), 5.68 (s, 2H), 3.99 (s, 2H), 2.97 (t, 2H), 2.87 (t, 2H).

### Example 13

### [2-(4-Fluoro-phenyl)-ethyl]-[6-(3-trifluoromethyl-[1,2,4]triazolo[3,4-a]phthalazin-6-yloxymethyl)-pyridin-2-ylmethyl]-amine

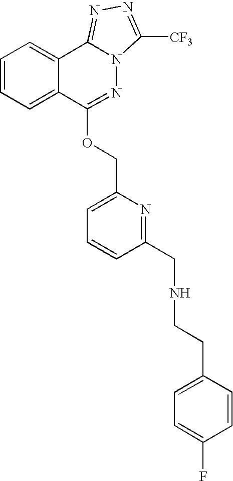

Utilizing the general procedure outlined in Example 5, 6-(3-trifluoromethyl-[1,2,4]triazolo[3,4-a]phthalazin-6-yloxymethyl)-pyridine-2-carbaldehyde (0.1 g, 0.3 mmol), 4-fluorophenethylamine (0.1 g, 0.6 mmol), sodium trisacetoxyborohydride (0.1 g, 0.6 mmol) and dichloroethane (2 mL) was reacted to give [2(4fluoro-phenyl)-ethyl]-[6-(3-trifluoromethyl-[1,2,4]triazolo[3,4-a]phthalazin-6-yloxymethyl)-pyridin-2-ylmethyl]-amine as a white solid: ¹H NMR (500 MHz, CDCl₃) δ 8.71 (d, 1H), 8.35 (d, 1H), 8.03 (t, 1H), 7.90 (t, 1H), 7.76 (t, 1H), 7.51 (d, 1H), 7.32 (d, 1H), 7.19 (t, 2H), 6.99 (t, 2H), 5.69 (s, 2H), 3.99 (s, 2H), 2.95 (t, 2H), 2.85 (t, 2H).

### Example 14

### (rac)-(2-Phenyl-propyl)-[6-(3-trifluoromethyl-[1,2,4]triazolo[3,4-a]phthalazin-6-yloxymethyl)-pyridin-2-ylmethyl]-amine

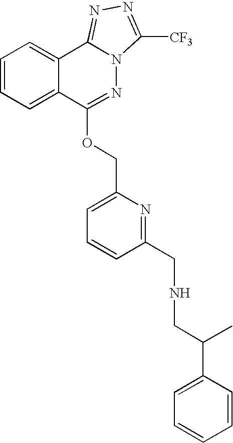

Utilizing the general procedure outlined in Example 5, 6-(3-trifluoromethyl-[1,2,4]triazolo[3,4-a]phthalazin-6-yloxymethyl)-pyridine-2-carbaldehyde, 2-phenyl-propylamine, sodium trisacetoxyborohydride and dichloroethane (2 mL) was reacted to give (rac)-(2-phenyl-propyl)-[6-3-trifluoromethyl-[1,2,4]triazolo[3,4-a]phthalazin-6-yloxymethyl)-pyridin-2-ylmethyl]-amine as a white solid: ¹H NMR (DMSO-d₆, 500 MHz) 8.74 (d, 1 H), 8.31 (d, 1 H), 8.05 (m, 1 H), 7.95 (t, 1 H), 7.81 (t, 1 H), 7.51 (d, 1 H), 7.37 (m, 1 H), 7.28 (m, 5 H), 5.46 (m, 2 H), 4.28 (m, 1 H), 4.20 (d, 1H), 3.21 (m, 2H), 2.8 (m, 1H), 2.8 (m, 1H); LCMS (ESI) m/z 493 (493 calcd for C₂₆H₂₄F₃N₆O M+H).

### Example 15

### (rac)-(1-Methyl-2-phenyl-ethyl)-[6-(3-trifluoromethyl-[1,2,4]triazolo[3,4-a]phthalazin-6-yloxymethyl)-pyridin-2-ylmethyl]-amine

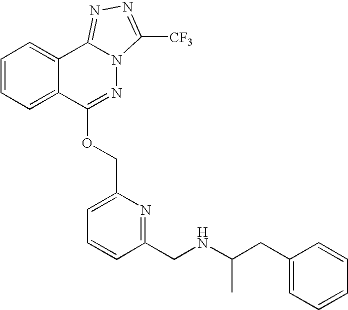

This compound was prepared as in Example 5 by replacing 3-methylphenethylamine with dl-□ methylphenethylamine to afford (rac)-(1-methyl-2-phenyl-ethyl)-[6-(3-trifluoromethyl-[1,2,4]triazolo[3,4-a]phthalazin-6-yloxymethyl)-pyridin-2-ylmethyl]-amine as a colorless oil; ¹H NMR (500 MHz, CDCl₃) δ 8.64 (d, 1H), 8.30 (d, 1H), 7.99-7.95 (m, 1H), 7.87-7.83 (m, 1H), 7.71 (t, 1H), 7.46 (d, 1H), 7.28-7.16 (m, 6H), 5.60 (s, 2H), 4.00 (d, 1H), 3.93 (d, 1H), 2.97-2.93 (m, 1H), 2.82-2.78 (m, 1H), 2.67-2.63 (m, 1H), 2.30 (br s, 1H), 1.11(s, 3H); LRMS (ESI) m/z 493 (493 calcd for C₂₆H₂₄F₃N₆O, M+H).

### Example 16

### (S)-1-Methyl-2-phenyl-ethyl)-[6-(3-trifluoromethyl-[1,2,4]triazolo[3,4-a]phthalazin-6-yloxymethyl)-pyridin-2-ylmethyl]-amine

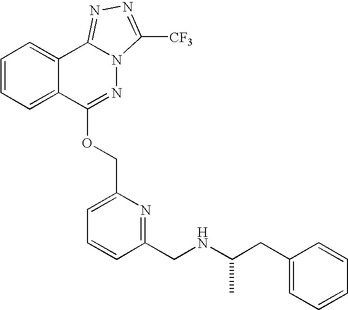

This compound was prepared as in Example 5 by replacing 3-methylphenethylamine with d-(+)-□ methylphenethylamine to provide (S)-1-Methyl-2-phenyl-ethyl)-[6-(3-trifluoromethyl-[1,2,4]triazolo[3,4-a]phthalazin-6-yloxymethyl)-pyridin-2-ylmethyl]-amine as a colorless oil; ¹H NMR (500 MHz, CDCl₃) δ 8.64 (d, 1H), 8.30 (d, 1H), 7.99-7.95 (m, 1H), 7.87-7.83 (m, 1H), 7.71 (t, 1H), 7.46 (d, 1H), 7.28-7.16 (m, 6H), 5.60 (s, 2H), 4.00 (d, 1H), 3.93 (d, 1H), 2.97-2.93 (m, 1H), 2.82-2.78 (m, 1H), 2.67-2.63 (m, 1H), 2.30 (br s, 1H), 1.11 (s, 3H); LRMS (ESI) m/z 493 (493 calcd for C₂₆H₂₄F₃N₆O, M+H).

### Example 17

### (rac)-(trans-2-Phenyl-cyclopropyl)-[6-(3-trifluoromethyl-[1,2,4]triazolo[3,4-a]phthalazin-6-yloxymethyl)-pyridin-2-ylmethyl]-amine

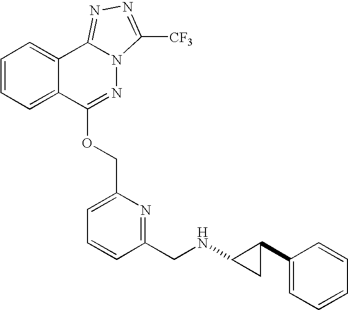

This compound was prepared as in Example 5 by replacing 3-methylphenethylamine with trans-2-phenyl-cyclopropyl amine to afford (rac)-(trans-2-Phenyl-cyclopropyl)-[6-(3-trifluoromethyl-[1,2,4]triazolo[3,4-a]phthalazin-6-yloxymethyl)-pyridin-2-ylmethyl]-amine as a colorless oil; ¹H NMR (500 MHz, CDCl₃) δ 8.66 (d, 1H), 8.32 (d, 1H), 8.00-7.97 (m, 1H), 7.88-7.85 (m, 1H), 7.74 (t, 1H), 7.51 (d, 1H), 7.29 (d, 1H), 7.23-7.08 (m, 3H), 6.99-6.97 (m, 2H), 5.67 (s, 2H), 4.06 (s, 2H), 2.53 (br s, 1H), 2.43-2.40 (m, 1H), 1.96-1.93 (m, 1H), 1.16-1.12 (m, 1H), 1.00-0.97 (m, 1H); LRMS (ESI) m/z 491 (491 calcd for C₂₆H₂₂F₃N₆O, M+H).

### Example 18

### (rac)-(2-in-Tolyl-ethyl)-{1-[6-(3-trifluoromethyl-[1,2,4]triazolo[3,4-a]phthalazin-6-yloxymethyl)-pyridin-2-yl]-ethyl}-amine

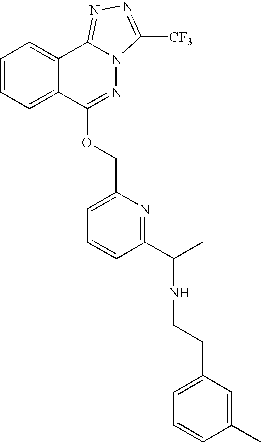

### Step 1: Synthesis of (rac)-1-[6-(3-Trifluoromethyl-[1,2,4]triazolo[3,4-a]phthalazin-6-yloxymethyl)-pyridin-2-yl]ethanol

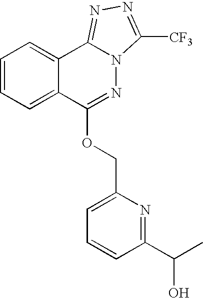

A THF solution of methylmagnesium bromide (0.8 mL, 1.0 M) was added dropwise to [6-(3-trifluoromethyl-[1,2,4]triazolo[3,4-a]phthalazin-6-yloxymethyl)-pyridin-2-yl]-methanol (0.3 g, 0.8 mmol) and THF (0.5 mL) at 0° C. The mixture was stirred for 10 minutes, allowed to warm to room temperature, then heated to 60° C. After 15 hours, the reaction mixture was quenched with NaHCO₃ (1 mL), concentrated and dissolved in EtOAc (30 mL). The organic layer was washed with water (1×10 mL), brine (2×10 mL). The combined organic layers were dried over MgSO₄, filtered, and concentrated. The residue purified on silica gel (1:1 ethyl acetate-hexanes) to afford (rac)-1-[6-(3-trifluoromethyl-[1,2,4]triazolo[3,4-a]phthalazin-6-yloxymethyl)-pyridin-2-yl]-ethanol as a white solid: ¹H NMR (500 MHz, CDCl₃) δ 8.69 (d, 1H), 8.34 (d, 1H), 8.00 (t, 1H), 7.90 (t, 1H), 7.81 (t, 1H), 7.51 (d, 1H), 7.30 (d, 1H), 5.71 (s, 2H), 4.93 (m, 1H), 1.53 (d, 3H).

### Step 2: Synthesis of 1-[6-(3-trifluoromethyl-[1,2,4]triazolo[3,4-a]phthalazin-6-yloxymethyl)-pyridin-2-yl]-ethanone

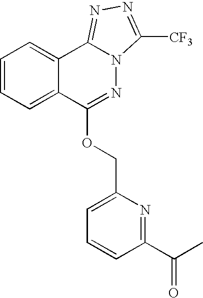

To 1-[6-(3-trifluoromethyl-[1,2,4]triazolo[3,4-a]phthalazin-6-yloxymethyl)-pyridin-2-yl]-ethanol (0.15 g, 0.37 mmol) in dichloromethane (18 mL) was added Dess-Martin periodinane (0.24 g, 0.56 mmol) (J. Org. Chem. 1983, 48, 4155). The mixture was stirred for 3 hours while monitoring by TLC. The reaction mixture was diluted with CH₂Cl₂ (30 mL) and extracted with 1 M NaOH (2×20 mL). The combined organic layers was dried over MgSO₄, filtered, and concentrated to afford 1-[6-(3-trifluoromethyl-[1,2,4]triazolo[3,4-a]phthalazin-6-yloxymethyl)-pyridin-2-yl]-ethanone as a white solid: LCMS (ESI) m/z 388 (388 calcd for C₁₈H₁₂F₃N₅O₂ M+H).

### Step 3: Synthesis of (rac)-(2-m-tolyl-ethyl)-{1-[6-(3-trifluoromethyl-[1,2,4]triazolo[3,4-a]phthalazin-6-yloxymethyl)-pyridin-2-yl]-ethyl}-amine

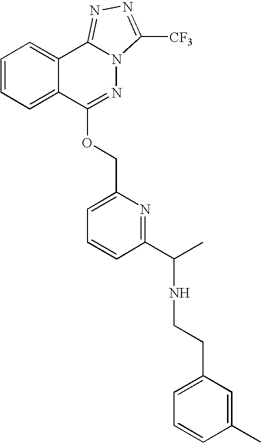

Utilizing the general procedure outlined in Step 2 of Example 5, 1-[6-(3-trifluoromethyl-[1,2,4]triazolo[3,4-a]phthalazin-6-yloxymethyl)-pyridin-2-yl]-ethanone (0.1 g, 0.3 mmol), 3-methylphenethylamine (0.1 g, 0.6 mmol), sodium trisacetoxyborohydride (0.1 g, 0.6 mmol) and THF (3.2 mL) reacted to give (rac)-(2-m-tolyl-ethyl)-{1-[6-(3-trifluoromethyl-[1,2,4]triazolo[3,4-a]phthalazin-6-yloxymethyl)-pyridin-2-yl]-ethyl}-amine as a white solid: ¹H NMR (500 MHz, CDCl₃) δ 8.71 (d, 1H), 8.30 (d, 1H), 8.03 (t, 1H), 7.9 (m, 2H), 7.63(m, 2H), 7.21 (t, 1H), 7.05 (d, 2H), 6.96 (d, 1H ), 5.54 (s, 2H), 4.61 (s, 1H), 3.13 (m, 3H), 3.0 (s, 1H), 2.25 (s, 3H), 1.91 (bs, 3H).

### Example 19

### (rac)-2-Methyl-{1-[6-(3-trifluoromethyl-[1,2,4]triazolo[3,4-a]phthalazin-6-yloxymethyl)-pyridin-2-yl]-propyl}-phenethyl-amine

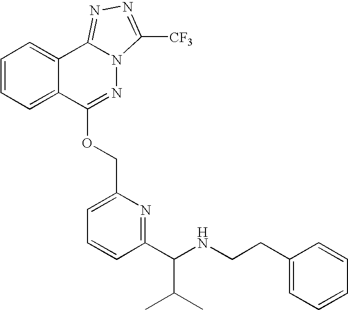

This compound was prepared as in Example 18 replacing methyl magnesium bromide with isopropyl magnesium bromide in Step 1 to afford (rac)-2-methyl-{1-[6-(3-trifluoromethyl-[1,2,4]triazolo[3,4-a]phthalazin-6-yloxymethyl)-pyridin-2-yl]-propyl}-phenethyl-amine as a colorless oil: ¹H NMR (500 MHz, CDCl₃) δ 8,64 (d, 1H), 8.31 (d, 1H), 7.98-7.95 (m, 1H), 7.86-7.83 (m, 1H), 7.72-7.68 (m, 1H), 7.43 (d, 1H), 7.24-7.10 (m, 6H), 5.65 (s, 2H), 3.50 (d, 1H), 2.76-2.63 (m, 4H), 1.98-1.94 (M, 1H), 0.90 (d, 3H), 0.73 (d, 3H); LRMS (ESI) m/z 521 (521 calcd for C₂₈H₂₈F₃N₆O, M+H).

### Example 20

### (rac)-Phenethyl-{1-[6-(3-trifluoromethyl-[1,2,4]triazolo[3,4-a]phthalazin-6-yloxymethyl)-pyridin-2-yl]-propyl}-amine

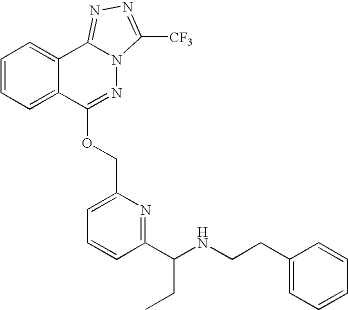

This compound was prepared as in Example 18 replacing methyl magnesium bromide with ethyl magnesium bromide in Step 1 to afford (rac)-phenethyl-{1-[6-(3-trifluoromethyl-[1,2,4]triazolo[3,4-a]phthalazin-6-yloxymethyl)-pyridin-2-yl]-propyl}-amine as a colorless oil: ¹H NMR (500 MHz, CDCl₃) δ 8.55 (d, 1H), 8.25 (d, 1H), 7.92 (t, 1H), 7.80 (t, 1H), 7.70 (t, 1H), 7.45 (d, 1H), 7.23 (d, 1H), 7.20-7.07 (m, 5H), 5.61 (s, 2M, 3.67 (t, 1H), 2.77-2.62 (m, 4H), 1.75-1.68 (m, 2H), 0.77 (t, 3H); LRMS (ESI) m/z 507 (507 calcd for C₂₇H₂₆F₃N₆O, M+H).

### Example 21

### Phenethyl-[5-(3-trifluoromethyl-[1,2,4]triazolo[3,4-a]phthalazin-6-yloxymethyl)-pyridin-3-ylmethyl]-amine

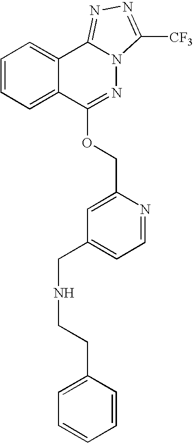

### Step 1: Synthesis of 5-(tert-Butyl-dimethyl-silanyloxymethyl)-pyridine-3-carbaldehyde

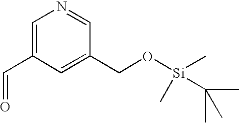

A hexane solution of n-BuLi (0.9 mL, 1.6 M) was added slowly to 3-bromo-5-(tert-butyl-dimethyl-silanyloxymethyl)-pyridine (J. Med. Chem. 1997, 40, 2866) (0.4 g, 1.4 mmol) in THF (6 mL) at −78° C. After 1 hour, anhydrous DMF (0.45 mL) was added and the mixture was allowed to warm to 0° C. and stirred for 90 minutes. Saturated aqueous NaHCO₃ (5 mL) was added and the mixture extracted with CH₂Cl₂ (3×10 mL). The combined organic layers were dried over MgSO₄, filtered and concentrated. Purification was performed on silica gel (hexanes-ethyl acetate 1:1) to afford 5-(tert-Butyl-dimethyl-silanyloxymethyl)-pyridine-3-carbaldehyde as a colorless oil: ¹H NMR (500 MHz, CDCl₃) δ 10.14 (s, 1H), 8.97 (s, 1H), 8.81 (s, 1H), 8.13 (s, 1H), 4.75 (s, 2H), 0.97 (s, 9H), 0.15 (s, 6H).

### Step 2: Synthesis of 5-(tert-butyl-dimethyl-silanyloxymethyl)-pyridin-3-ylmethyl]-phenethyl-amine

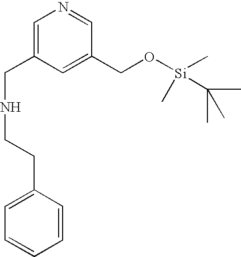

To 5-(tert-butyl dimethyl-silanyloxymethyl)-pyridine-3-carbaldehyde (0.19 g, 0.756 mmol) and phenethylamine (0.18 g, 1.51 mmol) in dichloroethane (5 mL) was added sodium triacetoxyborohydride (NaBH(OAc)₃) (0.32 g, 1.51 mmol) in one portion. The mixture was stirred at rt for 15 hours, diluted with CH₂Cl₂ (20 mL) and extracted with 1 M NaOH (1×20 mL). The organic layer was dried over MgSO₄, filtered and concentrated. Purification was performed on silica gel (1:19 MeOH—CH₂Cl₂) to provide 5-(tert-butyl-dimethyl-silanyloxymethyl)-pyridin-3-ylmethyl]-phenethyl-amine as a colorless oil: ¹H NMR (500 MHz, CDCl₃) δ 8.50 (d, 2H), 7.60 (s, 1H), 7.35 (m, 2H), 7.25 (m, 3H), 4.75 (s, 2H), 3.85 (s, 2H), 2.90 (m, 2H), 2.8 (m, 2H), 0.95 (s, 9H), 0.15 (s, 6H).

### Step 3: Synthesis of (5-hydroxymethyl-pyridin-3-ylmethyl) -phenethyl-carbamic acid tert-butyl ester

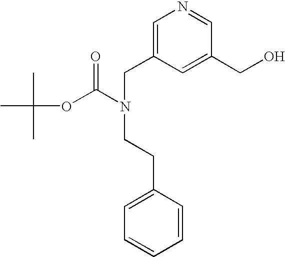

To a solution of 5-(tert-butyl-dimethyl-silanyloxymethyl)-pyridin-3-ylmethyl]-phenethyl-amine (0.16 g, 0.43 mmol) in CH₂Cl₂ (4.5 mL) was added di-tert-butyldicarbonate (0.19 g, 0.870 mmol). After stirring for 18 hours at rt, the mixture was diluted with CH₂Cl₂ (10 mL) and partitioned with NaHCO₃. The layers were separated and the organic layer extracted with water (1×10 mL). The combine organic layers were dried over MgSO₄, filtered, and concentrated. The crude residue obtained (0.17 g) was dissolved in THF (2.3 mL) followed by addition of tetrabutylammonium fluoride (0.57 mL, 1 M in THF) at rt. After 1 h saturated aqueous NaHCO₃ was added (3 mL) followed by removal of THF in vacuo. The concentrated mixture was extracted with CH₂Cl₂ (2×5 mL). The combined organic layers were extracted with water (2×5 ml), brine (1×5 mL) and dried over MgSO₄ to provide (5-hydroxymethyl-pyridin-3-ylmethyl) -phenethyl-carbamic acid tert-butyl ester which was used without further purification: LCMS (ESI) m/z 343 (343 calcd for C₂₀H₂₆N₂O₃, M+H).

### Step 4: Synthesis of phenethyl-[5-(3-trifluoromethyl-[1,2,4]triazolo[3,4-a]phthalazin-6-yloxymethyl)-pyridin-3-ylmethyl]-amine

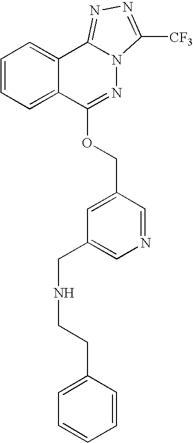

6-Chloro-3-trifluoromethyl-[1,2,4]triazolo[3,4-a]phthalazine (0.10 g, 0.36 mmol) and (5-hydroxymethyl-pyridin-3-ylmethyl) -phenethyl-carbamic acid tert-butyl ester (0.10 g, 0.29 mmol) were charged with DMF (2 mL) and cooled to −78° C. A THF solution of lithium bis(trimethysilyl) amide (0.33 mL, 1 M) was added dropwise to the mixture After 30 min, the reaction was quenched with saturated aqueous NaHCO₃ (2 mL) and diluted with CH₂Cl₂ (10 mL). The layers were separated and the organic layer extracted with water (5×5 mL) and brine (1×5 mL). The resulting solution was dried over MgSO₄, filtered and concentrated. Purification on silica gel (3-7% MeOH—CH₂Cl₂) provided a white foam. Removal of Boc group using 4N HCl in dioxane via standard conditions afforded phenethyl-[5-(3-trifluoromethyl-[1,2,4]triazolo[3,4-a]phthalazin-6-yloxymethyl)-pyridin-3-ylmethyl]-amine as a hydrochloride salt: ¹H NMR (500 MHz, CD₃OD) δ 9.26 (s, 1H), 9.13 (d, 2 H), 8.64 (d, 1H), 8.54 (d, 1H), 8.16 (t, 1H), 8.05 (t, 1H ), 7.35 (m, 4H), 7.27 (t, 1H), 5.91 (s, 2H), 4.60, (s, 2H), 3.45 (m, 2H), 3.12 (t, 2H).

### Example 22

### (rac)-2-({[6-({(3-(5-Methylisoxazol-3-yl)[1,2,4]triazolo[3, 4-a]phthalazin-6-yl)oxy}methyl)-pyridin-2-yl)methyl}amino)-1-phenylethanol

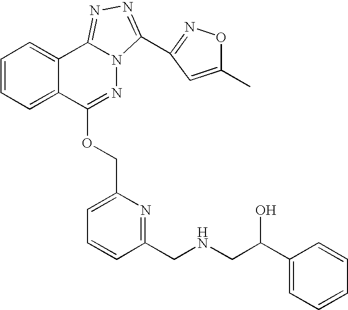

1,4-dichlorophthalazine (5.6 g, 28 mmol) and 5-methylisoxazole-3-carbohydrazine (J. Heterocycl. Chem. 1992, 29, 1101) (4.0 g, 28 mmol) were dissolved in dioxane (100 mL) and heated to reflux for 12 h. The reaction mixture was concentrated, dissolved in CH₂Cl₂, washed with a saturated aqueous sodium bicarbonate solution, dried (MgSO₄) to afford the desired triazolophthalazine. The triazolophthalazine (570 mg, 2.0 mmol) and [6-({tert-butyl-dimethyl)silyl]oxy}methyl)pyridin-2-yl]methanol (J. Org. Chem. 1993, 58, 4389) (506 mg, 2.0 mmol) were dissolved in DMF (4.0 mL), cooled to −78° C. and treated with a solution of lithium bis(trimethylsilyl)amide (2.0 mL of 1 M in THF). The reaction was allowed to warm to room temperature over 12 h, concentrated, and the crude product was dissolved in THF (1 mL) and treated with TBAF (2.0 mL of 1 M in THF). After stirring the reaction for 2 h at ambient temperature, the product precipitated from solution, was filtered and dried to afford the desired alcohol. The alcohol (170 mg, 0.44 mmol) was dissolved in CH₂Cl₂ (2 mL) and treated with triethylamine (0.092 mL, 0.66 mmol) and methanesulfonyl chloride (0.044 mL, 0.57 mmol). The reaction was stirred at ambient temperature for 1 h, diluted with CH₂Cl₂ (5 mL) and washed with a saturated aqueous sodium bicarbonate solution, dried (MgSO₄), and concentrated to afford the mesylate. The crude mesylate residue was dissolved in methylene chloride (2 mL) and treated with 2-amino-1-phenylethanol (110 mg, 0.8 mmol) at ambient temperature for 20 h. The reaction was concentrated, and the residue was purified by flash column chromatography (SiO₂, 1%-20% MeOH in CH₂Cl₂) to afford (rac)-2-({[6-({(3-(5-methylisoxazol-3-yl)[1,2,4]triazolo[3,4-a]phthalazin-6-yl)oxy}methyl)pyridin-2-yl)methyl)}amino)-1-phenylethanol: ¹H NMR (500 MHz, CDCl₃) δ 8.68 (d, 1H ), 8.31 (d, 1H), 7.96 (dt, 1H), 7.81 (dt, 1H), 7.76 (t, 1H), 7.65 (d, 1H), 7.33 (m, 6H), 6.86 (m, 1H), 5.75 (s, 2H), 5.32 (s, 1H), 4.80 (dd, 1H), 4.06 (s, 2H), 3.00 (dd, 1H), 2.84 (dd, 1H), 2.58 (s, 3H); LCMS (ESI) m/z 507 (507 calcd for C₂₈H₂₅N₇O₃, M+H).

### Example 23

### N-[(6-{[(3-Methyl[1,2,4[triazolo[3,4-a]phthalzin-6-yl)oxy]methyl}pyridin-2-yl)methyl]-2-phenylethanamine

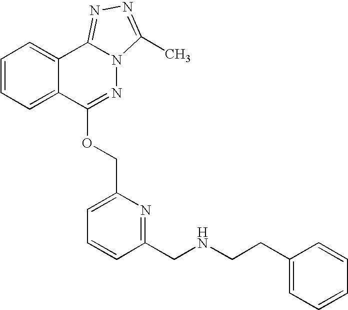

1-chloro-4-hydrazinophthalazine hydrochloride (Helv. Chim. Acta. 1951, 34, 195) (0.5 g, 2.6 mmol), triethylamine (0.43 mL, 3.1 mmol) and acetyl chloride (0.22 mL, 3.1 mmol) were dissolved in dioxane (9 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂ to afford the desired triazolophthalazine. The triazolophthalazine (300 mg, 1.4 mmol) and [6-({tert-butyl-dimethyl)silyl]oxy)methyl)pyridin-2-yl]methanol (J. Org. Chem. 1993, 58, 4389) (230 mg, 1.4 mmol) were dissolved in DMF (1.5 mL), cooled to −78° C. and treated with a solution of lithium bis(trimethylsilyl)amide (1.4 mL of 1 M in THF). The reaction was allowed to warm to room temperature over 12 h, concentrated, and the crude product was dissolved in THF (1 mL) and treated with TBAF (1.4 mL of 1 M in THF). After stirring the reaction for 2 h at ambient temperature, the reaction was concentrated and purified by flash column chromatography (SiO₂, 1 to 9% MeOH in CH₂Cl₂) to afford the desired alcohol. The alcohol (90 mg, 0.28 mmol) was dissolved in CH₂Cl₂ (1 mL) and treated with triethylamine (0.078 mL, 0.56 mmol) and methanesulfonyl chloride (0.034 mL, 0.42 mmol). The reaction was stirred at ambient temperature for 1 h, diluted with CH₂Cl₂ (5 mL) and washed with a saturated aqueous sodium bicarbonate solution, dried (MgSO₄), and concentrated to afford the mesylate. The crude mesylate residue was dissolved in methylene chloride (1 mL) and treated with phenethylamine (170 mg, 1.4 mmol) at ambient temperature for 20 h. The reaction was concentrated, and the residue was purified by flash column chromatography (SiO₂, 1-20% MeOH in CH₂Cl₂) to afford N-[(6-{[(3-methyl[1,2,4]triazolo[3,4-a]phthalzin-6-yl)oxy]methyl}pyridin-2-yl)methyl]-2-phenylethanamine: ¹H NMR (500 MHz, CDCl₃) δ 8.61 (d, 1H), 8.27 (d, 1H), 7.93 (dt, 1H), 7.77 (dt, 1H), 7.75 (t, 1H), 7.48 (d, 1H), 7.31 (m, 3H), 7.21 (m, 3H), 5.67 (s, 2H), 4.01 (s, 2H), 3.01 (s, 2H), 2.89 (t, 2H), 2.74 (s, 3H); LCMS (ESI) m/z 424 (424 calcd for C₂₅H₂₄N₆O, M+H).

### Example 24

### N-[(6-{[(3-Ethyl[1,2,4]triazolo[3,4-a]phthalzin-6-yl)oxy)methyl}pyridin-2-yl)methyl)-2-phenylethanamine

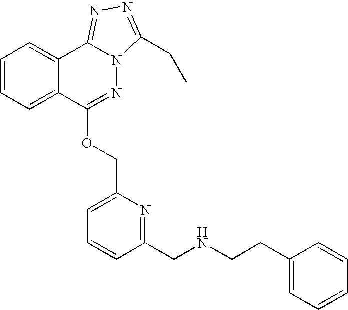

1-chloro-4-hydrazinophthalazine hydrochloride (Helv. Chim. Acta. 1951, 34, 195) (1.0 g, 4.3 mmol), triethylamine (0.72 mL, 5.2 mmol) and propionyl chloride (0.45 mL, 5.2 mmol) were dissolved in dioxane (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5%, MeOH in CH₂Cl₂ to afford the desired triazolophthalazine. The triazolophthalazine (0.6 g, 2.6 mmol) and [6-({tert-butyl-dimethyl)silyl]oxy}methyl)pyridin-2-yl]methanol (J. Org. Chem. 1993, 58, 4389) (654 mg, 2.6 mmol) were dissolved in DMF (10 mL), cooled to −78° C. and treated with a solution of lithium bis(trimethylsilyl)amide (2.6 mL of 1 M in THF). The reaction was allowed to warm to room temperature over 12 h, concentrated, and the crude product was dissolved in THF (1 mL) and treated with TBAF (2.6 mL of 1 M in THF). After stirring the reaction for 2 h at ambient temperature, the reaction was concentrated and purified by flash column chromatography (SiO₂, 1 to 9% MeOH in CH₂Cl₂) to afford the desired alcohol. The alcohol (335 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-ethyl[1,2,4]triazolo[3,4-a]phthalzin-6-yl)oxy]methyl}pyridin-2-yl)methyl]-2-phenylethanamine: ¹H NMR (500 MHz, CDCl₃) δ 8.61 (d, 1H), 8.27 (d, 1H), 7.92 (dt, 1H), 7.77 (dt, 1H), 7.77 (t, 1H), 7.46 (s, 1H), 7.31 (m, 3H), 7.22 (m, 3H), 5.66 (s, 2H), 4.00 (s, 2H), 3.15 (q, 2H), 2.99 (t, 2H), 2.89 (t, 2H), 1.45 (t, 3H); LCMS (ESI) m/z 439 (439 calcd for C₂₆H₂₆N₆O, M+H).

### Example 25

### N-[(6-{[(3-isopropyl[1,2,4]triazolo[3,4-a]phthalain-6-yl)oxy]methyl}pyridin-2-yl)methyl]-2-phenylethanamine

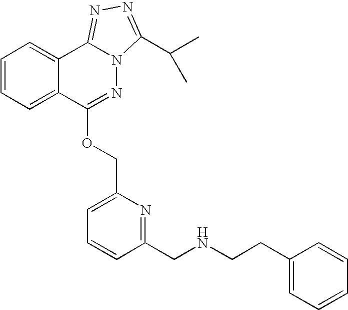

1-chloro-4-hydrazinophthalazine hydrochloride (Helv. Chim. Acta. 1951, 34, 195) (0.75 g, 3.3 mmol), triethylamine (0.54 mL, 3.9 mmol) and isobutyryl chloride (0.41 mL, 3.9 mmol) were dissolved in ethylene glycol dimethyl ether (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.5 g, 2.0 mmol) and [6-({tert-butyl-dimethyl)silyl]oxy}methyl)pyridin-2-yl]methanol (J. Org. Chem. 1993, 58, 4389) (510 mg, 2.0 mmol) were dissolved in DMF (10 mL), cooled to −78° C. and treated with a solution of lithium bis(trimethylsilyl)amide (2.0 mL of 1 M in THF). The reaction was allowed to warm to room temperature over 12 h, concentrated, and the crude product was dissolved in THF (3 mL) and treated with TBAF (3.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (349 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-isopropyl[1,2,4]triazolo[3,4-a]phthalain-6-yl)oxy]methyl}pyridin-2-yl)methyl]-2-phenylethanamine: ¹H NMR (500 MHz, (CDCl₃) δ 8.63 (d, 1H), 8.27 (d, 1H), 7.91 (t, 1H), 7.77 (t, 1H), 7.74 (t, 1H), 7.47 (d, 1H), 7.31 (m, 3H), 7.22 (m, 3H), 5.66 (s, 2H), 4.00 (s, 2H), 3.56 (septet, 1H), 2.98 (t, 2H), 2.89 (t, 2H), 1.51 (d, 6H); LCMS (ESI) m/z 453 (453 calcd for C₂₇H₂₈N₆O, M+H).

### Example 26

### N-[(6-{[(3-Cyclopropyl [1,2,4]triazolo[3,4-a]phthalain-6-yl)oxy]methyl}pyridin-2-yl)methyl]-2-phenylethanamine

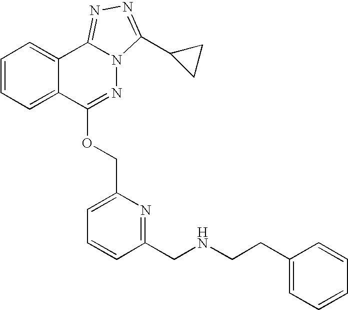

1-chloro-4-hydrazinophthalazine hydrochloride (Helv. Chim. Acta. 1951, 34, 195) (0.35 g, 3.9 mmol), triethylamine (0.54 mL, 3.9 mmol) and cyclopropanecarbonyl chloride (0.35 mL, 3.9 mmol) were dissolved in ethylene glycol dimethyl ether (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.75 g, 3.1 mmol) and [6-({tert-butyl-dimethyl)silyl]oxy}methyl)pyridin-2-yl]methanol (J. Org. Chem. 1993, 58, 4389) (780 mg, 3.1 mmol) were dissolved in DMF (10 mL), cooled to −78° C. and treated with a solution of lithium bis(trimethylsilyl)amide (3.1 mL of 1 M in THF). The reaction was allowed to warm to room temperature over 12 h, concentrated, and the crude product was dissolved in THF (4 mL) and treated with TBAF (4.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (347 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-cyclopropyl[1,2,4]triazolo[3,4-a]phthalain-6-yl)oxy]methyl}pyridin-2-yl)methyl]-2-phenylethanamine: ¹H NMR (500 MHz, CDCl₃) δ 8.59 (d, 1H), 8.25 (d, 1H), 7.90 (dt, 1H), 7.76 (m, 1H), 7.75 (m, 1H), 7.48 (d, 1H), 7.30 (m, 3H), 7.22 (m, 3H), 5.67 (s, 2H), 3.99 (s, 2H), 2.99 (t, 2H), 2.88 (m, 2H), 2.42 (m, 1H), 1.37 (m, 2H), 1.57 (m, 2H); LCMS (ESI) m/z 450 (450 calcd for C₂₇H₂₆N₆O, M+H).

### Example 27

### N-[(6-{[(3-Cyclobutyl[1,2,4]triazolo[3,4-a]phthalan-6-yl)oxy]methyl}pyridin-2-yl)methyl]-2-phenylethanamine

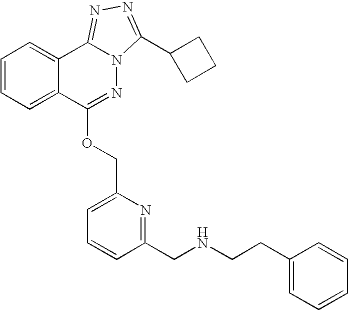

1-chloro-4-hydrazinophthalazine hydrochloride (Helv. Chim. Acta. 1951, 34, 195) (1.0 g, 4.3 mmol), triethylamine (0.72 mL, 5.2 mmol) and cyclobutanecarbonyl chloride (0.59 mL, 5.2 mmol) were dissolved in dioxane (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.68 g, 2.6 mmol) and [6-({tert-butyl-dimethyl)silyl]oxy}methyl)pyridin-2-yl]methanol (J. Org. Chem. 1993, 58, 4389) (670 mg, 2.6 mmol) were dissolved in DMF (10 mL), cooled to −78° C. and treated with a solution of lithium bis(trimethylsilyl)amide (2.6 mL of 1 M in THF). The reaction was allowed to warm to room temperature over 12 h, concentrated, and the crude product was dissolved in THF (3 mL) and treated with TBAF (3.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (361 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-cyclobutyl[1,2,4]triazolo[3,4-a]phthalain-6-yl)oxy]methyl}pyridin-2-yl)methyl]-2-phenylethanamine: ¹H NMR (500 MHz, CDCl₃) δ 8.61 (d, 1H), 8.25 (d, 1H), 7.90 (t, 1H), 7.75 (t, 1H), 7.73 (t, 1H), 7.45 (d, 1H), 7.30 (m, 3H), 7.22 (m, 3H), 5.64 (s, 2H), 4.04 (pentet, 1H), 3.99 (s, 2H), 2.96 (m, 2H), 2.89 (m, 2H), 2.67 (m, 2H), 2.47 (m, 2H), 2.16 (m, 1H), 2.09 (m, 1H); LCMS (ESI) m/z 464 (464 calcd for C₂₈H₂₈N₆O, M+H).

### Example 28

### N-[(6-{[(3-Cyclopentyl[1,2,4]triazolo[3,4-a]phthalain-6-yl)oxy]methyl}pyridin-2-yl)methyl]-2-phenylethanamine

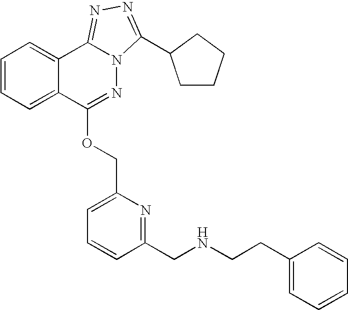

1-chloro-4-hydrazinophthalazine hydrochloride (Helv. Chim. Acta. 1951, 34, 195) (0.75 g, 3.3 mmol), triethylamine (0.54 mL, 3.9 mmol) and cyclopentanecarbonyl chloride (0.47 mL, 3.9 mmol) were dissolved in ethylene glycol dimethyl ether (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.75 g, 2.8 mmol) and [6-({tert-butyl-dimethyl)silyl]oxy}methyl)pyridin-2-yl]methanol (J. Org. Chem. 1993, 58, 4389) (700 mg, 2.8 mmol) were dissolved in DMF (10 mL), cooled to −78° C. and treated with a solution of lithium bis(trimethylsilyl)amide (2.8 mL of 1 M in THF). The reaction was allowed to warm to room temperature over 12 h, concentrated, and the crude product was dissolved in THF (4 mL) and treated with TBAF (4.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (375 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-cyclopentyl[1,2,4[triazolo[3,4-a]phthalain-6-yl)oxy]methyl}pyridin-2-yl)methyl]-2-phenylethanamine: ¹H NMR (500 MHz, CDCl₃) δ 8.61 (d, 1H), 8.25 (d, 1H), 7.91 (t, 1H), 7.74 (q, 2H), 7.45 (d, 1H), 7.31 (m, 3H), 7.21 (d, 3H), 5.64 (s, 2H), 3.99 (s, 2H), 3.61 (pentet, 1H), 2.97 (m, 2H), 3.88 (m, 2H), 2.18 (m, 2H), 2.07 (m, 2H), 1.90 (m, 2H), 1.75 (m, 2 H); LCMS (ESI) m/z 478 (478 calcd for C₂₉H₃₀N₆O, M+H).

### Example 29

### N-[(6-{[(3-Cyclohexyl[1,2,4]triazolo[3,4-a]phthalain-6-yl)oxy]methyl}pyridin-2-yl)methyl]-2-phenylethanamnine

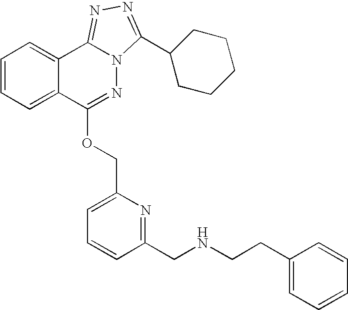

1-chloro-4-hydrazinophthalazine hydrochloride (Helv. Chim. Acta. 1951, 34, 195) (0.75 g, 3.3 mmol), triethylamine (0.54 mL, 3.9 mmol) and cyclohexanecarbonyl chloride (0.52 mL, 3.9 mmol) were dissolved in ethylene glycol dimethyl ether (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.70 g, 2.4 mmol) and [6-({tert-butyl-dimethyl)silyl]oxy}methyl)pyridin-2-yl]methanol (J. Org. Chem. 1993, 58, 4389) (620 mg, 2.4 mmol) were dissolved in DMF (10 mL), cooled to −78° C. and treated with a solution of lithium bis(trimethylsilyl)amide (2.4 mL of 1 M in THF). The reaction was allowed to warm to room temperature over 12 h, concentrated, and the crude product was dissolved in THF (3 mL) and treated with TBAF (3.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (389 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-cyclohexyl[1,2,4]triazolo[3,4-a]phthalain-6-yl)oxy]methyl}pyridin-2-yl)methyl]-2-phenylethanamine: ¹H NMR (500 MHz, CDCl₃) δ 8.63 (d, 1H), 8.27 (d, 1H), 7.91 (t, 1H), 7.76 (t, 1H), 7.73 (t, 1H), 7.45 (d, 1H), 7.30 (m, 3H), 7.22 (m, 3H), 5.66 (s, 2H), 3.99 (s, 2), 3.26 (m, 1H), 2.98 (t, 2H), 2.88 (m, 2H), 2.06 (m, 2H), 1.80 (m, 5H), 1.46 (m, 3H); LCMS (ESI) m/z 492 (492 calcd for C₃₀H₃₂N₆O, M+H).

### Example 30

### N-[(6-{[(3-Phenyl[1,2,4]triazolo[3,4-a]phthalain-6-yl)oxy]methyl}pyridin-2-yl)methyl]-2-phenylethanamine

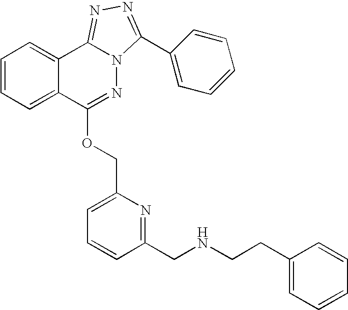

1-chloro-4-hydrazinophthalazine hydrochloride (Helv. Chim. Acta. 1951, 34, 195) (0.75 g, 3.3 mmol), triethylamine (0.54 mL, 3.9 mmol) and benzoyl chloride (0.45 mL, 3.9 mmol) were dissolved in ethylene glycol dimethyl ether (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.75 g, 2.7 mmol) and [6-{tert-butyl-dimethyl)silyl]oxy}methyl)pyridin-2-yl]methanol (J. Org. Chem. 1993, 58, 4389) (680 mg, 2.7 mmol) were dissolved in DMF (10 mL), cooled to −78° C. and treated with a solution of lithium bis(trimethylsilyl)amide (2.7 mL of 1 M in THF). The reaction was allowed to warm to room temperature over 12 h, concentrated, and the crude product was dissolved in THF (4 mL) and treated with TBAF (4.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (383 mg, 1.0 mmol) was dissolved in CH₂Cl₂ (10 mL) and treated with triethylamine (0.2 mL, 1.5 mmol) and methanesulfonyl chloride (0.1 mL, 1.25 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-phenyl[1,2,4]triazolo[3,4-a]phthalain-6-yl)oxy]methyl}pyridin-2-yl)methyl]-2-phenylethanamine: ¹H NMR (500 MHz, CDCl₃) δ 8.69 (d, 1H), 8.42 (d, 2H), 8.28 (d, 1H), 7.95 (t, 1H), 7.79 (t, 1H), 7.74 (t, 1H), 7.54 (m, 3H), 7.47 (m, 1H), 7.30 (m, 3H), 7.22 (m, 3H), 5.68 (s, 2H), 4.02 (m, 2H), 2.99 (m, 2H, 2.89 (m, 2H); LCMS (ESI) m/z 486 (486 calcd for C₃₀H₂₆N₆O, M+H).

### Example 31

### N-[(6-{[(3-(Methylthio)[1,2,4]triazolo[3,4-a]phthalain-6-yl)oxy]methyl}pyridin-2-yl)methyl]-2-phenylethanamine

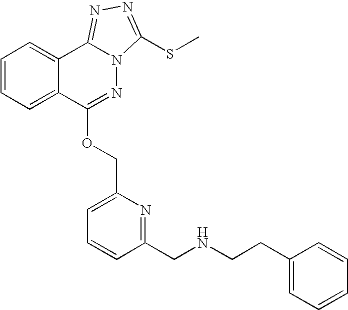

1-chloro-4-hydrazinophthalazine hydrochloride (Helv. Chim. Acta. 1951, 34, 195) (0.75 g, 3.3 mmol), potassium hydroxide (0.37 mg, 6.5 mmol) and carbon disulfide (0.39 mL, 6.5 mmol) were dissolved in ethanol (5 mL) and water (5 mL) and stirred for 12 h. The reaction mixture was concentrated, dissolved in sodium hydroxide (1 M) and filtered. The filtrate was acidified with hydrochloric acid (1 M) and the product precipitated from solution, was collected by filtration, and dried under vacuum. The sulfide (500 mg, 2.1 mmol) was dissolved in sodium hydroxide (2 mL of 1 M) and treated with methyl iodide (1 mL) at ambient temperature for 12 h. The product was extracted into CH₂Cl₂, dried (MgSO₄), and purified by flash column chromatography (SiO₂, 1%-15% MeOH in CH₂Cl₂) to afford the desired triazolophthalazine. The triazolophthalazine (0.25 g, 1.0 mmol) and [6-({tert-butyl-dimethyl)silyl]oxy}methyl)pyridin-2-yl]methanol (J. Org. Chem. 1993, 58, 4389) (250 mg, 1.1 mmol) were dissolved in DMF (5 mL), cooled to −78° C. and treated with a solution of lithium bis(trimethylsilyl)amide (1.1 mL of 1 M in THF). The reaction was allowed to warm to room temperature over 12 h, concentrated, and the crude product was dissolved in THF (4 mL) and treated with TBAF (1.5 mL of 1 M in THF). After stirring the reaction for 2 h at ambient temperature, the reaction was concentrated and purified by flash column chromatography (SiO₂, 1%-20% MeOH in CH₂Cl₂) to afford the desired alcohol. The alcohol (340 mg, 0.68 mmol) was dissolved in CH₂Cl₂ (5 mL) and treated with triethylamine (0.19 mL, 1.4 mmol) and methanesulfonyl chloride (0.08 mL, 1.0 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (3 mL) and treated with phenethylamine (0.43 mL, 3.4 mmol) at ambient temperature for 20 h. The reaction was concentrated, and the residue was purified by flash column chromatography (SiO₂, 1%-20% MeOH in CH₂Cl₂) to afford N-[(6-{[(3-(methylthio)[1,2,4]triazolo[3,4-a]phthalain-6-yl)oxy]methyl}pyridin-2-yl)methyl]-2-phenylethanamine: ¹H NMR (500 MHz, CDCl₃) δ 8.60 (d, 1H), 8.24 (d, 2H), 7.94 (dt, 1H), 7.79 (m, 1H), 7.52 (d, 1H), 7.32 (m, 3H), 7.25 (m, 3H), 5.59 (s, 2H), 4.10 (s, 2H), 3.11 (t, 2H), 3.01 (m, 2H), 2.84 (s, 3H); LCMS (ESI) m/z 456 (456 calcd for C₂₅H₂₄N₆OS, M+H).

### Example 32

### N-[(6-{[(3-(Methoxymethyl)[1,2,4]triazolo[3,4-a]phthalain-6-yl)oxy]methyl}pyridin-2-yl)methyl]-2-phenylethanamine

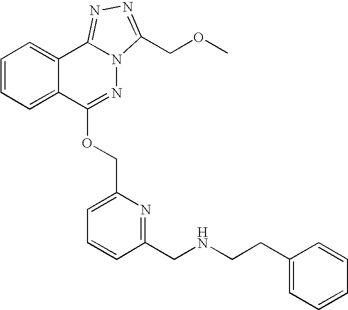

1-chloro-4-hydrazinophthalazine hydrochloride (Helv. Chim. Acta. 1951, 34, 195) (0.75 g, 3.3 mmol), triethylamine (0.54 mL, 3.9 mmol) and methoxyacetyl chloride (0.36 mL, 3.9 mmol) were dissolved in dioxane (10 mL) and heated to reflux for 12 h. The reaction mixture was concentrated, dissolved in CH₂Cl₂, washed with a saturated aqueous sodium bicarbonate solution, dried (MgSO₄), and purified by flash column chromatography (SiO₂, 1%-5% MeOH in CH₂Cl₂) to afford the desired triazolophthalazine. The triazolophthalazine (0.65 g, 2.6 mmol) and [6({tert-butyl-dimethyl)silyl]oxy}methyl)pyridin-2-yl]methanol (J. Org. Chem. 1993, 58, 4389) (660 mg, 2.6 mmol) were dissolved in DMF (10 mL), cooled to −78° C. and treated with a solution of lithium bis(trimethylsilyl)amide (2.6 mL of 1 M in THF). The reaction was allowed to warm to room temperature over 12 h, concentrated, and the crude product was dissolved in THF (3 mL) and treated with TBAF (3.0 mL of 1 M in THF). After stirring the reaction for 2 h at ambient temperature, the reaction was concentrated and purified by flash column chromatography (SiO₂, 1%-9% MeOH in CH₂Cl₂) to afford the desired alcohol. The alcohol (351 mg, 1.0 mmol) was dissolved in CH₂Cl₂ (10 mL) and treated with triethylamine (0.28 mL, 2.0 mmol) and methanesulfonyl chloride (0.11 mL, 1.5 mmol). The reaction was stirred at ambient temperature for 1 h, diluted with CH₂Cl₂ (20 mL) and washed with a saturated aqueous sodium bicarbonate solution, dried (MgSO₄), and concentrated to afford the mesylate. The crude mesylate residue was dissolved in methylene chloride (4 mL) and treated with phenethylamine (0.6 mL, 5.0 mmol) at ambient temperature for 20 h. The reaction was concentrated, and the residue was purified by flash column chromatography (SiO₂, 1%-20% MeOH in CH₂Cl₂) to afford N-[(6-{[(3-(methoxymethyl)[1,2,4]triazolo[3,4-a]phthalain-6-yl)oxy]methyl}pyridin-2-yl)methyl]-2-phenylethanamine: ¹H NMR (500 MHz, CDCl₃) δ 8.66 (d, 1H), 8.29 (d, 1H), 7.95 (t, 1H), 7.81 (t, 1H), 7.74 (t, 1H), 7.49 (t, 1H), 7.30 (m, 3H), 7.22 (m, 3H), 5.69 (s, 2H), 4.97 (s, 2H), 4.00 (s, 2H), 3.49 (s, 3H), 2.99 (t, 2H), 2.89 (t, 2H); LCMS (ESI) m/z 454 (454 calcd for C₂₆H₂₆N₆O₂, M+H).

### Example 33

### [6-(3-Methyl-7-phenyl-[1,2,4]triazolo[4,3-b]pyridazin-6-yloxymethyl)-pyridin-2-ylmethyl]-phenethyl-amine

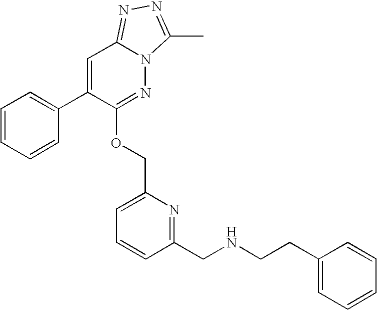

Utilizing the general procedure outlined in Example 23, 6-chloro-3-methyl-[1,2,4]triazolo[3,4-a]phthalazine was exchanged for 6-chloro-3-methyl-7-phenyl-[1,2,4]triazolo[4,3-b]pyridazine (Monatshefte fuer Chemie 1974, 105, 834) to provide [6-(3-Methyl-7-phenyl-[1,2,4]triazolo[4,3-b]pyridazin-6-yloxymethyl)-pyridin-2-ylmethyl]-phenethyl-amine as a white solid: ¹H NMR (500 MHz, DMSO-d₆) 7.97 (s, 1H), 7.63 (m, 3H), 7.52 (m, 3H), 7.30-7.07 (m, 7H), 5.63 (s, 2H), 4.03 (s, 2H), 3.14-2.81 (m, 4H), 2.74 (s, 3H); LRMS (ESI) m/z 451 (451 calcd for C₂₇H₂₇N₆O, M+H).

### Example 34

### 2-(4-Bromo-3-methylphenyl)-N-[3-({[3-(trifluoromethyl)[1,2,4]triazolo[3,4-a]phthalazin-6-yl]oxy}methyl)benzyl]ethanamine

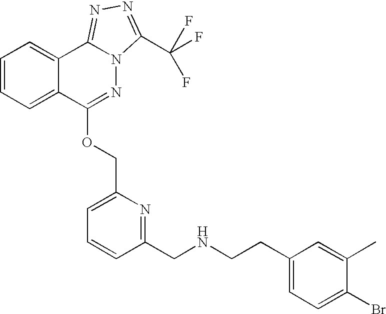

Utilizing the general procedure outlined in Example 4 [6-({[3-(trifluoromethyl)[1,2,4]triazolo[3,4-a]phthalazin-6-yl]oxy}methyl)pyridin-2-yl]methyl methanesulfonate (150 mg, 0.30 mmol) and 2-(4-bromo-3-methylphenyl)ethanamine (300 mg, 1.40 mmol) in DMF (1 ml) gave 2-(4-bromo-3-methylphenyl)-N-[3-({[3-(trifluoromethyl)[1,2,4]triazolo[3,4-a]phthalazin-6-yl]oxy{methyl)benzyl]ethanamine as a solid: ¹H NMR (500 MHz, CDCl₃) δ 8.88-8.87 (d, 1H), 8.32-8.31 (d, 1H), 8.01-7.98 (dt, 1H), 7.89-7.85 (dt, 1H), 7.75-7.72(t, 1H), 7.49-7.47 (d, 1H), 7.41-7.40 (d, 1H), 7.30-7.27 (m, 1H), 7.07 (m, 1H), 6.89-6.87 (m, 1H), 5.66 (s, 2H), 3.98 (s, 2H), 2.96-2.92 (m, 2H), 2.79-2.76 (m, 2H), 2.35 (s, 3H), 2.11 (br, 2H); LRMS (ESI) m/z 572 (572 calcd for C₂₆H₂₂BrF₃N₆, M+H).

### Example 35

### N-[3-({[3-(5-Methylisoxazol-3-yl)-7,8,9,10-tetrahydro[1,2,4]triazolo[3,4-a]phthalazin-6-yl]oxy}methyl)benzyl]-2-phenylethanamine

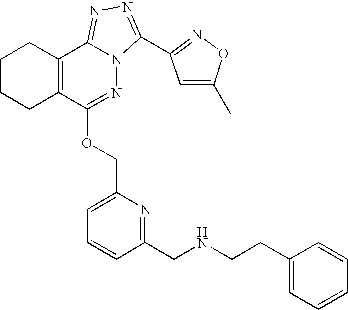

Utilizing the general procedure outlined in Example 22, 1,4-dichlorophthalazine was exchanged for 1,4-dichloro-5,6,7,8-tetrahydrophthalazine (J. Org. Chem. 1980, 45, 2320) to provide N-[3-([3-5-methylisoxazol-3-yl)-7,8,9,10-tetrahydro[1,2,4]triazolo[3,4-a]phthalazin-6-yl]oxy}methyl)benzyl]-2-phenylethanamine as a HCl salt: ¹H NMR (500 MHz, CD₃OD) δ 7.97-7.93 (m, 1H), 7.77-7.76 (d, 1H), 7.47-7.46 (d, 1H), 7.34-7.25 (m, 5H), 6.98 (s, 1H), 5.72 (s, 1H), 4.43 (s, 1H), 3.41-3.37 (m, 2H), 3.11-3.07 (m, 4H), 2.85 (m, 2H), 2.61 (s, 2H), 2.02-1.99 (m, 4H), 2.96-2.92 (m, 2H), 2.79-2.76 (m, 2H), 2.35 (s, 3H), 2.11 (br, 2H); LRMS (ESI) m/z 496 (496 calcd for C₂₈H₂₉N₇O₂, M+H).

### Example 36

### N-[3-({[3-(5-Methylisoxazol-3-yl)[1,2,4]triazolo[4,3-b]pyridazin-6-yl]oxy}methyl)benzyl]-2-phenylethanamine

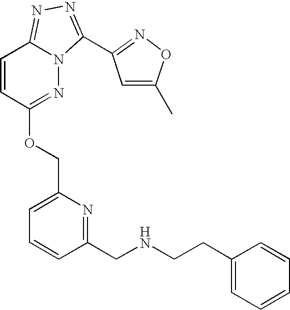

Utilizing the general procedure outlined in Example 22, 1,4-dichlorophthalazine was exchanged for 3,6-dichloropyridazine to provide N-[3-({[3-(5-methylisoxazol-3-yl)[1,2,4]triazolo[4,3-b]pyridazin-6-yl]oxy}methyl)benzyl]-2-phenylethanamine as a HCl salt: ¹H NMR (500 MHz, CD₃OD) δ 8.29-8.28 (d, 1H), 7.96-7.93 (t, 1H), 7.76-7.75 (d, 1H), 7.44-7.73 (d, 1H), 7.45-7.44 (d, 1H), 7.39-7.38 (d, 1H), 7.36-7.33 (m, 2H), 7.33-7.30 (m, 4H), 6.92 (m, 1H), 5.62 (s, 2H), 4.97 (s, 2H), 3.39-3.35 (m, 2H), 3.09-3.06 (m, 2H), 2.60 (s, 3H); LRMS (ESI) m/z 442 (442 calcd for C₂₄H₂₃N₇O₂, M+H).

### Example 37

### 3-(5-Methylisoxazol-3-yl)-6-[(3-{[(2-phenylethyl)amino]methyl}benzyl)oxy][1,2,4]triazolo[4,3-b]pyridazine-7-carboxamide

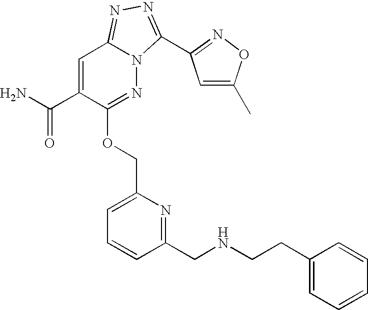

Utilizing the general procedure outlined in Example 22, 1,4-dichlorophthalazine was exchanged for 3,6-dichloropyridazine-4-carboxamide (J. Heterocyclic Chem. 1970, 7, 465) to provide 3-(5-methylisoxazol-3-yl)-6-[(3-{[(2-phenylethyl)amino]methyl}benzyl)oxy][1,2,4]-triazolo[4,3-b]pyridazine-7-carboxamide as a HCl salt: ¹H NMR (500 MHz, CD₃OD) δ 8.68 (s, 1H), 7.95-7.92 (t, 1H), 7.77-7.76 (d, 1H), 7.42-7.41 (d, 1H), 7.36-7.22 (m, 5H), 6.91 (s, 1H), 5.76 (s, 2H), 4.48 (s, 2H), 3.39-3.35 (m, 2H), 3.09-3.06 (m, 2H), 2.59 (s, 3H); LRMS (ESI) m/z 485 (485 calcd for C₂₅H₂₄N₈O₃, M+H).

### Example 38

### 3-(5-Methylisoxazol-3-yl)-6-[(3-{[(2-phenylethyl)amino]methyl}benzyl)oxy][1,2,4]triazolo[4,3-b]pyridazine-8-carboxamide

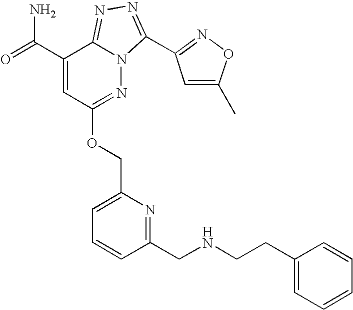

Utilizing the general procedure outlined in Example 22, 1,4-dichlorophthalazine was exchanged for 3,6-dichloropyridazine-4-carboxamide (J. Heterocyclic Chem. 1970, 7, 465) to afford 3-(5-methylisoxazol-3-yl)-6-[(3-{[(2-phenylethyl)amino]methyl}benzyl)oxy][1,2,4]triazolo[4,3-b]pyridazine-8-carboxamide as a HCl salt: ¹H NMR (500 MHz, CD₃OD) δ 7.97-7.94 (t, 1H), 7.78-7.76 (m, 2H), 7.45-7.44 (d, 1H), 7.36-7.23 (m, 6H), 6.94 (s, 1H), 5.70 (s, 2H), 4.56 (s, 2H), 3.41-3.37 (m, 2H), 3.01-3.03 (m, 2H), 2.59 (s, 3H); LRMS (ESI) m/z 485 (485 calcd for C₂₅H₂₄N₈O₃, M+H).

### Example 39

### N-[3-({[8-Methyl-3-(5-methylisoxazol-3-yl)[1,2,4]triazolo[4,3-b]pyridazin-6-yl]oxy}methyl)benzyl]-2-phenylethanamine and N-[3-({[7-Methyl-3-(5-methylisoxazol-3-yl)[1,2,4]triazolo[4,3-b]pyridazin-6-yl]oxy}methyl)benzyl]-2-phenylethanamine

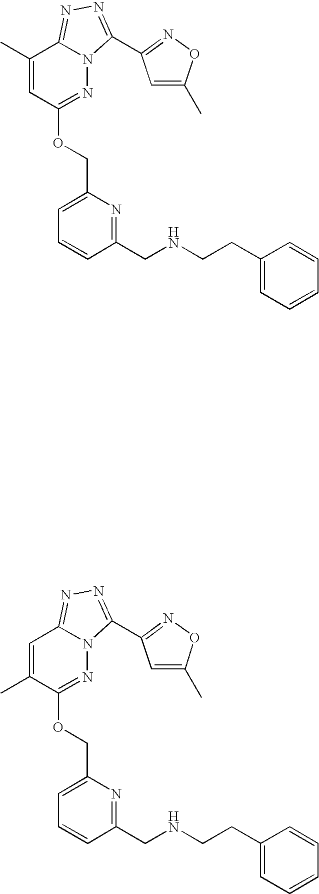

Utilizing the general procedure outlined in Example 22, 1,4-dichlorophthalazine was exchanged for 3,6-dichloro-4-methylpyridazine to give N-[3-({[8-methyl-3-(5-methylisoxazol-3-yl)[1,2,4]triazolo[4,3-b]pyridazin-6-yl]oxy}methyl)benzyl]-2-phenylethanamine and N-[3-({[7-methyl-3-(5-methylisoxazol-3-yl)[1,2,4]triazolo[4,3-b]pyridazin-6-yl]oxy}methyl)benzyl]-2-phenylethanamine as an inseparable mixture of isomers by silica gel chromatography as a yellow oil: ¹H NMR (500 MHz, CD₃OD, 2:1 mixture of isomers). δ 8.46 (br, 2H), 8.13-8.11 (m, 3H), 7.93-7.89 (m, 3H), 7.68-7.66 (m, 3H), 7.60 (m, 1H), 7.35-7.31 (m, 12H), 7.27-7.23 (m, 3H), 7.06-7.04 (m, 3H), 5.84 (s, 4H), 5.79 (s, 2H), 4.56 (s, 6H), 3.42-3.40 (m, 6H), 3.14-3.10 (m, 6H), 2.63-2.61 (m, 16H); LRMS (ESI) m/z 456 (456 calcd for C₂₅H₂₅N₇O₂, M+H).

### Example 40

### 2-Phenyl-N-[(6-{[(4-pyridin-3-ylphthalazin-1-yl)oxy]methyl}pyridin-2-yl)methyl]ethanamine

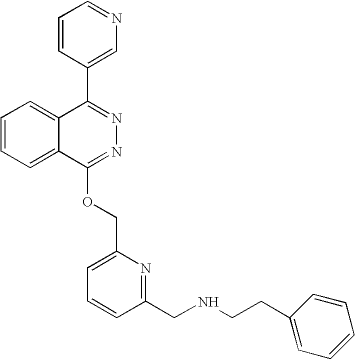

### Step 1: Synthesis of 1-{[6-({[tert-butyl(dimethyl)silyl]oxy}methyl)pyridin-2-yl]methoxy}-4-chlorophthalazine

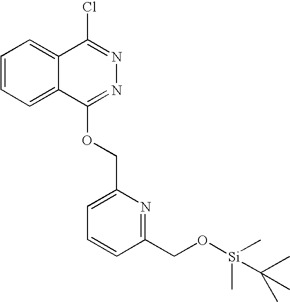

A THF solution of sodium bis(trimethylsilyl)amide (7.1 mL, 1.0 M) was added dropwise via syringe pump to a flask containing 1,4-dichorophthalazine (1.4 g, 7.1 mmol) and [6-({[tert-butyl(dimethyl)silyl]oxy}methyl)pyridin-2-yl]methanol (1.8 g, 7.1 mmol) (J. Org. Chem. 1993, 58, 4389) in THF: DMF (30 mL, 2:1) at −78° C. under N₂. The mixture was slowly warned to rt and continued stirring overnight. The mixture was partitioned between saturated aqueous NaHCO₃ (20 mL), and EtOAc (60 mL). The phases were separated and the aqueous layer was extracted with EtOAc (3×60 mL). The combined organic layers were dried (MgSO₄), filtered, and concentrated. The residue was purified on silica gel (20:1 hexane-ethyl acetate) to afford 1-{[6-({[tert-butyl(dimethyl)silyl]oxy}methyl)pyridin-2-yl]methoxy}-4-chlorophthalazine as a yellow solid: LRMS (ESI) m/z 416 (416 calcd for C₂₁H₂₆ClN₃O₂Si, M+H).

### Step 2: Synthesis of (6-{[(4-chlorophthalazin-1-yl)oxy]methyl}pyridin-2-yl)methanol

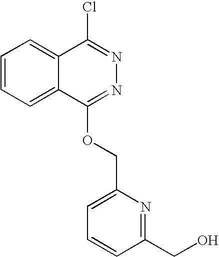

A THF solution of TBAF (1.2 mL, 1.0 M) was added dropwise to 1-{[6-({[tert-butyl(dimethyl)silyl]oxy}methyl)pyridin-2-yl]methoxy}-4-chlorophthalazine (480 mg, 1.15 mmol) in THF (10 mL). After 30 min, the mixture was partitioned between saturated aqueous NaHCO₃ (10 mL), and EtOAc (20 mL). The phases were separated and the aqueous layer was extracted with EtOAc (3×10 mL). The combined organic layers were dried (MgSO₄), filtered, and concentrated. The residue was purified on silica gel (1:2 hexane-ethyl acetate) to afford (6-{[(4-chlorophthalazin-1-yl)oxy]methyl}pyridin-2-yl)methanol as a yellow solid: LRMS (ESI) m/z 303 (303 calcd for C₁₅H₁₂ClN₃O₂, M+H).

### Step 3: Synthesis of (6-{[(4-chlorophthalazin-1-yl)oxy]methyl}pyridin-2-yl)methyl methanesulfonate

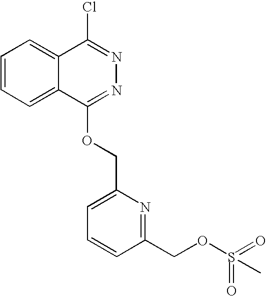

A solution of CH₂Cl₂ (5 mL) and (6-{[(4-chlorophthalazin-1-yl)oxy]methyl}pyridin-2-yl)methanol (480 mg, 1.60 mmol) was treated with triethylamine (0.12 ml, 1.75 mmol), followed by methanesulfonyl chloride (0.24 ml, 1.75 mmol). After 1 hr, the mixture was partitioned between saturated aqueous NaHCO₃ (10 mL) and CH₂Cl₂ (20 mL). The phases were separated and the aqueous layer was extracted with CH₂Cl₂ (3×10 mL). The combined organic layers were dried (MgSO₄), filtered, and concentrated to afford (6-{[(4-chlorophthalazin-1-yl)oxy]methyl}pyridin-2-yl)methyl methanesulfonate as a yellow solid: LRMS (ESI) m/z 380 (380 calcd for C₁₆H₁₄ClN₃O₄S, M+H).

### Step 4: Synthesis of N-[(6-{[(4-chlorophthalazin-1-yl)oxy]methyl}pyridin-2-yl)methyl]-2-phenylethanamine

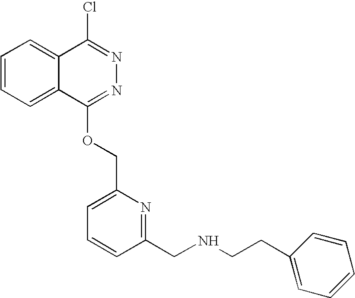

A solution of (6-{[(4-chlorophthalazin-1-yl)oxy]methyl}pyridin-2-yl)methyl methanesulfonate (200 mg, 0.53 mmol) and DMF (5 mL) was treated with phenethylamine (1 mL, 8.5 mmol) at rt. After 12 h, the mixture was partitioned between saturated aqueous NaHCO₃ (10 mL) and EtOAc (20 mL). The phases were separated and the aqueous layer was extracted with EtOAc (3×10 mL). The combined organic layers were dried (MgSO₄), filtered, and concentrated. The residue was purified on silica gel (9:1 dichloromethane-methanol) to yield yellow solid. The free base was dissolved in HCl (5 mL, 1 N in ether) and the solution was filtered to afford N-[(6-{[(4-chlorophthalazin-1-yl)oxy]methyl}pyridin-2-yl)methyl]-2-phenylethanamine as a yellow solid: ¹H NMR (500 MHz, CD₃OD) δ 8.42-8.40 (m, 1H), 8.37-8.36 (m, 1H), 8.15-8.10 (m, 2H), 7.95-7.86 (m, 2H), 7.70 (m, 1H), 7.43-7.13 (m, 1H), 7.36-7.20 (m, 4H), 5.8 (s, 2H), 4.45 (s, 2H), 3.39-3.60 (m, 2H), 3.07-3.06 (m, 2H); LRMS (ESI) m/z 405 (405 calcd for C₂₃H₂₁ClN₄O, M+H).

### Step 5: Synthesis of 2-phenyl-N-[(6-{[(4-pyridin-3-ylphthalazin-1-yl)oxy]methyl}pyridin-2-yl)methyl]ethanamine

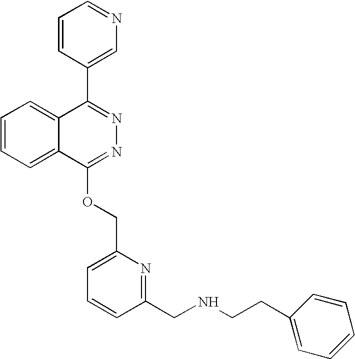

The solution of N-[(6-{[(4-chlorophthalazin-1-yl)oxy]methyl}pyridin-2-yl)methyl]-2-phenylethanamine (300 mg, 0.74 mmol) and DMF:H₂O (6 mL, 2:1 mixture) was degassed via argon for 10 min. Then K₂CO₃ (256 mg, 1.85 mmol), Pd(Ph₃P)₄ (86 mg, 0.074 mmol), and 2-pyridylboronic acid (136 mg, 1.11 mmol) were added to the solution at rt and the resulting mixture was then heated at 75° C. After 1 h, the reaction mixture was cooled to 22° C., then filtered through a pad of Celite. The filtrate was concentrated under reduced pressure and purified on silica gel (9:1 dichloromethane-methanol) to afford 2-phenyl-N-[(6-{[(4-pyridin-3-ylphthalazin-1-yl)oxy]methyl}pyridin-2-yl)methyl]ethanamine as a yellow oil: ¹NMR (500 MHz, CDCl₃) δ 8.91 (d, 1H), 8.71-8.70 (dd, 1H), 8.35-8.31 (d, 1H), 8.03-8.00 (m, 1H), 7.89-7.79 (m, 3H), 7.64-7.61 (m, 1H), 7.45-7.42 (m, 2H), 7.23-7.11 (m, 6H), 5.81 (s, 2H), 3.94 (s, 2H), 3.05-2.91 (m, 2H), 2.84-2.81 (m, 2H), 2.26 (br, 2H); LRMS (ESI) m/z 448 (448 calcd for C₂₈H₂₅N₅O, M+H).

### Example 41

### 2-Phenyl-N-[(6-{[(4-pyridin-4-ylphthalazin-1-yl)oxy]methyl}pyridin-2-yl)methyl]ethanamine

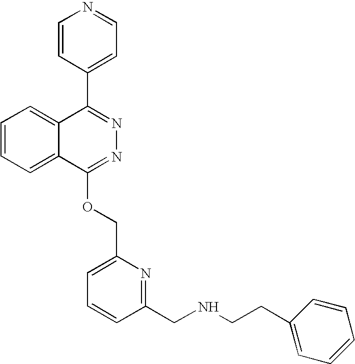

Utilizing the general procedure outlined in Step 5 of Example 40, N-[(6-{[(4-chlorophthalazin-1-yl)oxy]methyl}pyridin-2-yl)methyl]-2-phenylethanamine and 4-pyridylboronic acid were used to afford 2-phenyl-N-[(6-{[(4-pyridin-4-ylphthalazin-1-yl)oxy]methyl}pyridin-2-yl)methyl]ethanamine as a yellow oil: ¹H NMR (500 MHz, CDCl₃) δ 8.78-8.72 (m, 2H), 8.36-8.33 (d, 1H), 7.88-7.79 (m, 3H), 7.64-7.58 (m, 3H), 7.44-7.42 (d, 1H), 7.23-7.12 (m, 6H), 5.78 (s, 2H), 3.94 (s, 2H), 2.94-2.92 (m, 2H), 2.91-2.80 (m, 2H), 2.80 (br, 2H); LRMS (ESI) m/z 448 (448 calcd for C₂₈H₂₅N₅O, M+H).

### Example 42

### 2-Phenyl-N-[(6-{[(4-pyrimidin-5-ylphthalazin-1-yl)oxy]methyl}pyridin-2-yl)methyl]ethanamine

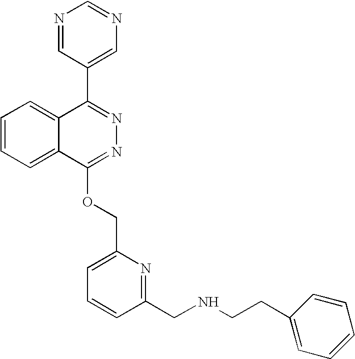

Utilizing the general procedure outlined in Step 5 of Example 40, N-[(6-{[(4-chlorophthalazin-1-yl)oxy]methyl}pyridin-2-yl)methyl]-2-phenylethanamine and pyrimidin-5-ylboronic acid were used to afford 2-phenyl-N-[(6-{[(4-pyrimidin-5-ylphthalazin-1-yl)oxy]methyl pyridin-2-yl)methyl]ethanamine as a yellow solid: ¹H NMR (500 MHz, CDCl₃) δ 9.32 (s, 1H), 9.08 (s, 2H), 8.39-8.33 (d, 1H), 7.93-7.82 (m, 3H), 7.65-7.62 (m, 1H), 7.44-7.42 (d, 1H), 7.23-7.12 (m, 6H), 5.82 (s, 2H), 3.96 (s, 2H), 3.01-2.93 (m, 2H), 2.85-2.81 (m, 2H), 2.40 (br, 2H); LRMS (ESI) m/z 449 (449 calcd for C₂₇H₂₄N₆O, M+H).

### Example 43

### N-{[6-({[4-(2,3-Dichlorophenyl)phthalazin-1-yl]oxy}methyl)pyridin-2-yl]methyl}-2-phenylethanamine

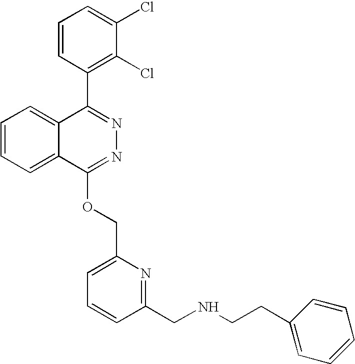

Utilizing the general procedure outlined in Step 5 of Example 40, N-[(6-{[(4-chlorophthalazin-1-yl)oxy]methyl}pyridin-2-yl)methyl]-2-phenylethanamine and 2,3-dichlorobenzeneboronic acid were used to afford N-{[6-({[(2,3dichlorophenyl)phthalazin-1-yl]oxy}methyl)pyridin-2-yl]methyl}-2-phenylethanamine as a yellow solid: ¹H NMR (500 MHz, CDCl₃) δ 8.41-8.40 (s, 1H), 7.91-7.88 (t, 1H), 7.87-7.82 (t, 1H), 7.73-7.02 (t, 1H), 7.68-7.66 (m, 1H), 7.55-7.52 (t, 2H), 7.45-7.39 (m, 2H), 7.32-7.22 (m, 6H), 5.89 (s, 2H), 4.00 (s, 2H), 3.01-2.97 (m, 2H), 2.91-2.88 (m 2H), 2.06 (br, 2H); LRMS (ESI) m/z 515 (515 calcd for C₂₉H₂₄Cl₂N₄O, M+H).

### Example 44

### 2-Phenyl-N-[(6-{[(4-pyridin-2-ylphthalazin-1-yl)oxy]methyl}pyridin-2-yl)methyl]ethanamine

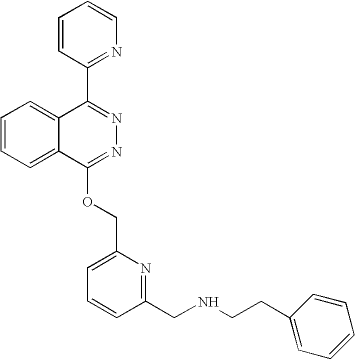

To a solution of degassed THF (10 mL) was added N-[(6-{[(4-chlorophthalazin-1-yl)oxy]methyl}pyridin-2-yl)methyl]-2-phenylethanamine (300 mg, 0.74 mmol), 2-pyridylzinc bromide (2.2 mL of 0.5M solution in THF, 3.3 mmol), and tetrakis(triphenylphosphine) paladium(0) (86 mg, 1.85 mmol). The mixture was further degassed with argon for an additional 30 minutes and heated at reflux under an argon atmosphere overnight. The reaction mixture was cooled to rt and concentrated in vacuo. The mixture was partitioned between saturated saturated aqueous NaHCO₃ (10 mL) and EtOAc (20 mL). The phases were separated and the aqueous layer was extracted with EtOAc (3×10 mL). The combined organic layers were dried (MgSO₄), filtered, and concentrated. The residue was purified on silica gel (9:1 dichloromethane-methanol) to afford 2-phenyl-N-[(6-{[(4-pyridin-2-ylphthalazin-1-yl)oxy]methyl}pyridin-2-yl)methyl]ethanamine as a colorless oil: ¹H NMR (500 MHz, CDCl₃) δ 8.91 (d, 1H), 8.66-8.60 (d, 1H), 8.25-8.23 (m, 1H), 8.25-8.23 (m, 1H), 7.82-7.75 (m, 3H), 7.60-7.57 (m, 1H), 7.38-7.37 (m, 1H), 7.32-7.31 (m, 1H), 7.19-7.10 (m, 6H), 5.66 (s, 2H), 4.0 (s, 2H), 2.99-2.93 (m, 2H), 2.90-2.86 (m, 2H); LRMS (ESI) m/z 448 (448 calcd for C₂₈H₂₅N₅O, M+H).

### Example 45

### N,N-Dimethyl-4-[(6-{[(2-phenylethyl)amino]methyl}pyridin-2-yl)methoxy]phthalazin-1-amine

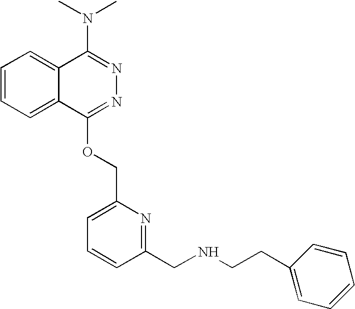

### Step 1: Synthesis of 4-{[6-({[tert-butyl(dimethyl)silyl]oxy}methyl)pyridin-2-yl]methoxy}-N,N-dimethylphthalazin-1-amine

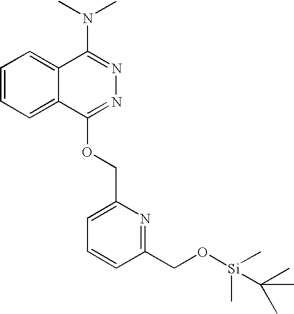

A mixture of 1-{[6-({[tert-butyl(dimethyl)silyl]oxy}methyl)pyridin-2-yl]methoxy}-4-chlorophthalazine (250 mg, 0.6 mmol), dimethylamine (3 ml, 2.0 M in THF), triethylamine (83 ul, 0.6 mmol) and catalytic amount of potassium iodide in n-butanol (5 mL) in sealed pressure tube was heated at 110° C. for overnight. Concentrated and purified on silica gel (1:2 hexane-ethyl acetate) to provide 4-{[6-({[tert-butyl(dimethyl)silyl]oxy}methyl)pyrridin-2-yl]methoxy)}-N,N-dimethylphthalazin-1-amine as a white solid: LRMS (ESI) m/z 425 (425 calcd for C₂₃H₃₂SiN₄O₂, M+H).

### Step 2: Synthesis of [6-({[4-(dimethylamino)phthalazin-1-yl]oxy}methyl)pyridin-2-yl]methanol

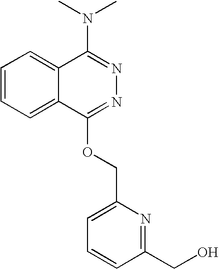

Utilizing the general procedure outlined in Step 2 of Example 40, 4-{[6-({[tert-butyl(dimethyl)silyl]oxy}methyl)pyridin-2-yl]methoxy}-N,N-dimethylphthalazin-1-amine and TBAF to give [6-({[4-(dimethylamino)phthalazin-1-yl]oxy}methyl)pyridin-2-yl]methanol as a colorless oil: LRMS (ESI) m/z 311 (311 calcd for C₁₇H₁₈N₄O₂, M+H).

### Step 3: Synthesis of N,N-dimethyl-4-[(6-{[(2-phenylethyl)amino]methyl}pyridin-2-yl)methoxy]phthalazin-1-amine

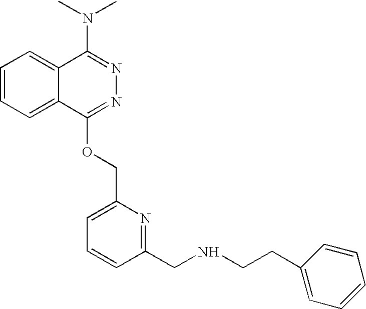

Utilizing the general procedure outlined in Steps 3 and 4 of Example 40, N,N-dimethyl-4-[(6-{[(2-phenylethyl)amino]methyl}pyridin-2-yl)methoxy]phthalazin-1-amine was synthesized to afford N,N-dimethyl-4-[(6-{[(2-phenylethyl)amino]methyl}pyridin-2-yl)methoxy]phthalazin-1-amine as a HCl salt: ¹H NMR (500 MHz, CD₃OD) δ 8.87-8.83 (m, 1H), 8.48-8.46 (m, 1H), 8.21-8.18(m, 1H), 78.15-8.11 (m, 1H), 8.01-7.98 (m, 1H), 7.76-7.74 (d, 1H), 7.51-7.50 (d, 1H), 7.35-7.32 (m, 2H), 7.29-7.25 (m, 3H), 5.68 (s, 2H), 4.48 (s, 2H), 3.51 (s, 6H), 3.39-3.36 (m, 2H), 3.08-3.05 (m, 2H); LRMS (ESI) m/z 414 (414 calcd for C₂₅H₂₇N₅O, M+H).

### Example 46

### 2-Phenyl-N-{[6-({[4-(1H-1,2,4-triazol-1-yl)phthalazin-1-yl]oxy}methyl)pyridin-2-yl]methyl}ethanamine

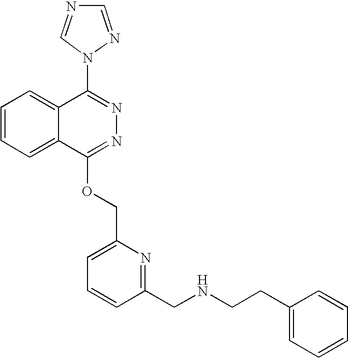

### Step 1: Synthesis of 1-chloro-4-(1H-1,2,4-triazol-1-yl)phthalazine:

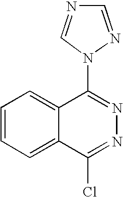

A mixture of 1,4-dichorophthalazine (1 g, 5.0 mmol) and 1,2,4-triazole (0.35 g, 5.0 mmol) in DMF at 0° C. was treated with sodium hydride (0. 13 g, 5.0 mmol). After 10 min, the reaction mixture was warmed to rt and allowed to stir for 2 h. The mixture was partitioned between saturated aqueous NaHCO₃ (20 mL) and EtOAc (60 mL). The phases were separated and the aqueous layer was extracted with EtOAc (3×60 mL). The combined organic layers were dried (MgSO₄), filtered, and concentrated. The residue was purified on silica gel (2:1 hexane-ethyl acetate) to provide 1-chloro-4-(1H-1,2,4-triazol-1-yl)phthalazine a white solid: LRMS (ESI) m/z 232 (232 calcd for C₁₀H₆ClN₅, M+H).

### Step 2: Synthesis of 1-{[6-({[tert-butyl(dimethyl)silyl]oxy}methyl)pyridin-2-yl]methoxy}-4-(1H-1,2,4-triazol-1-yl)phthalazine

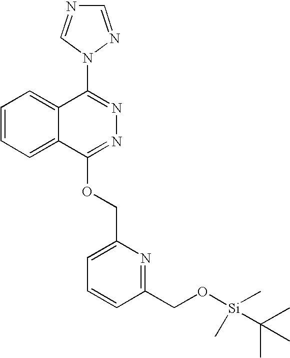

Utilizing the general procedure outlined in Step 1 of Example 40, 1-chloro-4-(1H-1,2,4-triazol-1-yl)phthalazine afforded 1-{[6-({[tert-butyl(dimethyl)silyl]oxy}methyl)pyridin-2-yl]methoxy}-4-(1H-1,2,4-triazol-1-yl)phthalazine as a colorless oil: LRMS (ESI) m/z 449 (449 calcd for C₂₃H₂₈N₆O₂Si, M+H).

### Step 3: Synthesis of [6-({[4-(1H-1,2,4-triazol-1-yl)phthalazin-1-yl]oxy}methyl)pyridin-2-yl]methanol

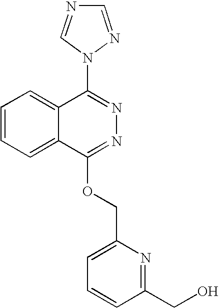

Utilizing the general procedure outlined in Step 2 of Example 40, 1-{[6-({[tert-butyl(dimethyl)silyl]oxy}methyl)pyridin-2-yl]methoxy}-4-(1H-1,2,4-triazol-1-yl)phthalazine afforded [6-({[4-(1H-1,2,4-triazol-1-yl)phthalazin-1-yl]oxy}methyl)pyridin-2-yl]methanol as a colorless oil: LRMS (ESI) m/z 335 (335 calcd for C₁₇H₁₄N₆O₂, M+H).

### Step 4: Synthesis of [6-({[4-(1H-1,2,4-triazol-1-yl)phthalazin-1-yl]oxy}methyl)pyridin-2-yl]methyl methanesulfonate

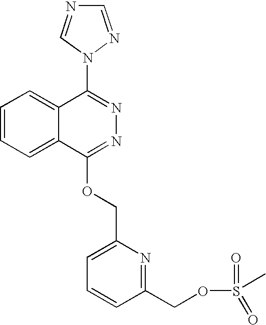

Utilizing the general procedure outlined in Step 3 of Example 40, [6-({[4-(1H-1,2,4-triazol-1-yl)phthalazin-1-yl]oxy}methyl)pyridin-2-yl]methanol afforded [6-({[4-(1H-1,2,4-triazol-1-yl)phthalazin-1-yl]oxy}methyl)pyridin-2-yl]methyl methanesulfonate as a colorless oil: LRMS (ESI) m/z 412 (412 calcd for C₁₈H₁₆N₆O₄S, M+H).

### Step 5: Synthesis of 2-phenyl-N-{[6-({[4-(1H-1,2,4-triazol-1-yl)phthalazin-1-yl]oxy}methyl)pyridin-2-yl]methyl}ethanamine

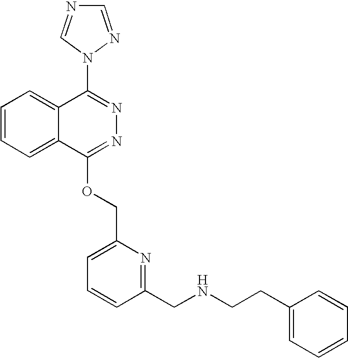

Utilizing the general procedure outlined in Step 4 of Example 40, [6-({[4-(1H-1,2,4-triazol-1-yl)phthalazin-1-yl]oxy}methyl)pyridin-2-yl]methyl methanesulfonate and phenethylamine afforded 2-phenyl-N-{[6-({[4-1H-1,2,4-triazol-1-yl)phthalazin-1-yl]oxy}methyl)pyridin-2-yl]methyl}ethanamine as a colorless oil: ¹H NMR (500 M}, CDCl₃) δ 9.09 (s, 1H), 8.79-8.77 (m, 1H), 8.42-8.40 (m, 1H), 8.27 (s, 1H), 7.99-7.95 (m, 2H), 7.71-7.68 (t, 1H), 7.50-7.49 (d, 1H), 7.30-7.27 (m, 3H), 7.22-7.20 (m, 3H), 5.85 (s, 2H), 3.98 (s, 2H), 2.97-2.95 (m, 2H), 2.88-2.85 (m 2H), 2.20 (br, 2H); LRMS (ESI) m/z 438 (438 calcd for C₂₅H₂₃N₇O, M+H).

### Example 47

### 2-Phenyl-N-{[6-({[4-(1H-pyrazol-1-yl)phthalazin-1-yl]oxy}methyl)pyridin-2-yl]methyl}ethanamine

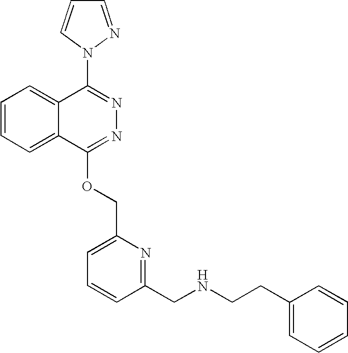

Utilizing the general procedure outlined in Example 46, 1,4-dichlorophthalazine and pyrazole gave 2-phenyl-N-{[6-({[4-(1H-pyrazol-1-yl)phthalazin-1-yl]oxy}methyl)pyridin-2-yl]methyl}ethanamine as a colorless oil: ¹H NMR (500 MHz, CDCl₃) δ 8.92-8.88 (m, 1H), 8.42 (m, 1H), 8.30-8.29 (m, 1H), 7.94-7.83 (m, 3H), 7.65-7.62 (t, 1H), 7.41-7.40 (d, 1H), 7.23-7.20 (m, 3H), 7.20-7.16 (m, 3H), 6.50-6.48 (m, 1H), 5.71 (s, 2H), 4.00 (s, 2H), 2.99-2.94 (m, 2H), 2.90-2.88 (m 2H); LRMS (ESI) m/z 437 (437 calcd for C₂₈H₂₄N₆O, M+H).

### Example 48

### 2-Phenyl-N-{[6-({[4-(1H-pyrrol-1-yl)phthalazin-1-yl]oxy}methyl)pyridin-2-yl]methyl}ethanamine

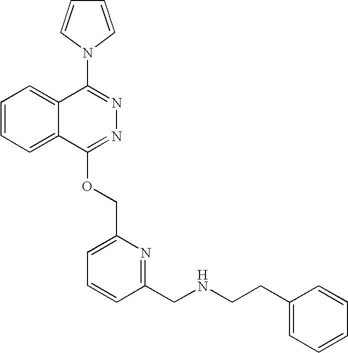

Utilizing the general procedure outlined in Example 46, 1,4-dichlorophthalazine and pyrrole gave 2-phenyl-N-{[6-({[4-(1H-pyrrol-1-yl)phthalazin-1-yl]oxy}methyl)pyridin-2-yl]methyl}ethanamine as a colorless oil: ¹H NMR (500 MHz, CDCl₃) δ 8.31-8.30 (m, 1H), 8.06-8.03 (m, 1H), 7.88-7.85 (m, 2H), 7.65-7.63 (t, 1H), 7.43-7.42 (d, 1H), 7.24-7.01 (m, 9H), 6.40-6.38 (m, 2H), 5.70 (s, 2H), 4.01 (s, 2H), 3.06-3.00 (m, 2H), 2.91-2.90 (m 2H); LRMS (ESI) m/z 436 (436 calcd for C₂₇H₂₅N₅O, M+H).

### Example 49

### N-{[6-({[4-(1H-Imidazol-1-yl)phthalazin-1-yl]oxy}methyl)pyridin-2-yl]methyl}-2-phenylethanamine

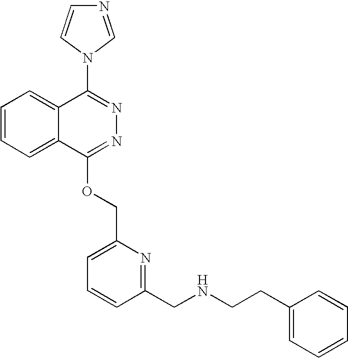

Utilizing the general procedure outlined in Example 46, 1,4-dichlorophthalazine and imidazole gave N-{[6-({[4-(1H-imidazol-1-yl)phthalazin-1-yl]oxy}methyl)pyridin-2-yl]methyl}-2-phenylethanamine as a colorless oil: ¹H NMR (500 MHz, CDCl₃) δ 8.36-8.31 (m, 1H), 8.00 (s, 1H), 7.93-7.85 (m, 3H), 7.65-7.62 (t, 1H), 7.44 (m, 1H), 7.41-7.40 (d, 1H), 7.26 (s, 1H), 7.24-7.20 (m, 3H), 7.16-7.12 (m, 3H), 5.75 (s, 2H), 3.96 (s, 2H), 2.98-2.92 (m, 2H), 2.85-2.83 (m 2H), 2.46 (br, 2H); LRMS (ESI) m/z 437 (437 calcd for C₂₆H₂₄N₆O, M+H).

### Example 50

### 2-Phenyl-N-{[6-({[4-(2H-1,2,3-triazol-2-yl)phthalazin-1-yl]oxy}methyl)pyridin-2-yl]methyl}ethanamine and 2-Phenyl-N-{[6-({[4-(1H-1,2,3-triazol-1-yl)phthalazin-1-yl]oxy}methyl)pyridin-2-yl]methyl}ethanamine

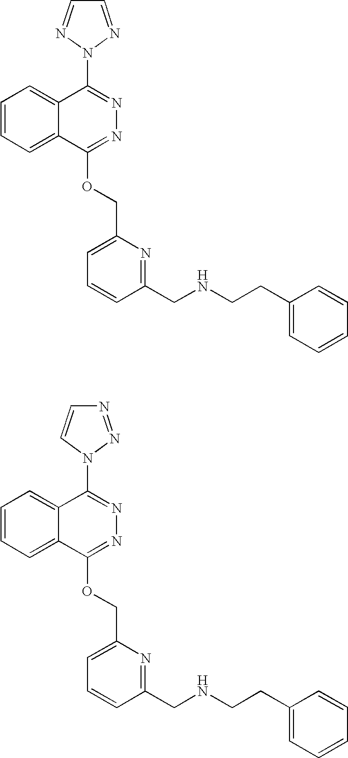

Utilizing the general procedure outlined in Example 46, 1,4-dichlorophthalazine and 1,2,3-1H-triazole afforded 2-phenyl-N-{[6-({[4-(2H-1,2,3-triazol-2-yl)phthalazin-1-yl]oxy}methyl)pyridin-2-yl]methyl}ethanamine and 2-phenyl-N-{[6-({[4-(1H-1,2,3-triazol-1-yl)phthalazin-1-yl]oxy}methyl)pyridin-2-yl]methyl}ethanamine as a inseparable mixture of isomers by silica gel chromatography: ¹H NMR (500 MHz, CDCl₃, 2:1 mixture of isomers) δ 8.78-8.77 (m, 0.3H), 8.51-8.50 (d, 0.3H), 8.34-8.32 (m, 0.6H), 8.27-8.25 (m, 0.6H), 7.80 (s, 1H), 7.93-7.85 (m, 4H), 7.63-7.60 (m, 1H), 7.43-7.38 (m, 1H), 7.22-7.11 (m, 6H), 5.78 (m, 2H), 3.93 (m, 2H), 2.97-2.91 (m, 2H), 2.85-2.80 (m, 2H), 2.54 (br, 2H); LRMS (ESI) m/z 438 (438 calcd for C₂₅H₂₃N₇O, M+H).

### Example 51

### 4-[(6-{[(2-Phenylethyl)amino]methyl}pyridin-2-yl)methoxy]phthalazin-1-ol

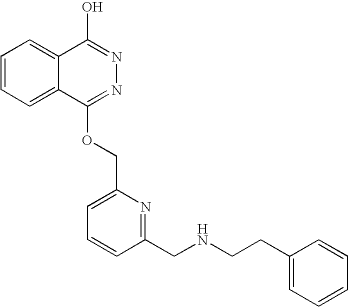

### Step 1: Synthesis of (6-{[(4-methoxyphthalazin-1-yl)oxy]methyl}pyridin-2-yl)methanol

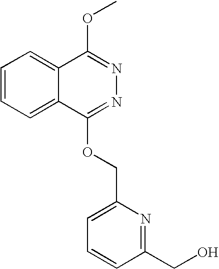

A mixture of 1-{[6-({[tert-butyl(dimethyl)silyl]oxy}methyl)pyridin-2-yl]methoxy}-4-chlorophthalazine (250 mg, 0.6 mmol) and sodium methoxide (1.8 mL, 0.5 M in methanol) in methanol (2 mL) was heated in a sealed pressure tube to 100° C. After 12 h, the reaction mixture was concentrated and the mixture was partitioned between saturated aqueous NaHCO₃ (10 mL) and EtOAc (20 mL). The phases were separated and the aqueous layer was extracted with EtOAc (3×10 mL). The combined organic layers were dried (MgSO₄), filtered, and concentrated to afford (6-{[(4-methoxyphthalazin-1-yl)oxy]methyl}pyridin-2-yl)methanol as a colorless oil: LRMS (ESI) m/z 298 (298 calcd for C₁₆H₁₅N₃O₃, M+H).

### Step 2: Synthesis of (6-{[(4-methoxyphthalazin-1-yl)oxy]methyl}pyridin-2-yl)methyl methanesulfonate

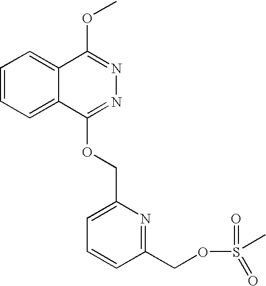

Utilizing the general procedure outlined in Step 4 of Example 46, (6-{[(4-methoxyphthalazin-1-yl)oxy]methyl}pyridin-2-yl)methanol afforded (6-{[(4-methoxyphthalazin-1-yl)oxy]methyl}pyridin-2-yl)methyl methanesulfonate as a yellow solid: LRMS (ESI) m/z 376 (376 calcd for C₁₇H₁₇N₃O₅S, M+H).

### Step 3: Synthesis of N-[(6-{[(4-methoxyphthalazin-1-yl)oxy]methyl}pyridin-2-yl)methyl]-2-phenylethanamine

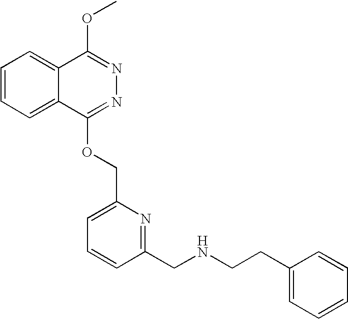

Utilizing the general procedure outlined in Step 5 of Example 46, (6-{[(4-methoxyphthalazin-1-yl)oxy]methyl}pyridin-2-yl)methyl methanesulfonate provided N-[(6-{[(4-methoxyphthalazin-1-yl)oxy]methyl}pyridin-2-yl)methyl]-2-phenylethanamine as a colorless oil: ¹H NMR (500 MHz, CDCl₃) δ 8.23-8.21 (m, 1H), 8.16-8.15 (m, 1H), 7.86-7.84 (m, 2H), 7.70-7.67 (t, 1H), 7.45-7.44 (d, 1H), 7.31-7.07 (m, 4H), 5.70 (s, 2H), 4.21 (s, 3H), 4.03 (s, 2H), 3.03-3.01 (m, 2H), 2.95-2.92 (m, 2H), 2.83 (br, 2H); LRMS (IS) m/z 401 (401 calcd for C₂₄H₂₄N₄O₂, M+H).

### Step 4: Synthesis of 4-[(6-{[(2-phenylethyl)amino]methyl}pyridin-2-yl)methoxy]phthalazin-1-ol

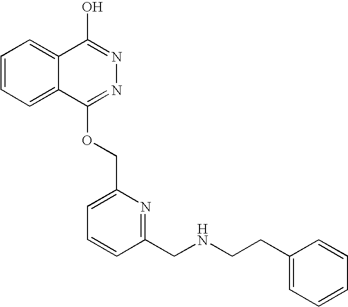

A solution of N-[(6-{[(4-methoxyphthalazin-1-yl)oxy]methyl}pyridin-2-yl)methyl]-2-phenylethanamine (20 mg, mmol) and CH₂Cl₂ (0.5 ml) was treated with HCl (1 mL, 1 N in Et₂O). The resulting precipitate was collected and dried in vacuo to afford 4-[(6-{[(2-phenylethyl)amino]methyl}pyridin-2-yl)methoxy]phthalazin-1-ol as a yellow solid: ¹H NMR (500 Hz, CDCl₃) δ 10.12 (br, 1H), 8.42-8.41 (m, 1H), 8.10-8.09 (m, 1H), 7.84-7.81 (m, 2H), 7.70-7.67 (t, 1H), 7.42-7.41 (d, 1H), 7.31-7.26 (m, 3H), 7.23-7.20 (m, 3H), 5.51 (s, 2H), 4.21 (s, 3H), 4.03 (s, 2H), 3.07-3.00 (m, 2H), 2.93-2.90 (m, 2H), 2.33 (br, 2H); LRMS (ESI) m/z 387 (387 calcd for C₂₃H₂₂N₄O₂, M+H).

### Example 52

### 2-Phenyl-N-({6-[([1,2,4]triazolo[3,4-a]phthalazin-6-yloxy)methyl]pyridin-2-yl}methyl)ethanamine

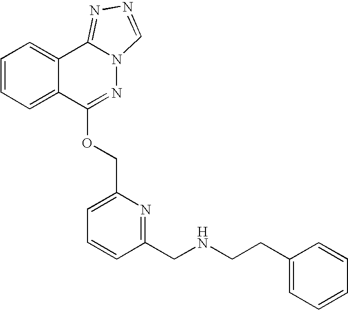

### Step 1: Synthesis of 6-chloro[1,2,4]triazolo[3,4-a]phthalazine

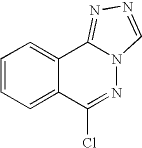

A solution of 1-chloro-4-hydrazinophthalazine (0.5 g, 3.2 mmol) (PCT Int. Appl. WO 02/42305 A1, 2001) and triethyl orthoformate (10 mL) was refluxed for 4 hr. The solution was concentrated and resulting solid was washed with ethanol to afford 6-chloro[1,2,4]triazolo[3,4-a]phthalazine as a yellow solid: LRMS (ESI) m/z 205 (205 calcd for C₉H₅N₄Cl, M+H).

### Step 2: Synthesis of 6-{[6-({[tert-butyl(dimethyl)silyl]oxy}methyl)pyridin-2-yl]methoxy}[1,2,4]triazolo[3,4-a]phthalazine

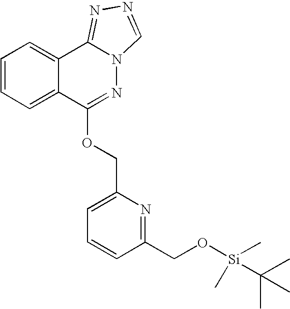

Utilizing the general procedure outlined in Step 2 of Example 46, 6-chloro[1,2,4]triazolo[3,4-a]phthalazine provided 6-{[6-({[tert-butyl(dimethyl)silyl]oxy}methyl)pyridin-2-yl]methoxy}[1,2,4]triazolo[3,4-a]phthalazine as a white solid: LRMS (ESI) m/z 421 (421 calcd for C₂₂H₂₇N₅O₂Si, M+H).

### Step 3: Synthesis of {6-[([1,2,4]triazolo[3,4-a]phthalazin-6-yloxy)methyl]pyridin-2-yl}methanol

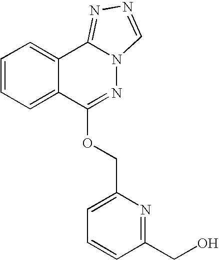

Utilizing the general procedure outlined in Step 3 of Example 46, 6-{[6-{[tert-butyl(dimethyl)silyl]oxy}methyl)pyridin-2-yl]methoxy}[1,2,4]triazolo[3,4-a]phthalazine afforded {6-[([1,2,4]triazolo[3,4-a]phthalazin-6-yloxy)methyl]pyridin-2-yl}methanol as a white solid: LRMS (ESI) m/z 308(308 calcd for C₁₆H₁₃N₅O₂, M+H).

### Step 4: Synthesis of {6-[([1,2,4]triazolo[3,4-a]phthalazin-6-yloxy)methyl]pyridin-2-yl}methyl methanesulfonate

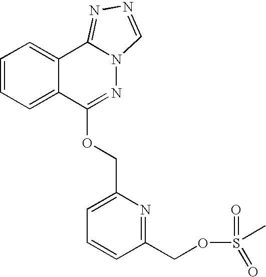

Utilizing the general procedure outlined in Step 4 of Example 46, {6-[([1,2,4]triazolo[3,4-a]phthalazin-6-yloxy)methyl]pyridin-2-yl}methanol provided {6-[([1,2,4]triazolo[3,4-a]phthalazin-6-yloxy)methyl]pyridin-2-yl}methyl methanesulfonate as a white solid: LRMS (ESI) m/z 386(386 calcd for C₁₇H₁₅N₅O₄S, M+H).

### Step 5: Synthesis of 2-phenyl-N-({6-[([1,2,4]triazolo[3,4-a]phthalazin-6-yloxy)methyl]pyridin-2-yl}methyl)ethanamine

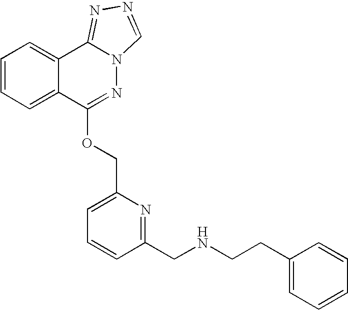

Utilizing the general procedure outlined in Step 5 of Example 46, {6-[([1,2,4]triazolo[3,4-a]phthalazin-6-yloxy)methyl]pyridin-2-yl}methyl methanesulfonate afforded 2-phenyl-N-({6-[([1,2,4]triazolo[3,4-a]phthalazin-6-yloxy)methyl]pyridin-2-yl}methyl)ethanamine as a HCl salt: ¹H NMR (500 MHz, CD₃OD) δ 9.96 (br, 1H), 8.61-8.60 (d, 1H), 8.54-8.52 (d, 1H), 8.23 (t, 1H), 8.15 (t, 1H), 8.02-7.99 (t, 1H), 7.80-7.78 (d, 1H), 7.51-7.49 (d, 1H), 7.35-7.32 (m, 2H), 7.29-7.27 (m, 3H), 5.81 (s, 2H), 4.85 (s, 2H), 3.40-3.67 (m, 2H), 3.30-3.04 (m, 2H); LRMS (ESI) m/z 411 (411 calcd for C₂₄H₂₂N₆O, M+H).

### Example 53

### 2-Phenyl-N-{[6-{[2-trifluoromethyl)imidazo[2,1-a]phthalazin-6-yl]oxy}methyl)pyridin-2-yl]methyl}ethanamine

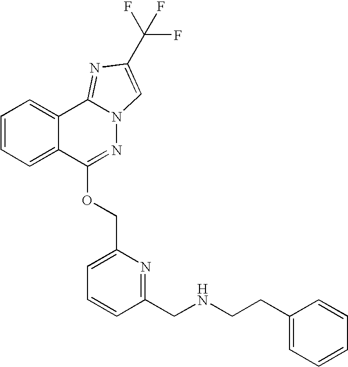

### Step 1: Synthesis of 6-chloro-2-(trifluoromethyl)imidazo[2,1-a]phthalazine

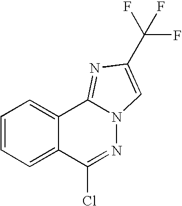

The mixture of 4-chlorophthalazin-1-amine (0.5 g, 2.8 mmol) (Tetrahedron Lett. 1996, 37, 4065) and 3-bromo-1,1,1-trifluoroacetone (0.5 g, 2.8 mmol) in ethanol (10 mL) was heated to reflux. After 12 h, the mixture was partitioned between saturated aqueous NaHCO₃ (10 mL) and EtOAc (20 mL). The phases were separated and the aqueous layer was extracted with EtOAc (3×20 mL). The combined organic layers were dried (MgSO₄), filtered, and concentrated. The residue was purified on silica gel (5:1 hexane-ethyl acetate) to afford 6-chloro-2-(trifluoromethyl)imidazo[2,1-a]phthalazine as a white solid: LRMS (ES) m/z 272 (272 calcd for C₁₁H₅ClN₃O₃, M+H).

### Step 2: Synthesis of 2-phenyl-N-{[6-({[2-(trifluoromethyl)imidazo[2,1-a]phthalazin-6-yl]oxy}methyl)pyridin-2-yl]methyl}ethanamine

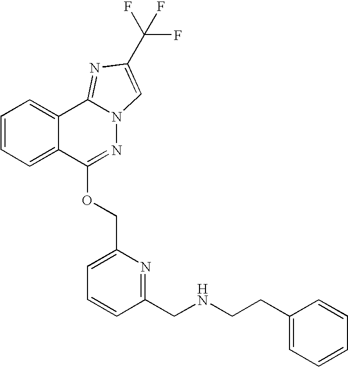

Utilizing the general procedure outlined in Example 46, 6-chloro-2-(trifluoromethyl)imidazo[2,1-a]phthalazine gave 2-phenyl-N-{[6-({[2-(trifluoromethyl)imidazo[2,1-a]phthalazin-yl]oxy}methyl)pyridin-2-yl]methyl}ethanamine as a white solid: ¹H NMR (500 MHz, CDCl₃) δ 8.57-8.55 (d, 1H), 8.27-8.25 (d, 1H), 7.97 (s, 1H), 7.93-7.88 (m, 1H), 7.74-7.71 (m, 2H), 7.42-7.41 (d, 1H), 7.29-7.26 (m, 3H), 7.22-7.13 (m, 3H), 5.66 (s, 2H), 3.93 (s, 2H), 2.98-2.96 (m, 2H), 2.88-2.84 (m, 2H), 1.76 (br, 2H); LRMS (ES1) m/z 478 (478 calcd for C₂₆H₂₂F₃N₅O, M+H).

### Example 54

### N-(3-{[(3-Methylimidazo[5,1-a]phthalazin-6-yl)oxy]methyl}benzyl)-2-phenylethanamine

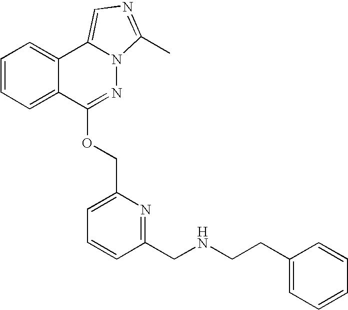

### Step 1: Synthesis of (4E)-4-(3-oxo-2-benzofuran-1(3H)-ylidene)-2-phenyl-1,3-oxazol-5(4H)-one

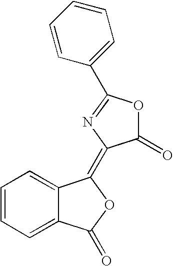

A mixture of phthalic anhydride (10 g, 67.5 mmol), Hippuric acid (12 g, 67.5 mmol) and sodium acetate (5.5 g, 67.5 mmol) in acetic anhydride (50 ml) was vigorously stirred at 100° C. After 2 h, the mixture was filtered while hot, washed the solid with hot water, and washed with acetone until filtrate became colorless. The yellow-orange solid was further washed with ether and dried in vacuo to afford (4E)-4-(3-oxo-2-benzofuran-1(3H)-ylidene)-2-phenyl-1,3-oxazol-5(4H)-one as a yellow-orange solid: LRMS (ESI) m/z 292 (292 calcd for C₁₇H₉O₄N, M+H).

### Step 2: Synthesis of N-[2-hydrazino-2-oxo-1-(4-oxo-3,4-dihydrophthalazin-1-yl)ethyl]benzamide

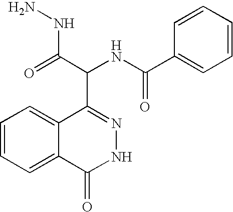

(4E)-4-(3-oxo-2-benzofuran-1(3H)-ylidene)-2-phenyl-1,3-oxazol-5(4H)-one (5.3 g, 8.9 mmol) was added in portions to a stirred hydrazine hydrate (14 mL) over 10 min at 0° C. After 10 min, the cold bath was removed and the mixture was heated to 110° C. After 5 min, the reaction mixture was cooled to rt and the resulting solid was filtered. The solid was washed with ethanol followed by ether and then dried to afford N-[2-hydrazino-2-oxo-1-4-oxo-3,4-dihydrophthalazin-1-yl)ethyl]benzamide as a white solid: LRMS (ESI) m/z 338 (338 calcd for C₁₇H₁₅N₅O₃, M+H).

### Step 3: Synthesis of 4-aminomethyl)phthalazin-1(2H)-one

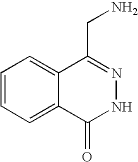

N-[2-hydrazino-2-oxo-1-(4-oxo-3,4-dihydrophthalazin-1-yl)ethyl]benzamide (3.0 g, 17 mmol) was treated with concentrated HCl (36 mL, 12 N) and heated to 105° C. After 12 h, the reaction mixture was cooled and basified with solid NaOH until pH 10. The aqueous solution was extracted with CH₂Cl₂ (4×50 mL) to afford 4-(aminomethyl)phthalazin-1(2H)-one as a yellow solid: LRMS (ESI) m/z 176 (176 calcd for C₉H₉N₃O, M+H).

### Step 4: Synthesis of 6-chloro-3-methyl-imidazo[5,1-a]phthalazine

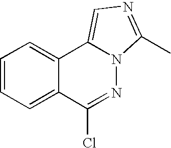

A solution of DMF (7 mL) and 4-(aminomethyl)phthalazin-1(2H)-one (0.6 g, 3.4 mmol) was treated with triethylamine (0.5 mL, 3.4 mmol) and acetyl chloride (0.24 mL, 3.4 mmol). After 2 h, the solid was filtered off and the filtrate was concentrated under vacuum The resulting brown solid N-[(4-oxo-3,4-dihydrophthalazin-1-yl)methyl]acetamide was treated with POCl₃ (5 mL) and heated to reflux. After 12 h, the reaction mixture was concentrated and partitioned between saturated aqueous NaHCO₃ (10 mL) and CH₂Cl₂ (30 mL). The phases were separated and the aqueous layer was extracted with CH₂Cl₂ (3×30 mL). The combined organic layers were dried (MgSO₄), filtered, and concentrated. The residue was purified on silica gel (1:2 hexane-ethyl acetate) to provide 6-chloro-3-methyl-imidazo[5,1-a]phthalazine as a brown solid: LRMS (ESI) m/z 218 (218 calcd for C₁₁H₈ClN₃, M+H).

### Step 5: Synthesis of N-(3-{[(3-methylimidazo[5,1-a]phthalazin-6-yl)oxy]methyl}benzyl)-2-phenylethanamine

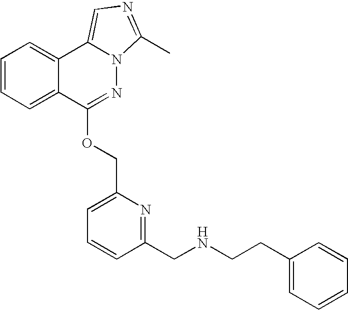

Utilizing the general procedure outlined in Example 46, 6-chloro-3-methyl-imidazo[5,1-a]phthalazine gave N-(3-{[(3-methylimidazo[5,1-a]phthalazin-6-yl)oxy]methyl}benzyl)-2-phenylethanamine as a white solid: ¹H NMR (500 MHz, CDCl₃) δ. 8.11-8.10 (d, 1H), 8.88-8.87 (d, 1H), 7.73-7.68 (m, 2H), 7.58 (s, 1H), 7.48-7.44 (m, 2H), 7.30-7.27 (m, 3H), 7.21-7.20 (m, 3H), 5.62 (s, 2H), 4.02 (s, 2H), 3.02-2.96 (m, 2H), 2.91-2.89 (m, 2H), 2.62 (s, 3H).

### Example 55

### N-({6-[(Imidazo[2,1-a]phthalazin-6-yloxy)methyl]pyridin-2-yl}methyl)-2-phenylethanamine

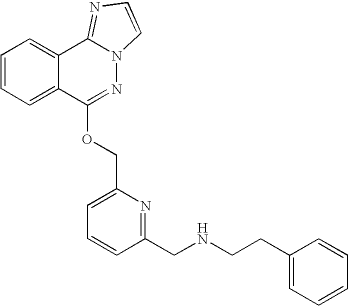

### Step 1: Synthesis of 6-chloroimidazo[2,1-a]phthalazine

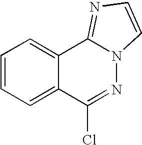

The mixture of 4-chlorophthalazin-1-amine (0.5 g, 2.8 mmol) (Tetrahedron Lett. 1996, 37, 4065), sodium bromide (86 mg, 0.8 mmol) and chloroacetaldehyde (0.4 mL, 2.8 mmol, 50% in H₂O) in ethanol (5 mL) was heated to 70° C. After 12 h, the reaction mixture was concentrated and purified on silica gel (1:2 ethyl acetate-hexane) to afford 6-chloroimidazo[2,1-a]phthalazine as a yellow solid: LRMS (ESI) m/z 204 (204 calcd for C₁₀H₆ClN₃, M+H).

### Step 2: Synthesis of N-({6-[(imidazo[2,1-a]phthalazin-6-yloxy)methyl]pyridin-2-yl}methyl)-2-phenylethanamine

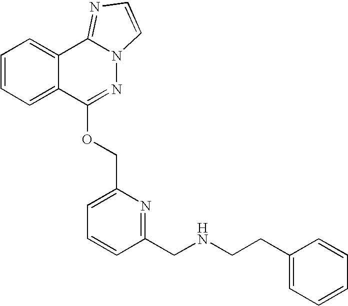

Utilizing the general procedure outlined in Example 46, 6-chloroimidazo[2,1-a]phthalazine provided N-({6-[(imidazo[2,1-a]phthalazin-6-yloxy)methyl]pyridin-2-yl}methyl)-2-phenylethanamine as a colorless oil: ¹H NMR (500 MHz, CDCl₃) δ 8.49-8.48 (d, 1H), 8.21-8.20 (d, 1H), 7.85-7.82 (t, 1H), 7.57-7.67 (m, 2H), 7.65-7.62 (t, 1H), 7.50 (s, 1H), 7.43-7.42 (d, 1H), 7.30-7.20 (m, 6H), 5.61 (s, 2H), 3.98 (s, 2H), 2.98-2.95 (m, 2H), 2.89-2.83 (m, 2H), 2.84 (br, 2H); LRMS (ESI) m/z 411 (411 calcd for C₂₅H₂₃N₅O, M+H).

### Example 56

### 2-Phenyl-N-{[6-({[3-(trifluoromethyl)[1,2,4]triazolo[3,4-a]isoquinolin-6-yl]oxy}methyl)pyridin-2-yl]methyl}ethanamine

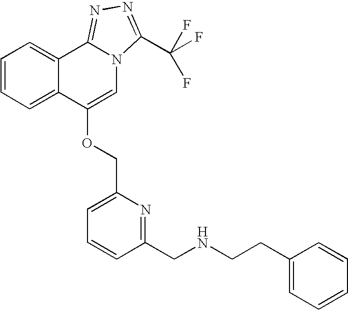

### Step 1: Synthesis of 4-{[6-({[tert-butyl(dimethyl)silyl]oxy}methyl)pyridin-2-yl]methoxy}-1-chloroisoquinoline

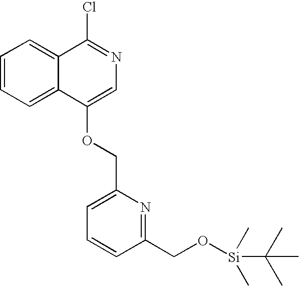

The mixture of 1-chloroisoquinolin-4-ol (0.5 g, 2.8 mmol) and [6-({[tert-butyl(dimethyl)silyl]oxy}methyl)pyridin-2-yl]methyl methanesulfonate (0.9 g, 2.8 mmol) in anhydrous DMF (15 mL) was treated with potassium hydroxide (0.16 g, 2.8 mmol) at rt. After 12, the mixture was partitioned between saturated aqueous NaHCO₃ (10 mL) and EtOAc (20 mL). The phases were separated and the aqueous layer was extracted with EtOAc (3×20 mL). The combined organic layers were dried (MgSO₄), filtered, and concentrated. The residue was purified on silica gel (20:1 hexane-ethyl acetate) to provide 4-{[6-({[tert-butyl(dimethyl)silyl]oxy}methyl)pyridin-2-yl]methoxy}-1-chloroisoquinoline as an orange oil: LRMS (ESI) m/z 416 (416 calcd for C₂₂H₂₇ClN₂O₂Si, M+H).

### Step 2: Synthesis of [6-({[3-(trifluoromethyl)[1,2,4]triazolo[3,4-a]isoquinolin-6-yl]oxy}methyl)pyridin-2-yl]methanol

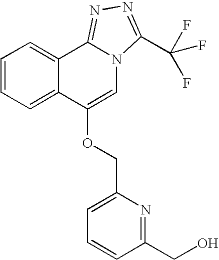

A solution of 4-{[6-({[tert-butyl(dimethyl)silyl]oxy}methyl)pyridin-2-yl]methoxy}-1-chloroisoquinoline (170 mg, 0.41 mmol), 2,2,2-trifluoroacetohydrazide (0.64 ml, 0.45 mmol) and HCl (5 mL, of 4 N in dioxane) in 1-butanol (3 mL) was heated to 110° C. After 5 h, the reaction mixture was concentrated and partitioned between saturated aqueous NaHCO₃ (10 mL) and EtOAc (20 mL). The phases were separated and the aqueous layer was extracted with EtOAc (3×20 mL). The combined organic layers were dried (MgSO₄), filtered and concentrated. The residue was purified on silica gel (1:2 hexane-ethyl acetate) to afford [6-{[3-(trifluoromethyl)[1,2,4]triazolo[3,4-a]isoquinolin-6-yl]oxy}methyl)pyridin-2-yl]methanol as a yellow solid: LRMS (ESI) m/z 375 (375 calcd for C₁₈H₁₃F₃N₄O₂, M+H).

### Step 3: Synthesis of [6-({[3-(trifluoromethyl)[1,2,4]triazolo[3,4-a]isoquinolin-6-yl]oxy}methyl)pyridin-2-yl]methyl methanesulfonate

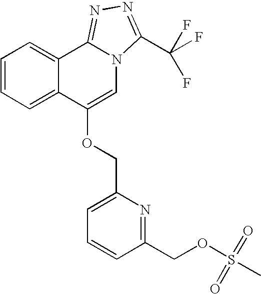

Utilizing the general procedure outlined in Example 46, [6-({[3-(trifluoromethyl)[1,2,4]triazolo[3,4-a]isoquinolin-6-yl]oxy}methyl)pyridin-2-yl]methanol afforded [6-({[3-(trifluoromethyl)[1,2,4]triazolo[3,4-a]isoquinolin-6-yl]oxy}methyl)pyridin-2-yl]methyl methanesulfonate as a yellow oil: LRMS (ESI) m/z 453 (453 calcd for C₁₉H₁₅F₃N₄O₄S, M+H).

### Step 4: Synthesis of 2-phenyl-N-{[6-{[3-trifluoromethyl)[1,2,4]triazolo(3,4-a]isoquinolin-6-yl]oxy}methyl)pyridin-2-yl]methyl}ethanamine

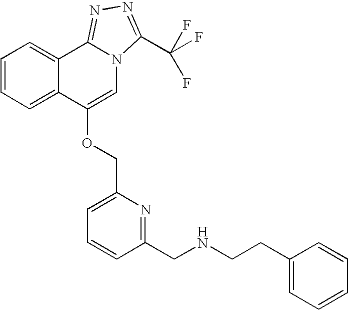

Utilizing the general procedure outlined in Example 46, [6-({[3-(trifluoromethyl)[1,2,4]triazolo[3,4-a]isoquinolin-6-yl]oxy}methyl)pyridin-2-yl]methyl methanesulfonate gave 2-phenyl-N-{[6-({[3-(trifluoromethyl)[1,2,4]triazolo[3,4-a]isoquinolin-6-yl]oxy}methyl)pyridin-2-yl]methyl}ethanamine as a yellow oil: ¹H NMR (500 MHz, CDCl₃) δ 8.80-8.78 (m, 1H), 8.29-8.26 (m, 1H), 7.83-7.81 (m, 2H), 7.76-7.25 (m, 1H), 7.64 (s, 1H), 7.46-7.45 (d, 1H), 7.31-7.21 (m, 6H), 5.31 (s, 2H), 3.93 (s, 2H), 2.998-2.96 (m, 2H), 2.88-2.86 (m, 2H), 1.88 (br, 2H); LRMS (ESI) m/z 478 (478 calcd for C₂₆H₂₂F₃N₅O, M+H).

### Example 57

### N-(3-{[(3-Methylimidazo[2,1-a]phthalazin-6-yl)oxy]methyl}benzyl)-2-phenylethanamine

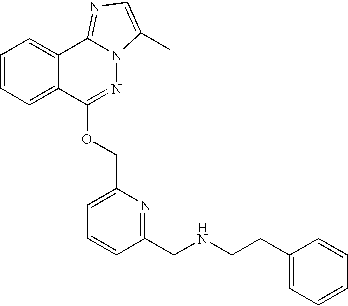

### Step 1: Synthesis of 6-chloro-3-methylimidazo[2,1-a]phthalazine

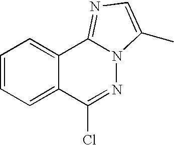

A mixture of 4-chlorophthalazin-1-amine (1.0 g, 5.6 mmol) (Tetrahedron Lett. 1996, 37, 4065), 2-chloropropionaldehyde (1.5 ml, 11.2 mmol), NaHCO₃ (2.1 g, 2.5 mmol) and HBr (0.5 mL, 48% in H₂O) in 1,4-dioxane (15 mL) was stirred vigorously at reflux. After 2 h, the reaction was cooled to rt and the mixture was partitioned between saturated aqueous NaHCO₃ (10 mL) and EtOAc (20 mL). The phases were separated and the aqueous layer was extracted with EtOAc (3×20 mL). The combined organic layers were dried (MgSO₄), filtered, and concentrated to afford 6-chloro-3-methylimidazo[2,1-a]phthalazine as an orange solid: LRMS (ESI) m/z 218 (218 calcd for C₁₁H₈ClN₃, M+H).

### Step 2: Synthesis of N-(3-{[(3-methylimidazo[2,1-a]phthalazin-6-yl)oxy]methyl}benzyl)-2-phenylethanamine

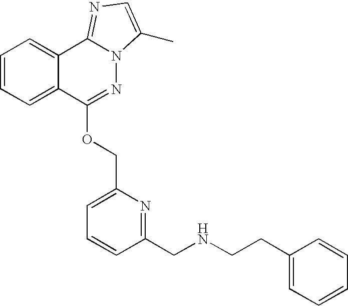

Utilizing the general procedure outlined in Example 46, 6-chloro-3-methylimidazo[2,1-a]phthalazine gave N-(3-{[(3-methylimidazo[2,1-a]phthalazin-6-yl)oxy]methyl}benzyl)-2-phenylethanamine as a white solid: ¹H NMR (500 MHz, CDCl₃) δ 8.45-8.44 (d, 1H), 8.17-8.16 (d, 1H), 7.81-7.78 (t, 1H), 7.71-7.69 (t, 1H), 7.49-7.47 (d, 2H), 7.30-7.25 (m, 4H), 7.21-7.18 (m, 3H), 5.66 (s, 2H), 3.97 (s, 2H), 2.96-2.94 (m, 2H), 2.93-2.87 (m, 2H), 2.47 (s, 3M, 2.11 (br, 2H); LRMS (ESI) m/z 424 (424 calcd for C₂₆H₂₅N₅O, M+H).

### Example 58

### Phenethyl-[6-(3-trifluoromethyl-[1,2,4]triazolo[3,4,-a]phthalazin-6-yloxymethyl)-pyridin-2-ylmethyl]-amine

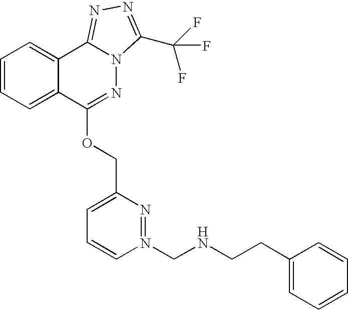

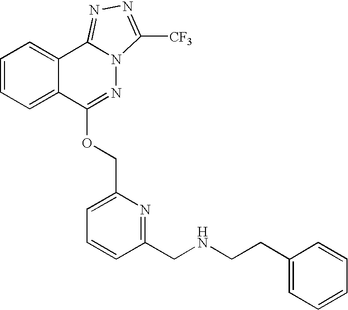

Utilizing the general procedure outlined in Example 5, 6-(3-trifluoromethyl-[1,2,4]triazolo[3,4-a]phthalazin-6-yloxymethyl)-pyridine-2-carbaldehyde (0.20 g, 0.54 mmol), phenethylamine (0.14 g, 1.07 mmol), sodium trisacetoxyborohydride (0.18 g, 0.86 mmol) and dichloroethane (4 mL) was reacted to give phenethyl-[6-(3-trifluoromethyl-[1,2,4]triazolo[3,4,-a]phthalazin-6-yloxymethyl)-pyridin-2-ylmethyl]-amine as a white solid: ¹H NMR (500 MHZ, CD₃OD) δ 8.65-8.64 (d, 1H), 8.44-8.43 (d, 1H), 8.16-8.12 (t, 1H), 8.04-7.93 (m, 2H), 7.79-7.77 (d, 1H), 7.47-7.46 (d, 1H), 7.34-7.31 (m, 2H), 7.27-7.24 (m, 3H), 5.74 (s, 2H), 4.47 (s, 2H), 3.38-3.35 (m 2H), 3.06-3.03 (m, 2H). LRMS (ESI) m/z 479 (479 calcd for C₂₅H₂₁N₆O, M+H).

Other variations or modifications, which will be obvious to those skilled in the art, are within the scope and teachings of this invention. This invention is not to be limited except as set forth in the following claims.

What is claimed is:

1. A compound of Formula (I) or Formula (II) 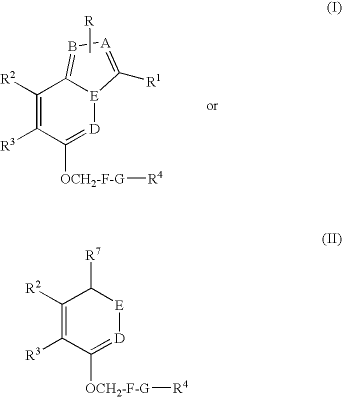
or a pharmaceutically acceptable salt thereof, wherein
A and B are each independently selected from the group consisting of CH₂, N and O;
D and E are each independently selected from the group consisting of N and O;
F is selected from the group consisting of phenyl and heteroaryl (pyridyl)
G a bond or is methylene, wherein the methylene optionally substituted with a substituent selected from methyl, ethyl, isopropyl, and carbonyl;
R is selected from the group consisting of
(a) H,
(b) CF₃,
(c) CH₃;
R¹ is selected from the group consisting of
(a) hydrogen,
(b) CF₃,
(c)phenyl,
(d) —C₁₋₆alkyl,
(e) —C₃₋₆cycloalkyl,
(f) —C₂₋₆alkenyl,
(g) —C₂₋₆alkynyl,
(h) —C₁₋₆alkyl,
(i) —C₂₋₆alkenyl,
(j) —C₁₋₆alkyl, and
(k) a heteroaromatic ring of 5 or 6 members, wherein the heteroaromatic ring comprises 1, 2 or 3 heteroatoms independently selected from the group consisting of N, and O, wherein the heteroaryl is optionally substituted with methyl, methoxy, hydroxyl or halo;
R² is selected from the group consisting of
(a) hydrogen,
(b) —C₁₋₆alkyl,
(c) heteroaromatic ring of 5 or 6 members, wherein the heterocycloalkyl or heteroaromatic ring comprises 1, 2 or 3 heteroatoms independently selected from the group consisting of N, and O,
(d) aryl, and
(e) —NR⁵R⁶;
R³ is selected from the group consisting of
(a) hydrogen,
(b) —C₁₋₆alkyl,
(c) heteroaromatic ring of 5 or 6 members, wherein the heterocycloalkyl or heteroaromatic ring comprises 1, 2 or 3 heteroatoms independently selected from the group consisting of N, and O,
(d) aryl, and
(e) —NR⁵R⁶,
wherein R² and R³ choices (a), (b), (c), (d) and (e) are each optionally substituted with one or two substituents selected from methyl, methoxy, halo and hydroxyl,
or R² and R³ are joined so that together with the atoms to which they are attached there is formed a ring selected from phenyl and cyclohexyl;
R⁴ is —NH(C₁₋₃alkylaryl), optionally substituted with one or two substituents selected from the group consisting of halo, hydroxyl, C₁₋₆alkyl and C₁₋₆alkyl;
R⁷ is selected from the group consisting of
(a) hydroxyl,
(b) N(CH₃)₂,
(c) Aryl,
d) a heteroaromatic ring of 5 or 6 members, wherein the heteroaromatic ring comprises 1, 2 or 3 heteroatoms independently selected from the group consisting of N and O, wherein R⁷ choice (b), (c) and (d) is optionally substituted with methyl, methoxy, hydroxyl or halo.

2. A compound according to claim 1 of Formula (1) 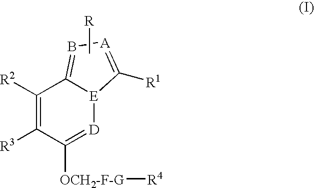
or a pharmaceutically acceptable salt thereof.

3. A compound according to claim 2 wherein:
D and E are N.

4. A compound according to claim 2 wherein:
G is methylene, wherein the methylene optionally substituted with a substituent selected from methyl, ethyl, isopropyl, and carbonyl.

5. A compound according to claim 2 wherein:
R¹ is selected from the group consisting of
(a) hydrogen,
(b) CF₃,
(c) phenyl,
(d) —C₁₋₃alkyl,
(e) C₃₋₆cycloalkyl.

6. A compound according to claim 2 wherein:
R² and R³ are joined so that together with the atoms to which they are attached there is formed a ring selected from phenyl and cyclohexyl.

7. A compound according to claim 2 wherein:
R⁴ is —NH(C₁₋₃alkylphenyl), optionally substituted with one or two substituents selected from the group consisting of halo, hydroxyl, —C₁₋₆allyl and —O—C₁₋₆alkyl.

8. A compound according to claim 2 wherein at least one of R² and R³ is phenyl.

9. A compound according to claim 2 wherein:
A and B are each independently selected from the group consisting of CH₂ and N;
D and E are each independently selected from the group consisting of N;
F is selected from the group consisting of phenyl and pyridyl;
G a bond or is methylene, wherein the methylene optionally substituted with a substituent selected from
methyl, ethyl, isopropyl, and carbonyl;
R is selected from the group consisting of
(a) H,
(b) CF₃,
(c) CH₃;
R¹ is selected from the group consisting of
(a) hydrogen,
(b) CF₃,
(c) phenyl,
(d) —C₁₋₃alkyl;
R² and R³ are joined so that together with the atoms to which they are attached there is formed a ring selected from phenyl and cyclohexyl;
R⁴ is —NH(C₁₋₃alkylphenyl), optionally substituted with one or two substituents selected from the group consisting of halo, hydroxyl, —C₁₋₆alkyl and —O—C₁₋₆alkyl.

10. A compound according to claim 1 wherein:
Within this aspect there is also a genus of compounds of Formula (II): 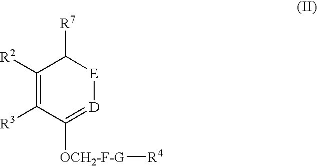
or a pharmaceutically acceptable salts thereof.

11. A compound according to claim 10 wherein:
D and E are N.

12. A compound according to claim 10 wherein:
G is methylene, wherein the methylene optionally substituted with a substituent selected from methyl, ethyl, isopropyl, and carbonyl.

13. A compound according to claim 10 wherein:
R⁴ is —NH(C₁₋₃alkylphenyl), optionally substituted with one or two substituents selected from the group consisting of halo, hydroxyl, C₁₋₆alkyl and —O—C₁₋₆alkyl.

14. A compound according to claim 10 wherein:
R⁷ is pyrrole, pyridine, or imidazole.aoptionally substituted with one or two substituents selected from methyl, methoxy, hydroxyl and halo.

15. A compound according to claim 10 wherein:
D and E are N;
G is methylene, wherein the methylene optionally substituted with a substituent selected from methyl, ethyl, isopropyl, and carbonyl;
R⁴ is —NH(C₁₋₃alkylphenyl), optionally substituted with one or two substituents selected from the group consisting of halo, hydroxyl, —C₁₋₆alkyl and —O—C₁₋₆alkyl;
R⁷ is pyrrole, pyridine, or imidazole.aoptionally substituted with one or two substituents selected from methyl, methoxy, hydroxyl and halo.

16. A compound according to claim 1 selected from the group consisting of: 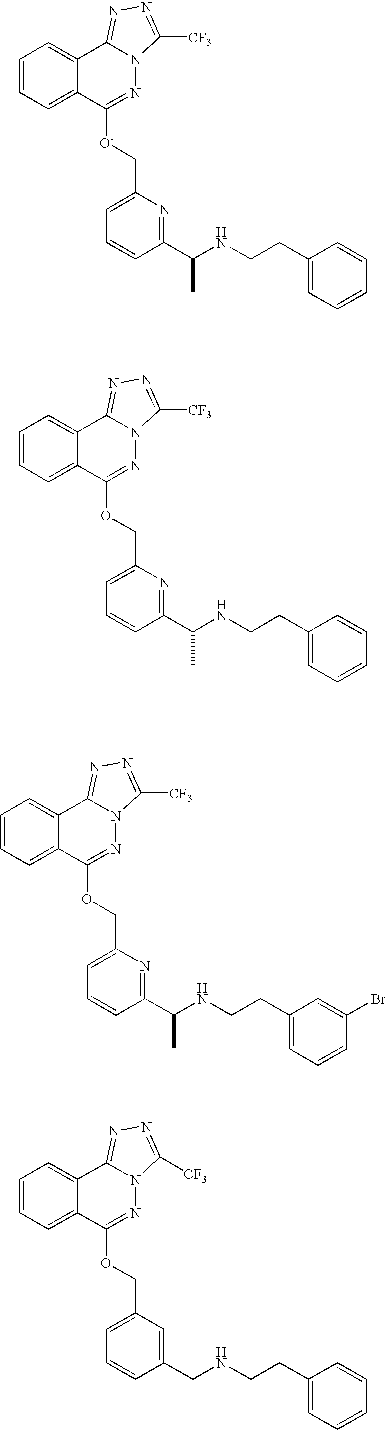 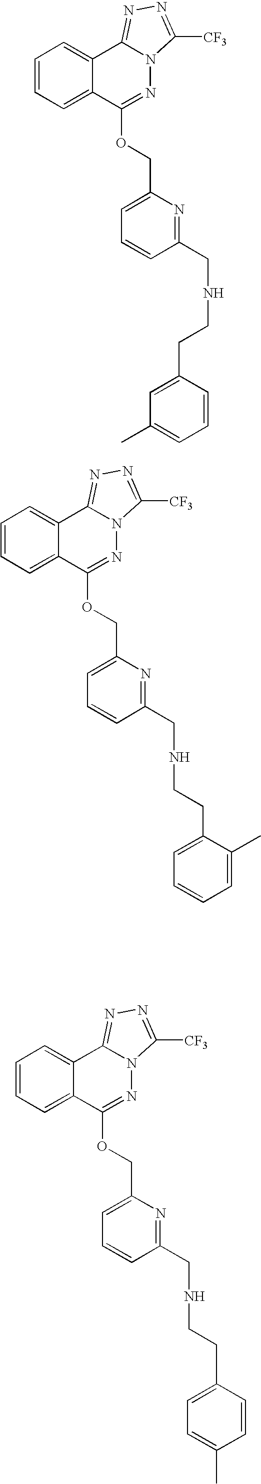 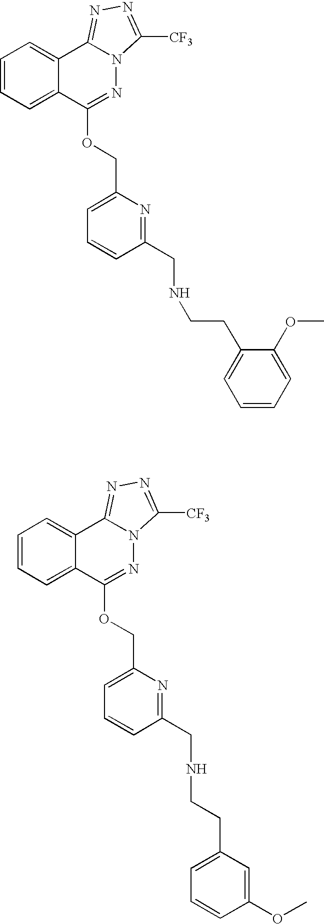 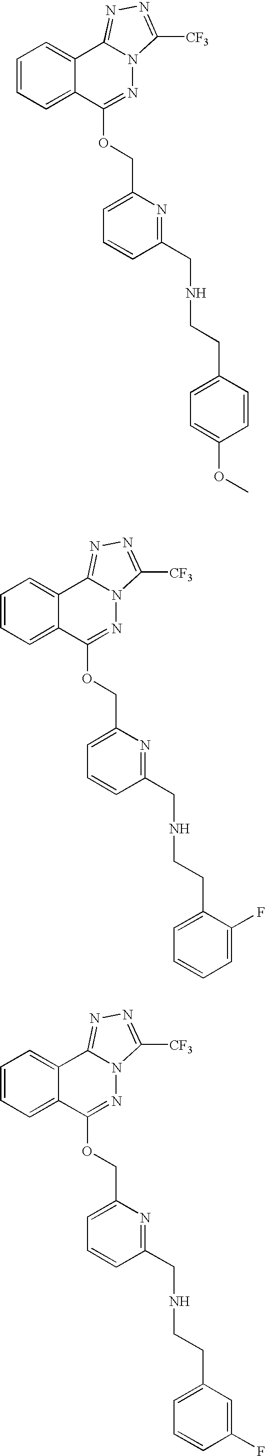 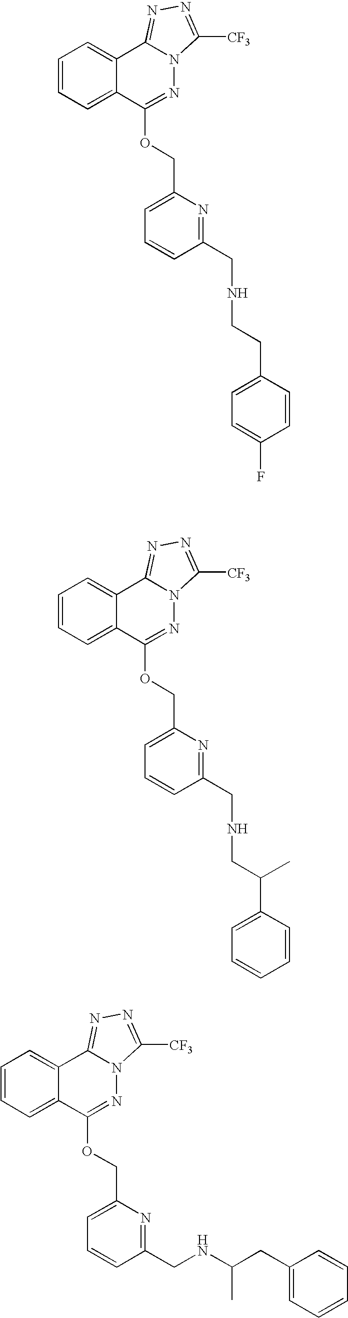 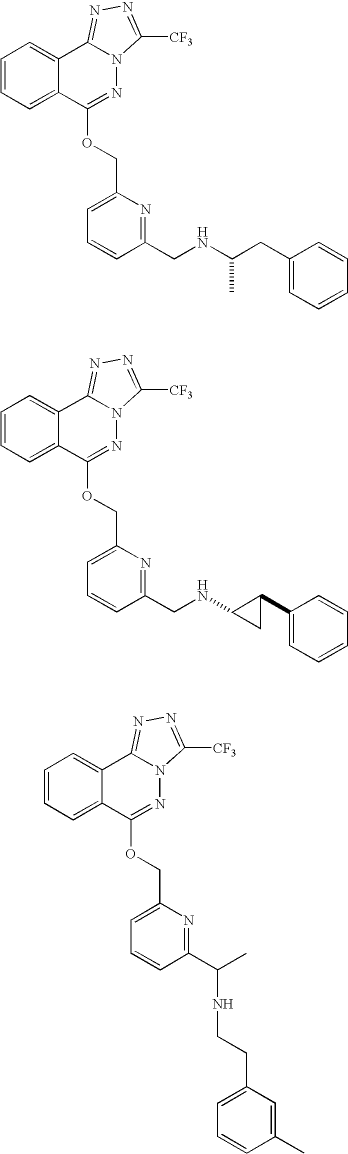 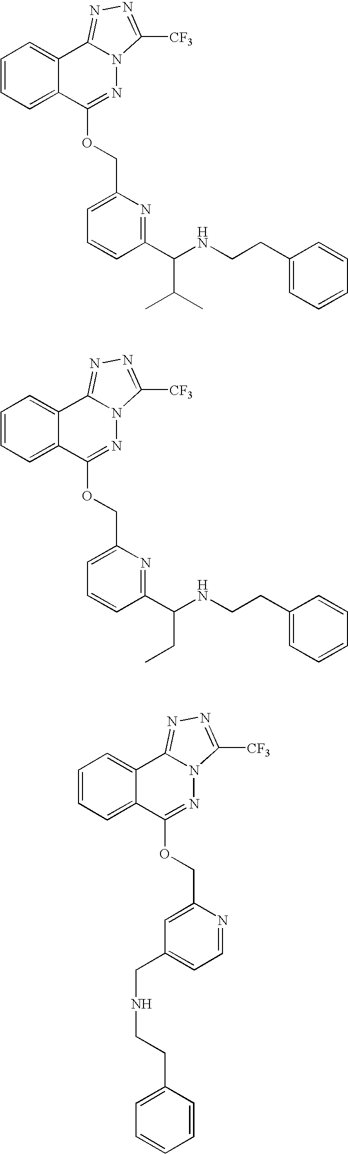 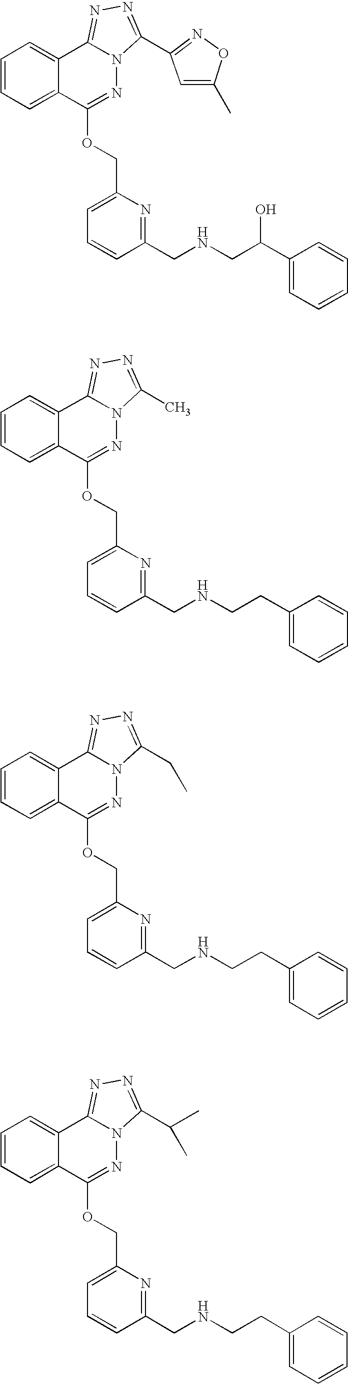 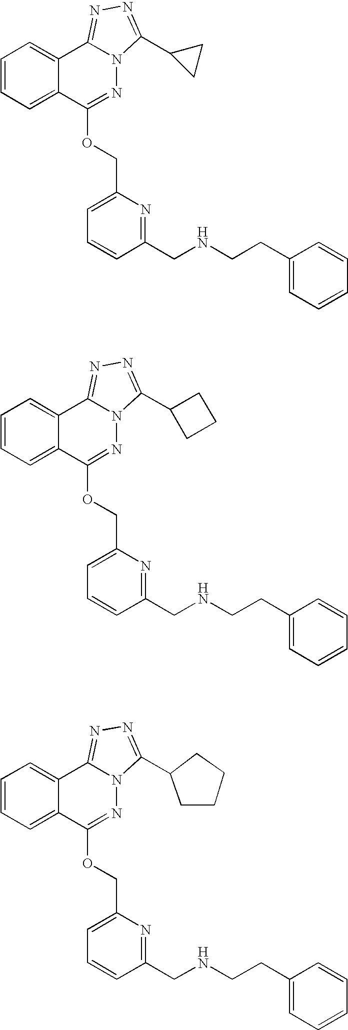 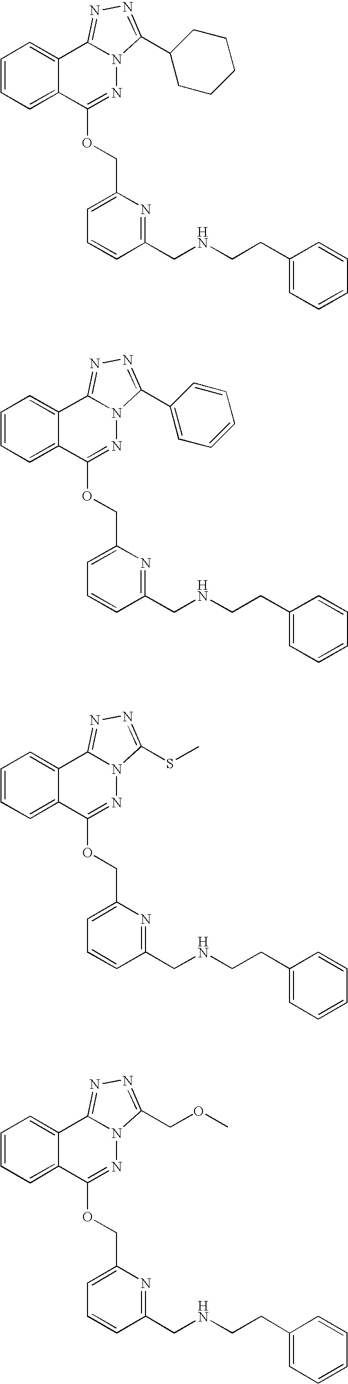 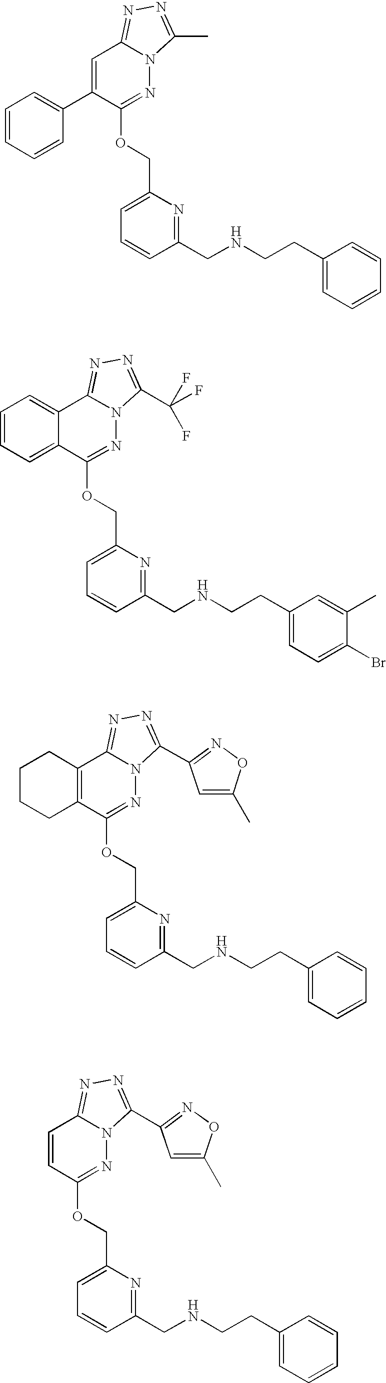 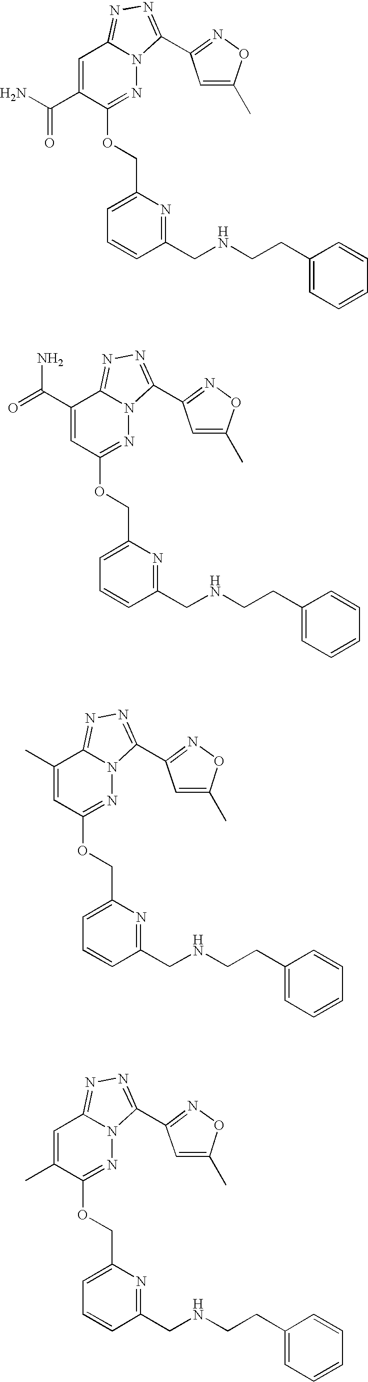 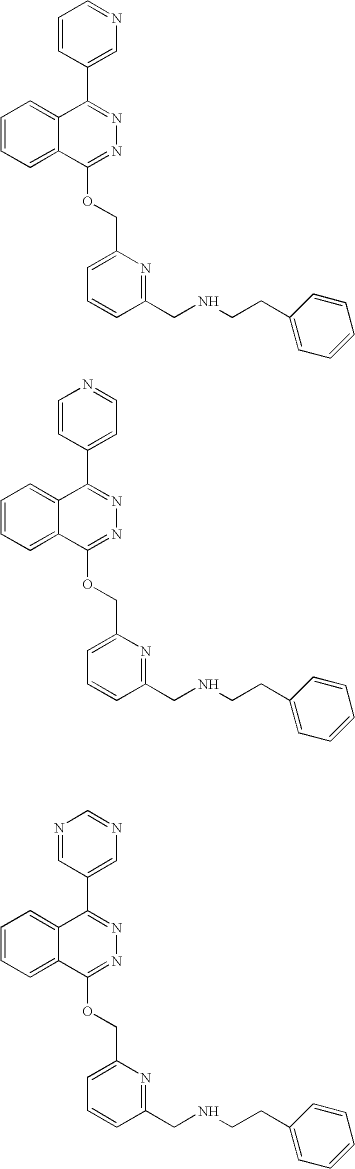 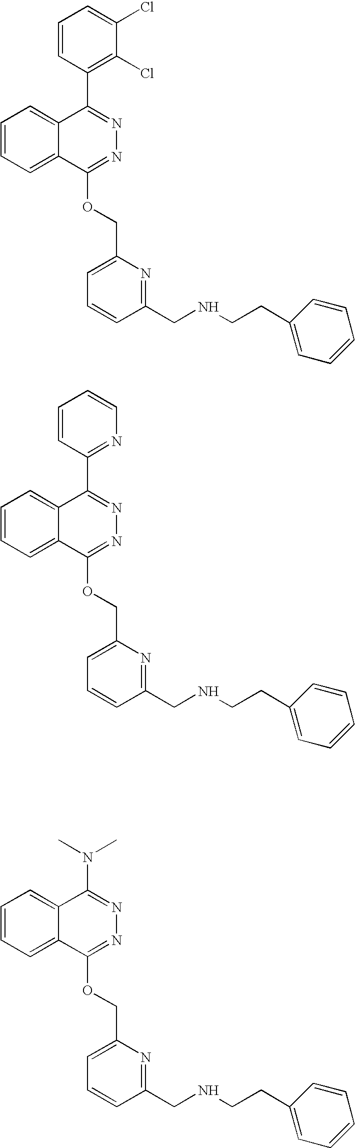 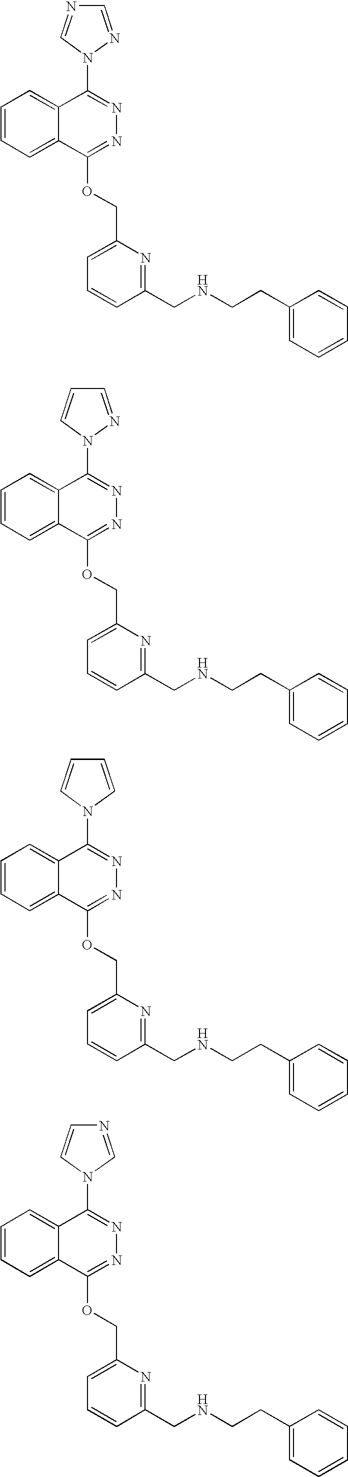 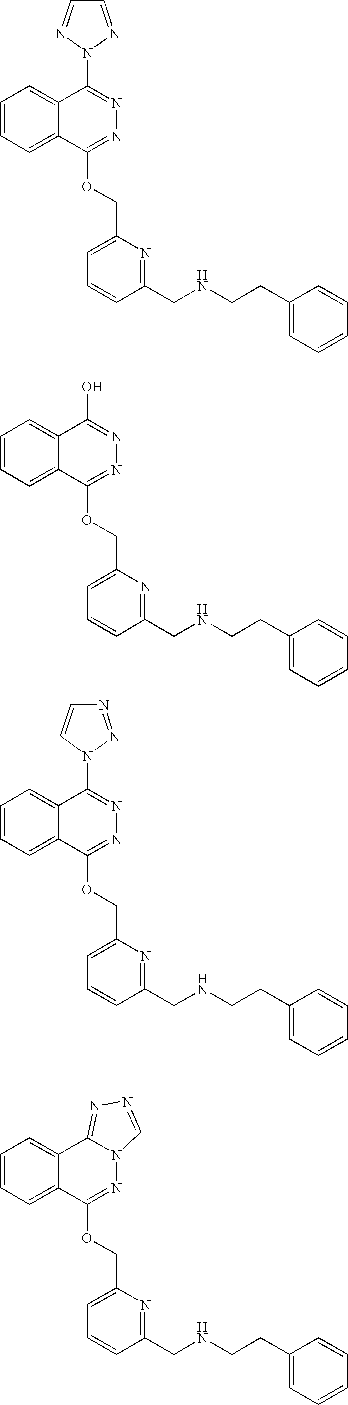 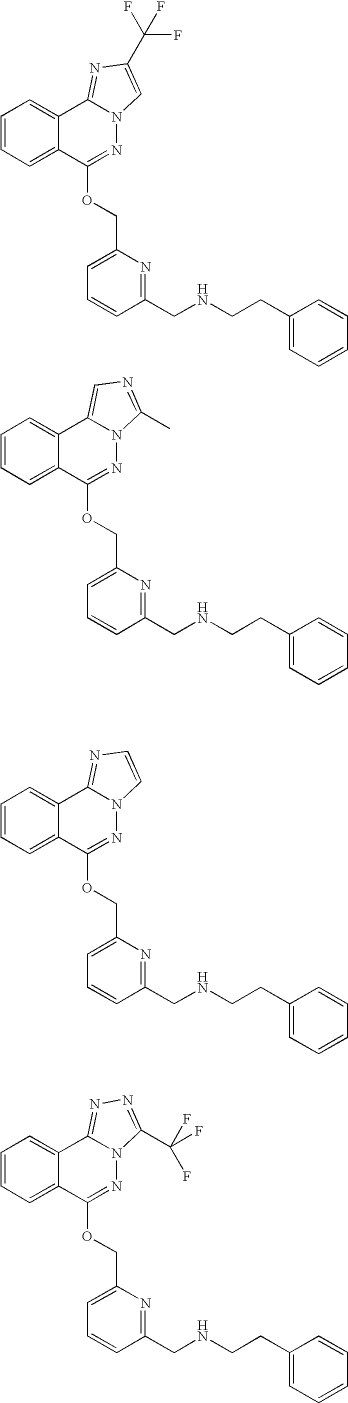 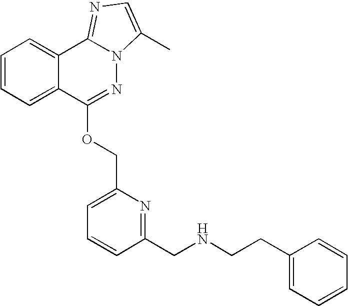
or a pharmaceutically acceptable salt thereof.

17. A pharmaceutical composition for treating an indication mediated by the binding of an α₂δ subunit of voltage gated calcium channel, comprising a therapeutically effective amount a of a compound according to claim 1 or a pharmaceutically acceptable salt thereof; and a pharmaceutically acceptable acrrier.

18. A composition according to claim 16, said composition further comprising i) an opiate agonist, ii) an opiate antagonist, iii) an mGluR5 antagonist, iv) a 5HT receptor agonist, v) a 5HT receptor antagonist, vi) a sodium channel antagonist, vii) an NMDA receptor agonist, viii) an NMDA receptor antagonist, ix) a COX-2 selective inhibitor, x) an NK1 antagonist, xi) a non-steroidal anti-inflammatory drug, xii) a GABA-A receptor modulator, xiii) a dopamine agonist, xiv) a dopamine antagonist, xv) a selective serotonin reuptake inhibitor, xvi) a tricyclic antidepressant drug, xvii) a norepinephrine modulator, xviii) L-DOPA, xix) buspirone, xx) a lithium salt, xxi) valproate, xxii) neurontin, xxiii) olanzapine, xxiv) a nicotinic agonist, xxv) a nicotinic antagonist, xxvi) a muscarinic agonist, xxvii) a muscarinic antagonist, xxviii) a selective serotonin and norepinephrine reuptake inhibitor (SSNRI), xxix) a heroin substituting drug, xxx) disulfiram, or xxxi) acamprosate.

19. A composition according to claim 17, wherein said heroin substituting drug is methadone, levo-alpha-acetylmethadol, buprenorphine or naltrexone.

20. A method of treatment of neuropathic pain comprising a step of administering an effective amount of a compound according to claim 1.

21. A method of treatment or prevention of pain comprising the step of administering a therapeutically effective amount, or a prophylactically effective amount, of the compound according to claim 1 or a pharmaceutically acceptable salt thereof.

22. A method of treatment or prevention of a pain disorder wherein said pain disorder is acute pain, persistent pain, chronic pain, inflammatory pain, or neuropathic pain, comprising the step of administering a therapeutically effective amount, or a prophylactically effective amount, of the compound according to claim 1 or a pharmaceutically acceptable salt thereof.

23. A method of treatment or prevention of anxiety, depression, bipolar disorder, psychosis, drug withdrawal, tobacco withdrawal, memory loss, cognitive impairment, dementia, Alzheimer's disease, schizophrenia or panic comprising the step of administering a therapeutically effective amount, or a prophylactically effective amount, of the compound according to claim 1 or a pharmaceutically acceptable salt thereof.

24. A method of treatment or prevention of disorders of extrapyramidal motor function comprising the step of administering a therapeutically effective amount, or a prophylactically effective amount, of the compound according to claim 1 or a pharmaceutically acceptable salt thereof.

25. The method of claim 24 wherein said disorder of extrapyramidal motor function is Parkinson's disease, progressive supramuscular palsy, Huntington's disease, Gilles de la Tourette syndrome, or tardive dyskinesia.

26. A method of treatment or prevention of anxiety disorders comprising the step of administering a therapeutically effective amount, or a prophylactically effective amount, of the compound according to claim 1 or a pharmaceutically acceptable salt thereof.

27. A method of claim 26 wherein said anxiety disorder is panic attack, agoraphobia or specific phobias, obsessive-compulsive disorders, post-traumatic stress disorder, acute stress disorder, generalized anxiety disorder, eating disorder, substance-induced anxiety disorder, or nonspecified anxiety disorder.

28. A method of treatment or prevention of neuropathic pain comprising the step of administering a therapeutically effective amount, or a prophylactically effective amount, of the compound according to claim 1 or a pharmaceutically acceptable salt thereof.

29. A method of treatment or prevention of Parkinson's Disease comprising the step of administering a therapeutically effective amount, or a prophylactically effective amount, of the compound according to claim 1 or a pharmaceutically acceptable salt thereof.

30. A method of treatment or prevention of depression comprising the step of administering a therapeutically effective amount, or a prophylactically effective amount, of the compound according to claim 1 or a pharmaceutically acceptable salt thereof.

31. A method of treatment or prevention of epilepsy comprising the step of administering a therapeutically effective amount, or a prophylactically effective amount, of the compound according to claim 1 or a pharmaceutically acceptable salt thereof.

32. A method of treatment or prevention of inflammatory pain comprising the step of administering a therapeutically effective amount, or a prophylactically effective amount, of the compound according to claim 1 or a pharmaceutically acceptable salt thereof.

33. A method of treatment or prevention of cognitive dysfunction comprising the step of administering a therapeutically effective amount, or a prophylactically effective amount, of the compound according to claim 1 or a pharmaceutically acceptable salt thereof.

34. A method of treatment or prevention of drug addiction, drug abuse and drug withdrawal comprising the step of administering a therapeutically effective amount, or a prophylactically effective amount, of the compound according to claim 1 or a pharmaceutically acceptable salt thereof.

35. A method of treatment or prevention of bipolar disorders comprising the step of administering a therapeutically effective amount, or a prophylactically effective amount, of the compound according to claim 1 or a pharmaceutically acceptable salt thereof.

36. A method of treatment or prevention of circadian rhythm and sleep disorders comprising the step of administering a therapeutically effective amount, or a prophylactically effective amount, of the compound according to claim 1 or a pharmaceutically acceptable salt thereof.

37. The method of claim 36 wherein the circadian rhythm and sleep disorders are shift-work induced sleep disorder or jet-lag.
